(19) United States
(12) Patent Application Publication
(10) Pub. No.: US 2026/0258353 A1
(43) Pub. Date: Sep. 3, 2026

(54) METHODS FOR THE INDUCTION OF TISSUE REGENERATION IN POSTMORTEM TISSUES

(71) Applicant: LifeCraft Sciences, Inc., Richmond, CA (US)

(72) Inventor: Michael D. WEST, Dowagiac, MI (US)

(73) Assignee: LifeCraft Sciences, Inc., Richmond, CA (US)

(21) Appl. No.: 19/555,405

(22) Filed: Mar. 3, 2026

Related U.S. Application Data
(60) Provisional application No. 63/766,077, filed on Mar. 3, 2025.

(51) Int. Cl. C12N 5/079; C07K 14/47; C12N 15/86; C12N 15/88

(52) U.S. Cl. CPC C12N 5/0621; C07K 14/4702; C12N 15/86; C12N 15/88; C12N 2510/00; C12N 2750/14143

(57) ABSTRACT

Methods and compositions are provided for the reprogramming of cells, tissues, and organs within postmortem metazoan animals. The methods include the use of vectors expressing factors capable of reversing the developmental aging of cells in a postmortem metazoan back to an embryonic regenerative state and the subsequent transplantation of cells, tissues, and organs from said postmortem animal into an allogeneic animal for therapeutic purposes or the subsequent resuscitation of said postmortem animal.

### BACKGROUND

Advances in stem cell technology, such as the isolation and propagation in vitro of human pluripotent stem (hPS) cells constitute an important area of medical research by providing methods for the manufacture of virtually any type of somatic cells for use in studying early embryonic development and for potential use in transplant medicine. Unlike adult- or even fetal-derived cells, hPS cell-derived cells manufactured in vitro typically display an immature pattern of gene expression even in the case where they are otherwise fully differentiated. This is observed, for example, in hPS cell-derived clonally isolated lines of human embryonic progenitor cells (hEPs). The clonal isolation and expansion of hEPs provides a means to propagate novel highly purified cell lineages with a prenatal, and typically even a pre-fetal pattern of gene expression. This immature (but nevertheless differentiated) phenotype is the basis of such cells displaying the potential for scarless regeneration in early development such as before the embryonic-fetal transition (EFT). Said cells and tissues are therefore useful for regenerating tissues such as, by way of nonlimiting example, the skin in a scarless manner. Such cell types have important applications in research, and for the manufacture of cell-based regenerative therapies (see PCT application No. PCT/US2006/013519 filed on Apr. 11, 2006 and titled “Novel Uses of Cells With Prenatal Patterns of Gene Expression” (WO2007058671A1); U.S. patent application Ser. No. 11/604,047 filed on Nov. 21, 2006 and titled “Methods to Accelerate the Isolation of Novel Cell Strains from Pluripotent Stem Cells and Cells Obtained Thereby” (US2008-0070303A1); and U.S. patent application Ser. No. 12/504,630 filed on Jul. 16, 2009 and titled “Methods to Accelerate the Isolation of Novel Cell Strains from Pluripotent Stem Cells and Cells Obtained Thereby” (US 2010-0184033 A1), each incorporated herein by reference). In contrast to embryonic cells (i.e. pluripotent stem cells and the immature cells derived in vitro from pluripotent stem cells capable of scarless regeneration), fetal and adult derived cells often show reduced potential for organogenesis in vitro and epimorphic scarless regeneration in vivo. Epimorphic regeneration, sometimes referred to as “epimorphosis,” refers to a type of tissue regeneration wherein a blastema of relatively undifferentiated mesenchyme proliferates at the site of injury followed by differentiation of said blastema cells to restore the original tissue histology.

Therefore, it is useful to distinguish the maturation state of cells from their differentiated state. Therefore, the term “maturation” as used in the present disclosure refers to the change in phenotype of somatic cells such that said cells no longer are capable of scarless (epimorphic) regeneration. In contrast, the term “differentiation” as used in the present invention refers to change of phenotype of cells from a state of pluripotency or multipotency to a state of reduced or absent multipotency wherein the cells take on individual characteristics of and reach their fetal or adult form and function. Therefore, cells may differentiate in vivo without maturing as in the case of animals capable of epimorphic regeneration. In the case of cells differentiated in vitro from pluripotent stem cells, these progeny may differentiate in recognizable cell types such as vascular endothelial cells or cardiomyocytes while not maturing to a non-regenerative scar-forming phenotype as evidenced by their continuing to express RNA markers of immature cells such as isoforms of the alpha and beta clustered protocadherin locus such as PCDHB2 and lack expression of fetal and adult markers such as COX7A1 and PCDHGA12.

The developmental timing of the loss of epimorphic potential cannot be fixed precisely, and likely varies with tissue type, nevertheless, the embryonic-fetal transition (EFT), or eight weeks of human development (Carnegie Stage 23; O'Rahilly, R., F. Miller (1987) Developmental Stages in Human Embryos, Including a Revision of Streeter's ‘Horizons’ and a Survey of the Carnegie Collection. Washington, Carnegie Institution of Washington) appears to temporally correspond to the loss of skin regeneration in placental mammals (Walmsley, G. G. et al 2015. Scarless Wound Healing: Chasing the Holy Grail Plast Reconstr Surg. 135(3): 907-17) and many other tissues as well. Some tissues, however, such as the mammalian heart, is capable of scarless regeneration for up to one week of postnatal development. Correlations between species show increased regenerative potential in the embryonic or larval state (reviewed in Morgan, T. H. (1901). Regeneration (New York: The MacMillan Company); also Sanchez Alvarado, A, and Tsonis, P. A. (2006). Bridging the regeneration gap: genetic insights from diverse animal models (Nat. Rev. Genet. 7, 873-884) suggest that tissue regeneration, as opposed to scarring, reflects the presence of an embryonic as opposed to fetal or adult phenotype. In the case of some species, a change in developmental timing (heterochrony) correlates with profound regenerative potential such as is the case in the developmental arrest in larval development (heterochrony) and limb regeneration observed in the Mexican salamander axolotl (A mexicanum) (Voss, S. R. et al, Thyroid hormone responsive QTL and the evolution of paedomorphic salamanders. Heredity (2012) 109, 293-298). In contrast, some animals such as the African Spiny mouse (Acomys cahirinus) show a profound potential for skin regeneration in the absence of overt heterochrony, perhaps reflecting uncharacterized molecular alterations (Gawriluk, T. R., 2016. Comparative analysis of ear-hole closure identifies epimorphic regeneration as a discrete trait in mammals. Nature Commun. 7:1 1164). We previously disclosed compositions and methods related to markers of the EFT in mammalian species and their use in modulating tissue regeneration (See, e.g. U.S. provisional patent application No. 61/831,421, filed Jun. 5, 2013, PCT patent application PCT/US2014/040601, filed Jun. 3, 2014 and U.S. patent application Ser. No. 14/896,664, filed on Dec. 7, 2015, (see PCT application Ser. No. PCT/US2019/028816 filed on Apr. 23, 2019 and titled “Improved Methods for Inducing Tissue Regeneration and Senolysis in Mammalian Cells”; U.S. published application no. 2020/0225213A1, filed on Jan. 7, 2020 and titled “Compositions and Methods for Detecting Cardiotoxicity”; U.S. published application no. 2020/0306296A1, filed on Mar. 27, 2020 and titled “Induced Tissue Regeneration Using Extracellular Vesicles”; U.S. published application no. 2022/0316013A1, filed on Aug. 25, 2020 and entitled “Differentially-Methylated Regions of the Genome Useful as Markers of Embryo-Adult Transitions”; U.S. provisional patent application No. 63/155,631, filed on Mar. 2, 2021 and titled “Use of Protocadherins in Methods of Diagnosing and Treating Cancer”; PCT application Ser. No. PCT/US2022/018563, filed on Mar. 2, 2022 and entitled “Methods and Compositions Used to Modify Chromatin Architecture to Regulate Phenotype in Aging and Cancer;” PCT application Ser. No. PCT/US2022/046737, filed on Oct. 14, 2022 and titled “Methods for the Temporal Regulation of Reprogramming Factors in Mammalian Cells”; U.S. provisional patent application No. 63/256,286, filed on Oct. 15, 2021 and titled “Methods for Modulating the Regenerative Phenotype in Mammalian Cells,” the disclosures of which are hereby incorporated by reference in their entirety.

Early candidates for regulators of heterochrony were identified in C. elegans. These included lin-28/let-7 (Ambros, V. and Horvitz, H. R. (1984). Heterochronic mutants of the nematode Caenorhabditis elegans. Science 226, 409-416). More recently, transgenic expression of the paralog Lin28a in mice has been reported to increase skin regeneration and amputated digit regrowth following wounding and to increase markers of oxidative phosphorylation (Shyh-Chang, N. et al 2013. Lin28 Enhances Tissue Repair by Reprogramming Cellular Metabolism. Cell 155, 778-792). However, the regenerative potential in said mice is not comparable to the profound epimorphosis observed in Acomys cahirinus. Despite these advances in regenerative medicine, the diverse applications of “partial reprogramming” synonymous with “induced tissue regeneration” or “i TR,” have only begun to be conceived or reduced to practice. Initial encouraging results indicating a reversal of developmental aging to reawaken the embryonic regenerative state have been obtained through the transient application of genes or gene products from the list: OCT4, SOX2, KLF4, MYC, LIN28A, and NANOG (designated herein as “global reprogramming factors’ given their potential to completely reprogram diverse somatic cell types back to pluripotency. The transient expression of global reprogramming factors carries the risk of over-reprogramming which could lead to the formation of benign teratomas. This would be a risk factor for their application in a clinical setting in a living patient. Therefore, “partial reprogramming” or what is herein designated “induced tissue regeneration (iTR) that precisely regulates the expression of global reprogramming factors or utilizes the critical downstream regulators of the regenerative state wherein said downstream regulators do not have the potential to reprogram cells to pluripotency (designated herein as “segmental reprogramming factors”) provide a significant improvement to ensure a safe application of the technology and reduce cancer risk. The present disclosure provides yet another method of providing cells and tissues capable of scarless regeneration. The present disclosure describes the novel application of global and segmental reprogramming methods to reverse developmental aging and induce a regenerative state in cells and tissues in whole animals in a postmortem state as well as the diverse compositions and methods resulting therefrom.

### SUMMARY

The present disclosure provides compounds, compositions, and methods useful for the reprogramming of populations of cells, tissues and organs in a postmortem metazoan animal, more preferably a postmortem mammalian body, and most preferably the postmortem human body, wherein the reprogramming reverses molecular developmental pathways such that the cells in the tissue and organ are reverted to a phenotype capable of scarless tissue regeneration wherein the metazoan body is in a postmortem state. Said reprogrammed tissues are useful for research in the biology of tissue regeneration; and for reprogramming cells, tissues, and organs for use in transplantation, wherein said reprogramming results in said cells, tissues, and organs demonstrating improved engraftment and function post-transplantation; as well as, for providing a method of resuscitation of a mammal in an otherwise terminal state, including a human. Said resuscitation is accomplished wherein the physiology of the mammal is stabilized by life support technologies including but not limited to hypothermia, mechanical ventilation, extracorporeal membrane oxygenation (ECMO), dialysis, cardiac assist devices, or the transient use of artificial organs, and the organs or the whole body are exposed to reprogramming factors to induce a regenerative state therein. Methods are also described herein where dysfunctional cells, tissues and organs in the postmortem metazoan are decellularized, while providing a three-dimensional matrix of said cells, tissue or organ, thereby facilitating subsequent tissue regeneration while the metazoan body i-His stabilized by life support technologies.

In one aspect of the present disclosure, methods are provided for reprogramming one or more cell, tissue, or organ of a postmortem metazoan animal, preferably a mammal, most preferably a human having cellular necrosis of the one or more cell, tissue or organ, to a regenerative state without inducing pluripotency of the one or more cell, tissue or organ, comprising the steps of: stabilizing the physiology of the metazoan animal using one or more life support technology; exposing the one or more cell, tissue or organ to one or more induced tissue regeneration (iTR) factor gene, encoded iTR RNA and/or expressed iTR protein; wherein the regenerative state is demonstrated by the ability of the one or more cell, tissue or organ to exhibit scarless regeneration; wherein the iTR gene, iTR RNA and/or iTR protein is capable under other conditions of inducing pluripotency in the one or more cell, tissue or organ; wherein the one or more iTR factor gene, encoded iTR RNA and/or expressed iTR protein is selected from the group of iTR genes consisting of OCT4, SOX2, KLF4, NANOG, ESRRB, NR5A2, CEBPA, MYC, LIN28A, TERT, and LIN28B, and/or their encoded iTR RNAs and iTR protein.

In another embodiment, methods are provided for reprogramming one or more cell, tissue, or organ of a postmortem metazoan animal, preferably a mammal, most preferably a human having cellular necrosis of the one or more cell, tissue or organ, to a regenerative state without inducing pluripotency of the one or more cell, tissue or organ, comprising the steps of: stabilizing the physiology of the metazoan animal using one or more life support technology; exposing the one or more cell, tissue or organ to one or more induced tissue regeneration (iTR) factor gene, encoded iTR RNA and/or expressed iTR protein; wherein the regenerative state is demonstrated by the ability of the one or more cell, tissue or organ to exhibit scarless regeneration; wherein the iTR gene, iTR RNA and/or iTR protein is capable under other conditions of inducing pluripotency in the one or more cell, tissue or organ; wherein the one or more iTR factor gene, encoded iTR RNA and/or expressed iTR protein is selected from the group of iTR genes consisting of IGF2BP1, IGF2BP2, IGF2BP3, LIN28A, HMGA2, H19, FIRRE, LIN28B, or TERT.

In another embodiment, methods are provided for reprogramming one or more cell, tissue, or organ of a postmortem metazoan animal, preferably a mammal, most preferably a human having cellular necrosis of the one or more cell, tissue or organ, to a regenerative state without inducing pluripotency of the one or more cell, tissue or organ, comprising the steps of: stabilizing the physiology of the metazoan animal using one or more life support technology; exposing the one or more cell, tissue or organ to one or more induced tissue regeneration (iTR) factor gene, encoded iTR RNA and/or expressed iTR protein; wherein the regenerative state is demonstrated by the ability of the one or more cell, tissue or organ to exhibit scarless regeneration; wherein the iTR gene, iTR RNA and/or iTR protein is capable under other conditions of inducing pluripotency in the one or more cell, tissue or organ; wherein the one or more iTR factor gene, encoded iTR RNA and/or expressed iTR protein is selected from the group of iTR genes consisting of WTAP, METTL3, METTL14, TERT and the metabolite L-2-HG, RNAi directed to the eraser FTO, and/or molecules inactivating IGFBP6 such as monoclonal antibody-based competitive inhibition, or a combination of the aforementioned factors, thereby inducing a regenerative phenotype in the cells or the corresponding tissues.

In another embodiment, methods are provided for reprogramming one or more cell, tissue, or organ of a postmortem metazoan animal, preferably a mammal, most preferably a human having cellular necrosis of the one or more cell, tissue or organ, to a regenerative state without inducing pluripotency of the one or more cell, tissue or organ, comprising the steps of: stabilizing the physiology of the metazoan animal using one or more life support technology; exposing the one or more cell, tissue or organ to one or more induced tissue regeneration (iTR) factor gene, encoded iTR RNA and/or expressed iTR protein; wherein the regenerative state is demonstrated by the ability of the one or more cell, tissue or organ to exhibit scarless regeneration; wherein the iTR gene, iTR RNA and/or iTR protein is capable under other conditions of inducing pluripotency in the one or more cell, tissue or organ; wherein the one or more iTR factor gene, encoded iTR RNA and/or expressed iTR protein is selected from the group of iTR genes consisting of OCT4, SOX2, KLF4, NANOG, ESRRB, NR5A2, CEBPA, MYC, LIN28A, TERT, and LIN28B, IGF2BP1, IGF2BP2, IGF2BP3, HMGA2, H19, FIRRE, WTAP, METTL3, METTL14, TERT and the metabolite L-2-HG, RNAi directed to the eraser FTO, and/or molecules inactivating IGFBP6 such as monoclonal antibody-based competitive inhibition, or a combination of the aforementioned factors, thereby inducing a regenerative phenotype in the cells or the corresponding tissues and/or their encoded iTR RNAs and iTR protein.

In one aspect of the present disclosure, methods are provided for enhancing scarless tissue regeneration in a non-viable metazoan animal with significant but not total cell necrosis throughout the organism comprised of the steps of I) stabilization of the physiology of the metazoan body using life support technology; 2) exposure of a population of cells, a tissue or an organ in the body to factors capable of reprogramming the cells tissue or organ to a state of induced tissue regeneration, but not pluripotency, said factors including one or more of the genes: OCT4, SOX2, KLF4, NANOG, ESRRB, NR5A2, CEBPA, MYC, LIN28A, TERT, and LIN28B, their encoded RNAs, or expressed proteins, and including genetically-modified versions of the aforementioned genes, including, but not limited to modified SOX2, as described (in Maccarthy, Caitlin M. et al, “Highly cooperative chimeric super-SOX induces naive pluripotency across species,” Cell Stem Cell, Volume 31, Issue 1, 127-147.e9t 3) monitoring the regeneration of said tissue until it reaches a state of regeneration capable of sustaining independent life, and 4) removing said body from life support technology and resuscitating the organism.

In one aspect of the present disclosure, methods are provided for enhancing scarless tissue regeneration in a non-viable metazoan animal with significant but not total cell necrosis throughout the organism comprised of the steps of 1) stabilization of the physiology of the metazoan body using life support technology; 2) exposure of a population of cells, a tissue or an organ in the body to factors capable of reprogramming the cells tissue or organ to a state of induced tissue regeneration, but not pluripotency, said factors including one or more of the genes: OCT4, SOX2, NANOG, and LIN28A, their encoded RNAs, or expressed proteins, and including genetically-modified versions of the aforementioned genes, including, but not limited to modified SOX2, as described (in Maccarthy, Caitlin M. et al, “Highly cooperative chimeric super-SOX induces naive pluripotency across species,” Cell Stem Cell, Volume 31, Issue 1, 127-147.e9t 3) monitoring the regeneration of said tissue until it reaches a state of regeneration capable of sustaining independent life, and 4) removing said body from life support technology and resuscitating the organism.

In one aspect of the present disclosure, methods are provided for enhancing scarless tissue regeneration in a non-viable metazoan animal with significant but not total cell necrosis throughout the organism comprised of the steps of 1) stabilization of the physiology of the metazoan body using life support technology; 2) exposure of a population of cells, a tissue or an organ in the body to factors capable of reprogramming the cells tissue or organ to a state of induced tissue regeneration, but not pluripotency, said factors including the genes: OCT4, SOX2, KLF4, and MYC, their encoded RNAs, or expressed proteins, and including genetically-modified versions of the aforementioned genes, including, but not limited to modified SOX2, as described (in Maccarthy, Caitlin M. et al, “Highly chimeric super-SOX induces naive pluripotency across species,” Cell Stem Cell, Volume 31, Issue 1, 127-147.e9t; 3) monitoring the regeneration of said tissue until it reaches a state of regeneration capable of sustaining independent life, and 4) removing said body from life support technology and resuscitating the organism.

In another aspect of the present disclosure, methods are provided for enhancing scarless tissue regeneration in a non-viable mammal with significant but not total cell necrosis throughout the organism comprised of the steps of 1) stabilization of the physiology of the mammalian body using life support technology; 2) exposure of a population of cells, a tissue or an organ in the body to factors capable of reprogramming the population of cells, at issue or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of the aforementioned genes including but not limited to modified SOX2, as described (in Maccarthy, Caitlin M. et al, “Highly cooperative chimeric super-SOX induces naive pluripotency across species,” Cell Stem Cell, Volume 31, Issue 1, 127-147.e9t; 3) monitoring the regeneration of said population of cells, tissue or an organ until it reaches a state of regeneration capable of sustaining independent life, and 4) removing said mammal from life support technology and resuscitating the mammal.

In another aspect of the present disclosure, methods are provided for enhancing scarless tissue regeneration in a non-viable mammal with significant but not total cell necrosis throughout the organism comprised of the steps of 1) stabilization of the physiology of the cells in mammalian body by means of reducing the animal's body temperature while providing ventilation support; 2) exposure of a population of cells, a tissue or an organ in the body to factors capable of reprogramming the population of cells, tissue or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2, as described fin Maccarthy, Caitlin M. et al, “Highly cooperative chimeric super-SOX induces naive pluripotency across species,” Cell Stem Cell, Volume 31, Issue 1, 127-147.e9; 3) monitoring the regeneration of said population of cells, tissue or an organ until it reaches a state of regeneration capable of sustaining independent life, and 4) removing said mammal from life support technology and resuscitating the mammal. In another aspect of the present disclosure, methods are provided for enhancing scarless tissue regeneration in a non-viable postmortem human with significant but not total cell necrosis throughout the body comprised of the steps of 1) stabilization of the physiology of the human body using life support technology, 2) exposure of a population of cells, a tissue or an organ in the body to factors capable of reprogramming the population of cells, a tissue or an organ o a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described (in Maccarthy, Caitlin M. et al, “Highly cooperative chimeric super-SOX induces naive pluripotency across species,—” Cell Stem Cell, Volume 31, Issue 1, 127-147.e9; 3) monitoring the regeneration of said population of cells a tissue or an organ until it reaches a state of regeneration capable of sustaining independent life, and 4) removing said human from life support technology and resuscitating the human.

In another aspect of the present disclosure, methods are provided for enhancing scarless tissue regeneration in a non-viable postmortem cryopreserved metazoan with significant but not total cell necrosis throughout the body comprised of the steps of 1) raising the temperature of said metazoan above 0.0 degrees Centigrade; 2) stabilization of the physiology of the metazoan body using life support technology; 3) exposure of a population of cells, a tissue or an organ in the body to factors capable of reprogramming the population of cells, a tissue or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9t; 4) monitoring the regeneration of said population of cells, a tissue, or an organ until it reaches a state of regeneration capable of sustaining independent life, and 5) removing said metazoan from life support technology and resuscitating the metazoan.

In another aspect of the present disclosure, methods are provided for enhancing scarless tissue regeneration in a non-viable postmortem cryopreserved mammal with significant but not total cell necrosis throughout the body comprised of the steps of 1) raising the temperature of said mammal above 0.0 degrees Centigrade, 2) stabilization of the physiology of the mammalian body using life support technology, 3) exposure of a population of cells, a tissue, or an organ in the body to factors capable of reprogramming the population of cells, a tissue, or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2, as described in Maccarthy, Caitlin M. et al, “Highly cooperative chimeric super-SOX induces naive pluripotency across species,” Cell Stem Cell, Volume 31, Issue 1, 127-147.e9t; 4) monitoring the regeneration of said population of cells, a tissue or an organ until it reaches a state of regeneration capable of sustaining independent life, and 5) removing said mammal from life support technology and resuscitating the mammal.

In another aspect of the present disclosure, methods are provided for enhancing scarless tissue regeneration in a non-viable postmortem cryopreserved human with significant but not total cell necrosis throughout the body comprised of the steps of 1) raising the temperature of said human above 0.0 degrees Centigrade, 2) stabilization of the physiology of the human body using life support technology, 3) exposure of a population of cells, a tissue, or an organ in the body to factors capable of reprogramming the population of cells, a tissue, or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said population of cells, a tissue or an organ until it reaches a state of regeneration capable of sustaining independent life, and 5) removing said human from life support technology and resuscitating the human.

In another aspect of the present disclosure, methods are provided for enhancing scarless tissue regeneration in an otherwise non-viable metazoan animal population of cells, a tissue, or an organ following thawing from a cryopreserved state with significant but not total cell necrosis throughout the population of cells, a tissue or an organ comprised of the steps of 1) raising the temperature of said metazoan population of cells, a tissue, or an organ above 0.0 degrees Centigrade, 2) stabilization of the physiology of the metazoan population of cells, a tissue, or an organ using tissue culture support technology, 3) ex vivo exposure of a population of cells, a tissue, or an organ in the body to factors capable of reprogramming the population of cells, a tissue, or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said population of cells, a tissue, or an organ until it reaches a state of regeneration capable of functioning when transplanted into a similar metazoan animal, and 5) transplanting said population of cells, a tissue, or an organ into a similar organism to study regeneration or to perform transplant therapy.

In another aspect of the present disclosure, methods are provided for enhancing scarless tissue regeneration in an otherwise non-viable mammalian animal population of cells, a tissue, or an organ following thawing from a cryopreserved state with significant but not total cell necrosis throughout the population of cells, a tissue, or an organ comprised of the steps of 1) raising the temperature of said mammalian population of cells, a tissue, or an organ above 0.0 degrees Centigrade, 2) stabilization of the physiology of the mammalian tissue using tissue culture support technology, 3) ex vivo exposure of a tissue in the body to factors capable of reprogramming the cells to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said tissue until it reaches a state of regeneration capable of functioning when transplanted into a similar mammalian animal, and 5) transplanting said tissue into a similar organism to study regeneration or to perform transplant therapy.

In another aspect of the present disclosure, methods are provided for enhancing scarless tissue regeneration in an otherwise non-viable human tissue following thawing from a cryopreserved state with significant but not total cell necrosis throughout the tissue comprised of the steps of 1) raising the temperature of said human tissue above 0.0 degrees Centigrade, 2) stabilization of the physiology of human tissue using tissue culture support technology, 3) ex vivo exposure of a tissue in the body to factors capable of reprogramming the cells to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said population of cells, a tissue, or an organ until it reaches a state of regeneration capable of functioning when transplanted into another human, and 5) transplanting said population of cells, a tissue, or an organ into another human to study regeneration or to perform transplant therapy.

In another aspect of the present disclosure, methods are provided for enhancing scarless tissue regeneration in an otherwise non-viable metazoan animal population of cells, a tissue, or an organ with significant but not total cell necrosis throughout the population of cells, a tissue, or an organ comprised of the steps of 1) stabilization of the physiology of the metazoan population of cells, a tissue, or an organ, 2) Decellularization of the diseased or damaged part of the population of cells, a tissue, or an organ, 3) exposure of the population of cells, a tissue, or an organ to factors capable of reprogramming the population of cells, a tissue, or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2, as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said tissue until it reaches a state of regeneration capable of normal function, and 5) transplanting said population of cells, a tissue, or an organ into another non-viable metazoan animal to study regeneration or to perform transplant therapy.

In another aspect of the present disclosure, methods are provided for enhancing scarless tissue regeneration in an otherwise non-viable mammalian tissue or organ with significant but not total cell necrosis throughout the tissue comprised of the steps of 1) stabilization of the physiology of the mammalian tissue, 2) Decellularization of the diseased or damaged part of the tissue or organ, 3) exposure of the tissue or organ to factors capable of reprogramming the cells to a state of induced tissue regeneration but not pluripotency said factors including one or more of the genes: OCT4, SOX2, KLF4, NANOG, ESRRB, NR5A2, CEBPA, MYC, LIN28A, TERT, amylin, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said tissue until it reaches a state of regeneration capable of normal function; and transplanting said tissue into another non-viable mammal to study regeneration or to perform transplant therapy.

In another aspect of the present disclosure, methods are provided for enhancing scarless tissue regeneration in an otherwise non-viable human animal tissue or organ with significant but not total cell necrosis throughout the tissue comprised of the steps of 1) stabilization of the physiology of the human tissue or organ, 2) Decellularization of the diseased or damaged part of the tissue or organ, 3) exposure of the tissue or organ to factors capable of reprogramming the cells to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said tissue until it reaches a state of regeneration capable of normal function.

In another aspect of the present disclosure, methods are provided for enhancing scarless tissue regeneration in a metazoan animal population of cells, a tissue, or an organ containing cancer cells comprised of the steps of 1) stabilization of the physiology of the metazoan population of cells, a tissue, or an organ, 2) Decellularization of the portion of the population of cells, a tissue, or an organ containing cancer cells, 3) exposure of the population of cells, a tissue, or an organ to factors capable of reprogramming the cells to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said population of cells, a tissue, or an organ until it reaches a state of regeneration capable of normal function without cancer.

In another aspect of the present disclosure, methods are provided for enhancing scarless tissue regeneration in a mammalian population of cells, a tissue, or an organ containing cancer cells comprised of the steps of 1) stabilization of the physiology of the mammalian population of cells, a tissue, or an organ, 2) Decellularization of the portion of the population of cells, a tissue, or an organ containing cancer cells, 3) exposure of the population of cells, a tissue, or an organ to factors capable of reprogramming the population of cells, a tissue, or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2, as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said population of cells, a tissue, or an organ until it reaches a state of regeneration capable of normal function without cancer.

In another aspect of the present disclosure, methods are provided for enhancing scarless tissue regeneration in a mammalian population of cells, a tissue, or an organ containing cancer cells comprised of the steps of 1) stabilization of the physiology of the mammalian population of cells, a tissue, or an organ, 2) exposure of the animal, population of cells, a tissue, or an organ to factors capable of reprogramming the population of cells, a tissue, or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 3) monitoring the reprogramming of said postmortem animal population of cells, a tissue, or an organ in the animal are reprogrammed to pluripotency with resulting teratoma formation; and 4) harvesting said teratomas for population of cells, a tissue, or an organ capable of being used in transplantation with improved potential for scarless regeneration.

In another aspect of the present disclosure, methods are provided for enhancing scarless tissue regeneration in a mammalian population of cells, a tissue, or an organ containing cancer cells comprised of the steps of 1) stabilization of the physiology of the mammalian animal population of cells, a tissue, or an organ, 2) exposure of the animal, population of cells, a tissue, or an organ to iPS cells generated from a patient in need of transplantable population of cells, a tissue, or an organ; 3) monitoring the growth of resulting teratoma formation; and 4) harvesting said teratomas for population of cells, a tissue, or an organ capable of being used in transplantation with improved potential for scarless regeneration.

In another aspect of the present disclosure, methods are provided for enhancing scarless tissue regeneration in a mammalian population of cells, a tissue, or an organ containing cancer cells comprised of the steps of I) stabilization of the physiology of the mammalian animal population of cells, a tissue, or an organ, 2) exposure of the animal, population of cells, a tissue, or an organ to ES cells genetically modified to reduced immunotolerance of said population of cells, a tissue, or an organ when transplanted in allogeneic animals; 3) monitoring the growth of resulting teratoma formation; and 4) harvesting said teratomas for population of cells, a tissue, or an organ capable of being used in transplantation with improved potential for scarless regeneration.

In another aspect of the present disclosure, methods are provided for enhancing scarless tissue regeneration in a human population of cells, a tissue, or an organ containing cancer cells comprised of the steps of I) stabilization of the physiology of the human population of cells, a tissue, or an organ, 2) Decellularization of the portion of the population of cells, a tissue, or an organ containing cancer cells, 3) exposure of the population of cells, a tissue, or an organ to factors capable of reprogramming the population of cells, a tissue, or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 4) monitoring the regeneration of said population of cells, a tissue, or an organ until it reaches a state of regeneration capable of normal function without cancer.

In another aspect of the present disclosure, methods are provided for enhancing scarless tissue regeneration in a postmortem metazoan population of cells, a tissue, or an organ containing cancer cells comprised of the steps of 1) stabilization of the physiology of the metazoan animal population of cells, a tissue, or an organ, 2) exposure of the population of cells, a tissue, or an organ to factors capable of reprogramming the population of cells, a tissue, or an organ to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,—” Cell Stem Cell, Volume 31, Issue 1, 127-147.e9; 3) monitoring the reprogramming of said population of cells, a tissue, or an organ such as by measuring the transcription of gene markers of regeneration such as those differentially regulated in early embryonic development compared to the adult state including but not limited to Cox7a1, Pcdhgal 2, Comt, Adirf, or Naaladll; 4) harvesting said cells, tissue, or organ for use in transplantation; and 5) transplantation of said cells, tissue, or organ with potential for scarless regeneration into an allogeneic animal.

In another aspect of the present disclosure, methods are provided for enhancing scarless tissue regeneration in a postmortem mammalian population of cells, a tissue, or an organ containing cancer cells comprised of the steps of 1) stabilization of the physiology of the mammal by means of life support technology, 2) exposure of the population of cells, a tissue, or an organ to factors capable of reprogramming the cells to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 3) monitoring the reprogramming of said cells, tissue, or organ such as by measuring the transcription of gene markers of regeneration such as those differentially regulated in early embryonic development compared to the adult state including but not limited to COX7A1, PCDHGA12, COMT, ADIRF, or NMLADLJ; 4) harvesting said population of cells, a tissue, or an organ for use in transplantation; and 5) transplantation of said population of cells, a tissue, or an organ with potential for scarless regeneration into an allogeneic animal.

In another aspect of the present disclosure, methods are provided for enhancing scarless tissue regeneration in a postmortem human tissue or organ containing cancer cells comprised of the steps of 1) stabilization of the physiology of the human by life support technology, 2) exposure of the population of cells, a tissue, or an organ to factors capable of reprogramming the cells to a state of induced tissue regeneration but not pluripotency said factors including one or more of the genes: OCT4, SOX2, KLF4, NANOG, ESRRB, NR5A2, CEBPA, MYC, LIN28A, TERT, and LIN28B, their encoded RNAs, or proteins, and including genetically-modified versions of aforementioned genes including but not limited to modified SOX2 as described in Maccarthy, Caitlin M. et al, “Highly cooperative chimeric super-SOX induces naive pluripotency across species,” Cell Stem Cell, Volume 31, Issue 1, 127-147.e9; 3) monitoring the reprogramming of said cells, tissue, or organ such as by measuring the transcription of gene markers of regeneration such as those differentially regulated in early embryonic development compared to the adult state including but not limited to COX7A1, PCDHGA12, COMT, ADIRF, or NAALADLJ; 4) harvesting said population of cells, a tissue, or an organ for use in transplantation; and 5) transplantation of said population of cells, a tissue, or an organ with potential for scarless regeneration into an allogeneic recipient.

In another aspect of the present disclosure, methods are provided for obtaining tissue suitable for use in transplantation therapy, said method comprised of the steps of 1) stabilization of the physiology of a postmortem human by means of life support technology, 2) exposure of the population of cells, a tissue, an organ, or the entire body to factors capable of reprogramming the population of cells, a tissue, or an organ to a state of pluripotency said factors including one or more of the genes: OCT4, SOX2, KLF4, NANOG, ESRRB, NR5A2, CEBPA, MYC, LIN28A, TERT, and LIN28B, their encoded RNAs 3) maintain said mammal on life support until teratomas appear, 3) surgically-excising said teratomas to obtain cells, tissue, and organs useful in transplantation therapy to a living human being.

Table I. The Pri- Pre- and mature miRNA sequences of the LET-7 family members described in the instant application.

TABLE I
LET-7 Family Members (sharing the LET-7 seed sequence)

| A. Gene Name | Position hg38 |
|---|---|
| MIRLET7A1 | chr9: 94, 175, 957-94, 176, 036 |
| MIRLET7A2 | chr11: 122, 146, 522-122, 146, 593 |
| MIRLET7A3 | chr22: 46, 112, 749-46, 112, 822 |
| MIRLET7B | chr22: 46, 113, 686-46, 113, 768 |
| MIRLET7C | chr21: 16, 539, 828-16, 539, 911 |
| MIRLET7D | chr9: 94, 178, 834-94, 178, 920 |
| MIRLET7E | chr19: 51, 692, 786-51, 692, 864 |
| MIRLET7F1 | chr9: 94, 176, 347-94, 176, 433 |
| MIRLET7F2 | chrX: 53, 557, 192-53, 557, 274 |
| MIRLET7G | chr3: 52, 268, 278-52, 268, 361 |
| MIR98 | chrX: 53, 557, 191-53, 557, 271 |
| MIR4458 | chr5: 8, 460, 925-8, 460, 999 |
| MIR4500 | chr13: 87, 618, 665-87, 618, 740 |

### Primary Transcript (Pri-miRNA) Designations

### B. Pri-Let-7 Transcripts

1. pri-MIRLET7A1
2. pri-MIRLET7A2
3. pri-MIRLET7A3
4. pri-MIRLET7B
5. pri-MIRLET7C
6. pri-MIRLET7D
7. pri-MIRLET7E
8. pri-MIRLET7F1
9. pri-MIRLET7F2
10. pri-MIRLET7G
11. pri-MIRLET7I

pri-Related Transcripts also designated herein as members of the LET-7 Family since they share a seed sequence
12. pri-MIR98
13. pri-MIR4458
14. pri-MIR-4500

### C. Pre-miRNA (Hairpin) Designations

### Canonical Let-7 Family (Human)

1. MIRLET7A1—hsa-mir-let-7a-1
2. MIRLET7A2—hsa-mir-let-7a-2
3. MIRLET7A3—hsa-mir-let-7a-3
4. MIRLET7B—hsa-mir-let-7b
5. MIRLET7C—hsa-mir-let-7c
6. MIRLET7D—hsa-mir-let-7d
7. MIRLET7E—hsa-mir-let-7e
8. MIRLET7F1—hsa-mir-let-7f-1
9. MIRLET7F2—hsa-mir-let-7f-2
10. MIRLET7G—hsa-mir-let-7g
11. MIRLET7I—hsa-mir-let-7i

### Let-7—Related/Seed-Sharing Family Members

12. MIR98—hsa-mir-98
13. MIR4458—hsa-mir-4458
14. MIR-4500—has-mir-4500
   D. Mature miRNA Designations

Products derived from 5′ and/or 3′ arms of the precursor hairpin)

### Canonical Let-7 Members

hsa-let-7a-5p
hsa-let-7b-5p
hsa-let-7a-3p
hsa-let-7b-3p
hsa-let-7c-5p
hsa-let-7c-3p
hsa-let-7d-5p
hsa-let-7d-3p
hsa-let-7e-5p
hsa-let-7e-3p
hsa-let-7f-5p
hsa-let-7f-3p
hsa-let-7g-5p
hsa-let-7g-3p
hsa-let-7i-5p
hsa-let-7i-3p

Related Members considered part of the LET-7 family herein since they share the same seed sequence:
hsa-miR-98-5p
hsa-miR-98-3p
hsa-miR-4458-5p
hsa-miR-4458-3p
hsa-miR-4500-5p
hsa-miR-4500-3p

TABLE II
The 3′ UTR of LIN28A showing the target seed sequences to be deleted
or modified in order to prevent binding with the LET-7
family of miRNAs as described herein. Target seed
sequence positions are listed as well as positions within in the
entire sequence of the 3′ UTR where they are shown in bold italics.

| | | | |
|---|---|---|---|
| miRNA Name: | LET-7 Family | | |
| Seed Location | 905 | | |
| NCBI Gene ID | 79727 | GenBank Accession | NM_024674 |
| Gene Symbol | LIN28A | 3′ UTR Length | 3270 |
| Gene Description | lin-28 homolog A | | |

```
3′ UTR Sequence
      1 gccacaatgg gtgggggcta ttcttttgct atcaggaagt tttgaggagc aggcagagtg
    61 gagaaagtgg gaatagggtg cattggggct agttggcact gccatgtatc tcaggcttgg
  121 gttcacacca tcaccctttc ttccctctag gtggggggaa agggtgagtc aaaggaactc
  181 caaccatgct ctgtccaaat gcaagtgagg gttctggggg caaccaggag gggggaatca
  241 ccctacaacc tgcatacttt gagtctccat ccccagaatt tccagctttt gaaagtggcc
  301 tggataggga agttgttttc cttttaaaga aggatatata ataattccca tgccagagtg
  361 aaatgattaa gtataagacc agattcatgg agccaagcca ctacattctg tggaaggaga
  421 tctctcagga gtaagcattg tttttttttc acatcttgta tcctcatacc cacttttggg
  481 atagggtgct ggcagctgtc ccaagcaatg ggtaatgatg atggcaaaaa gggtgtttgg
  541 gggaacagct gcagacctgc tgctctatgc tcacccccgc cccattctgg gccaatgtga
  601 ttttatttat ttgctccctt ggatactgca ccttgggtcc cactttctcc aggatgccaa
  661 ctgcactagc tgtgtgcgaa tgacgtatct tgtgcatttt aacttttttt ccttaatata
  721 aatattctgg ttttgtattt ttgtatattt taatctaagg ccctcatttc ctgcactgtg
  781 ttctcaggta catgagcaat ctcagggata gccagcagca gctccaggtc tgcgcagcag
  841 gaattacttt ttgttgtttt tgccaccgtg gagagcaact atttggagtg cacagcctat
  901 tgaactacct catttttgcc aataagagct ggcttttctg ccatagtgtc ctcttgaaac
  961 cccctctgcc ttgaaaatgt tttatgggag actaggtttt aactgggtgg ccccatgact
1021 tgattgcctt ctactggaag attgggaatt agtctaaaca ggaaatggtg gtacacagag
1081 gctaggagag gctgggcccg gtgaaaaggc cagagagcaa gccaagatta ggtgagggtt
1141 gtctaatcct atggcacagg acgtgcttta catctccaga tctgttcttc accagattag
1201 gttaggccta ccatgtgcca cagggtgtgt gtgtgtttgt aaaactagag ttgctaagga
1261 taagtttaaa gaccaatacc cctgtactta atcctgtgct gtcgagggat ggatatatga
1321 agtaaggtga gatccttaac ctttcaaaat tttcgggttc cagggagaca cacaagcgag
1381 ggttttgtgg tgcctggagc ctgtgtcctg ccctgctaca gtagtgatta atagtgtcat
1441 ggtagctaaa ggagaaaaag ggggtttcgt ttacacgctg tgagatcacc gcaaacctac
1501 cttactgtgt tgaaacggga caaatgcaat agaacgcatt gggtggtgtg tgtctgatcc
1561 tgggttcttg tctcccctaa atgctgcccc ccaagttact gtatttgtct gggctttgta
1621 ggacttcact acgttgattg ctaggtggcc tagtttgtgt aaatataatg tattggtctt
1681 tctccgtgtt ctttgggggt tttgtttaca aacttctttt tgtattgaga gaaaaatagc
1741 caaagcatct ttgacagaag gttctgcacc aggcaaaaag atctgaaaca ttagtttggg
1801 gggccctctt cttaaagtgg ggatcttgaa ccatcctttc ttttgtattc cccttcccct
1861 attacctatt agaccagatc ttctgtccta aaaacttgtc ttctaccctg ccctcttttc
1921 tgttcacccc caaaagaaaa cttacacacc cacacacata cacatttcat gcttggagtg
1981 tctccacaac tcttaaatga tgtatgcaaa aatactgaag ctaggaaaac cctccatccc
2041 ttgttcccaa cctcctaagt caagaccatt accatttctt tctttctttt tttttttttt
2101 ttaaaatgga gtctcactgt gtcacccagg ctggagtgca gtggcatgat cggctcactg
2161 cagcctctgc ctcttgggtt caagtgattc tcctgcctca gcctcctgag tagctgggat
2221 ttcaggcacc cgccacactc agctaatttt tgtattttta gtagagacgg ggtttcacca
2281 tgttgtccag gctggtctgg aactcctgac ctcaggtgat ctgcccacct tggcttccca
2341 aagtgctggg attacaggca tgagccacca tgctgggcca accatttctt ggtgtattca
2401 tgccaaacac ttaagacact gctgtagccc aggcgcggtg gctcacacct gtaatcccag
2461 cactttggaa ggctgaggcg ggcggatcac aaggtcacga gttcaaaact atcctggcca
2521 acacagtgaa accccgtctc tactaaaata caaaaaaatt agccgggtgt ggtggtgcat
2581 gcctttagtc ctagctattc aggaggctga ggcaggggaa tcgcttgaac ccgagaggca
2641 gaggttgcag tgagctgaga tcgcaccact gcactccagc ctggttacag agcaagactc
2701 tgtctcaaac aaaacaaaac aaaacaaaaa cacactactg tattttggat ggatcaaacc
2761 tccttaattt taatttctaa tcctaaagta aagagatgca attgggggcc ttccatgtag
2821 aaagtggggt caggaggcca agaaagggaa tatgaatgta tatccaagtc actcaggaac
2881 ttttatgcag gtgctagaaa ctttatgtca aagtggccac aagattgttt aataggagac
2941 gaacgaatgt aactccatgt ttactgctaa aaaccaaagc tttgtgtaaa atcttgaatt
3001 tatggggcgg gagggtagga aagcctgtac ctgtctgttt ttttcctgat ccttttccct
3061 cattcctgaa ctgcaggaga ctgagcccct ttgggctttg gtgaccccat cactggggtg
3121 tgtttatttg atggttgatt ttgctgtact gggtacttcc tttcccattt tctaatcatt
3181 ttttaacaca agctgactct tcccttccct tctcctttcc ctgggaaaat acaatgaata
3241 aataaagact tattggtacg caaactgtca
```

TABLE III
The sequence of the 3′ UTR of LIN28B showing the target seed sequences
to be deleted or modified in order to prevent binding with the
LET-7 family of miRNAs as described herein. Target seed sequence
positions are listed as well as positions within in the entire
sequence of the 3′ UTR where they are shown in bold italics.

| | | | |
|---|---|---|---|
| miRNA Name: | LET-7 Family | | |
| Seed Locations | 44-51, 2471-2477, | | |
| | 2653-2660, | | |
| | 4092-4098, | | |
| | 4277-4283 | | |
| NCBI Gene ID | 389421 | GenBank Accession | NM_001004317 |
| Gene Symbol | LIN28B | 3′ UTR Length | 4561 |
| Gene Description | lin-28 homolog B | | |

```
3′ UTR Sequence
      1 caggtcttct tcatatgttc tttcctttac ccggttgcaa agtctacctc atgcaagtat
    61 aggggaacag tatttcacaa gcagtagctg acctgggatt ttaactacta ttggggaact
  121 gtgaattttt taaacagaca aatcactcta agcaaattac atttgagcag ggtgtcatgt
  181 tttatgttaa ttcagagaat aagatactat gtctgtcaat atgtgcatgt gtgagaggga
  241 gagagcctga gtctgtgtgt gtacatgagg atttttatat aggaatgtag acacatatat
  301 aaagaggctt tgtctttata tatttgtgta tagatcaaag cacacaccct ctctcatata
  361 attggatatt tccaagaatt gaaaacccat gtgaagcatt atagatagtt ttaaatttaa
  421 cccactggag ttttcttgaa ataccacttc ttttatatta tataaaacta aaaacacgac
  481 tgttaccttt tgtgtgaacc aaaggatact tcagatctca gagctgccaa ttatggggta
  541 ctaaaggttt ttaagacatc cagttctccc gaatttggga ttgcctcttt ttcttgaaat
  601 ctctggagta gtaatttttt tccccctttt ttgaaggcag taccttaact tcatatgcct
  661 ctgactgcca taagcttttt tgattctggg ataacataac tccagaaaag acaatgaatg
  721 tgtaatttgg gccgatattt cactgtttta aattctgtgt ttaattgtaa aattagatgc
  781 ctattaagag aaatgaaggg gaggatcatc ttagtggctt gttttcagta gtattttaat
  841 atcagcttct tgtaaccttt tccatgttgt gagggttgta agggattgtg tggcaacagc
  901 agcttccctt ggctaactca atcttctacc cattgcttag agcagggagc cctccttatt
  961 tactactgaa gaccttagag aactccaatt gtttggcata tatttttggt ggtggttttt
1021 attcctcctg gagagttatc taatttgttt ctaaaacaaa caagcagcaa agaaatgaat
1081 taaatactgg ggttgagaat taaaattaag tggatgttca cagttgccca atatatatga
1141 cctgcaaatg atacgaaaaa gtgcagcatt tagtggcagt taacaagagt gacaagcctg
1201 gggcagaggt accaaacctc tcccaccaga gagctagaag tattttatac agtaactttg
1261 atcttatgga agtgaccttc aatgcttatt ctgaagtaac ctatatggtg gatacaggat
1321 gaacattcag tgccagggag aatcttctca ggttggttct cgttagagtg ataaactggc
1381 taggggccat agtattggtc ctgttaggtt tcggtcatgg aaaaaaaaat tattttgggg
1441 tcatcctggc tctagatgtt atgggcaaat ttctgaaaca tctgcaagaa ggtaccagtt
1501 aattatagtg cttaatattg ggaataagat taagcattat aattataatg tatgggcctg
1561 ttggtgtaag ctcagataat taaataaaaa tagcatgact caaatgagac atattctgct
1621 gaacagtttc tacttcctct cccgcctgtc ctgtcatggg agacgtgtat agttgctgct
1681 gtttcagcaa accaccataa gacgaaaatg cctcaggttg ggttgccagt cctttacaac
1741 tcagcttgaa tttcacaaca gtgattgtga gaatctgcgt ggtatacact gaaatatcgg
1801 tgtgctgtga tgcaaagctt acctttgacg atattgaatg tgatatagct gtagagaagt
1861 acttccttgc cttatgtgag gatttcaaac ttatttaaat tatgtagaca aatcaaagtg
1921 gcattgctta atttttagca ggcataataa gcaagttaac agtaaaatgc aaaacatgat
1981 aagcgttgct caatttttag caggtataat aagcaggtta acagtaaaaa tgcaaaacat
2041 gatagataag tcactttgaa aattcaaacc aaagttcctt caccttatgg aaataggaaa
2101 ttatggactt caaaattgga cacttcctgt ttacaaaaag aaattcagag ctaaaatcat
2161 ggtaaaaaaa aatagaaaca cttgagaact atggtcttta tgggtgcaat ttgaaatcct
2221 tttcatcatc ttaccagact aaactaagag cacataccaa acctatctta tggttgaaag
2281 ttggggttta ttttttatat gagaatatta tcactattac ataacatact caggacaaag
2341 aactttgctc agggaacata ccatgtaata tttttgttgt ttctttacag actagtctac
2401 agtcctgctt actcaaaaca aaccaaataa cttatacctt tatataagta ttatgtactg
2461 atgatagtaa ctacctctga gtttgacaca gatcaaaatt tttgaatatc agatatcagt
2521 tatcctattt ttatttcatg tgaaaactcc tctaaagcag attccctcaa ctctgtgcat
2581 atgtgaatat cactgatgtg aacacattgt tcatttacat aggtaaaata ttactctgtt
2641 tacagcaaaa ggctacctca tagttgatac atagcacacc tgtatgtatg ctgttccagc
2701 cttacaggtg gctgataatt ctctggtaca gaaccttttt atctgtatta taaatagcaa
2761 ttcacaactg catgtttctg acaaacactt gtgaataatg aagcatctcg ttttagttag
2821 caaagtctcc aaacatttcc ttaaaataat catgtattta gtttaaagaa ttatgggcac
2881 tgttcaactt aagcaaaaca gaacacggaa gcagtcttag aagcaccact ttgcccagag
2941 gtggaggttg gaaggggtag cagggagagg ggttggtgta tgcaggtatt catgctaggc
3001 aaagagttta aaagacgcca atgtccttca tttactgtct gtgctgccct gaagccaagc
3061 gtattgcagc attatagccc caggcacata actaactagc actggcttgc caaggaatga
3121 acatgcaatg ccattactag ctattgaggg aaaagggtct gtgtgaagca tcactttgca
3181 gggattacta atggtggggc agcaggtctg tgaattaagt tatctcttga cctcaccctc
3241 atgtcaacac aaatgtaatt cctaaacaag atgcattgcc agtctcttag ccctgtaagc
3301 tgatcttttg ctacatggca gactataatg aaaacatttt tatacttggg tttctagtct
3361 tcactagaag gccttggatg tatttttgca gttgaaagat ttagaaagat ttttacctgc
3421 ttataacttg gaagtttaga gtgcaatgta agaaaaaaga tcaagaaatg tcatgttatt
3481 agcatcagtc cacctccaat attgccgata ctttttttat tctggctcag ttttattttg
3541 caccagtgcg gccccaagtt actgctggtt gtatttagtt tgtgaatagg agcccataag
3601 tgttaataga ctttgtaaca ttcactataa gatgaattat acaggacatg ggaaatctca
3661 ttaagtctta aagttaattt aaattaattt atctgttttc tctaagaaat gtttatcata
3721 aaatatatat gtgtatttcc cctttggtta taaaatttgg gaaagtatgt acaagtgcag
3781 ctgcactgac tttaattttc tagatgtctt aatgagattt atttgtttta gagaagaaca
3841 tcttgttaaa agcatcaaac tctgtcttac atagctgtca acagcctctt taagatgtgg
3901 tggttgtatg atctgtgtct taattgttca gttagagtga gaagttgacc tatgattcat
3961 ttttaaattt tatatttgga acaaagctgc aagttatggt aaagtactgt actgtgagaa
4021 gtattatgat atttaatgca tctgtggctt aacacttgtg agagttacca gcttgaaaat
4081 gatggtgttg actacctctt gaatcacatc tatcaaccac tggcacctac caccaagctg
4141 gcttcaatta gtatgtgttg ctttttggta ttaacaacta accgtactag agaccaaagt
4201 gaaccctgat ttttatatgt ctttaataat ggtgttttat ctagtgtttt taaattatcc
4261 tgtgtagtat ttagattacc tcattgtcca ttttgactca tgttgtttac aagtgaaaat
4321 aaaaacactt gaactgtatg tttttaaaag acaaaaaagg ggtagatgtt tggaatgcgt
4381 ttcactcgca tgcagtcatc tggagggact gaagcactgt ttgcctttct gtacactctg
4441 ggttttatat tctcatttca tgcctaatgt cttattctgt caattatgga tatgttgagg
4501 tttaaaaaaa ttacttgatt aaaaataaaa catataacgt tggcatttaa aaaaaaaaaa
4561 a
```

TABLE IV
The sequence of the 3′ UTR of IGF2BPI showing the target seed sequences
to be deleted or modified in order to prevent binding with the LET-7
family of miRNAs as described herein. Target seed sequence positions
are listed as well as positions within in the entire
sequence of the 3′ UTR where they are shown in bold italics.

| miRNA Name | hsa-miR-4458 | | |
|---|---|---|---|
| Seed Location | 1632-1639, | | |
| | 1651-1657, | | |
| | 4269-4276, | | |
| | 4923-4930, | | |
| | 5568-5574 | | |
| NCBI Gene ID | 10642 | GenBank Accession | NM_001160423 |
| Gene Symbol | IGF2BP1 | 3′ UTR Length | 6701 |

| | |
|---|---|
| Gene Description | insulin like growth factor 2 mRNA binding protein 1 |

```
3′ UTR Sequence
      1 ccagcccctc cctgtccctt cgagtccagg acaacaacgg gcagaaatcg agagtgtgct
    61 ctccccggca ggcctgagaa tgagtgggaa tccgggacac ctgggccggg ctgtagatca
  121 ggtttgccca cttgattgag aaagatgttc cagtgaggaa ccctgatctc tcagccccaa
  181 acacccaccc aattggccca acactgtctg cccctcgggg tgtcagaaat tctagcgcaa
  241 ggcactttta aacgtggatt gtttaaagaa gctctccagg ccccaccaag agggtggatc
  301 acacctcagt gggaagaaaa ataaaatttc cttcaggttt taaaaacatg cagagaggtg
  361 ttttaatcag ccttaaagga tggttcattt cttgacctta atgtttttcc aatcttcttc
  421 cccctacttg ggtaattgat taaaatacct ccatttacgg cctctttcta tatttacact
  481 aattttttta tctttattgc taccagaaaa aaatgcgaac gaatgcattg ctttgcttac
  541 agtattgact caagggaaaa gaactgtcag tatctgtaga ttaattccaa tcactcccta
  601 accaataggt acaatacgga atgaagaaga ggggaaaatg gggagaaaga tggttaaaat
  661 acataataat ccacgtttaa aaggagcgca cttgtggctg atctatgcca gatcaccatc
  721 ttcaaattgg cacaactgaa atttccccac tctgttgggg cttccccacc acattcatgt
  781 ccctctcccg tgtaggtttc acattatgtc caggtgcaca taggtggtat tgaatgctca
  841 gcagggtagg ggctgaccac tgtccctgat tcccatcgtt ctcaggcgga ttttatattt
  901 ttttaaagtc tattttaatg attggatatg agcactggga aggggacgct aactcccctt
  961 gataaagtct cggttccatg gaggacttga gtggccccaa aggctgccac ggtgccctca
1021 ccccagccca tgtgctccca taagggctgg ttcctagagg caggggttgt ggggcactcc
1081 cagccacggc actgttacct tggtggtggg acttggaacc caaccctgag ctcccgataa
1141 agctaaagtc catcatctgg caaattcagt aaattggaga gtacttgctt ctgtttgtat
1201 ctgagaggaa tttttaactg acggcttctg tctccatgaa tcattatcag catgatgaaa
1261 ggtgtgtcta aaaaacaatt cagaatacca gcagcattgt acagcaaggg gtaaataagc
1321 ttaatttatt aatttaccag gcttaattaa gatcccatgg agtgtttagc ccttgtggga
1381 gacagaagcc atcagttaaa tgaggttagg cctctcctcc taatatactg attgacaatg
1441 catattagcc aggtaatgca ctttagctac cctggacaat gctatcaagt gtgctgggaa
1501 gggaggaagg cctctctaca tatggaaaag cccatgcgtg gagttcccct cctttcaaca
1561 ttgcaacaac agtaacaaca agacaaccgc aacatgtggg cgtagtcagg caatgctgtg
1621 tgcgaagtaa actacctcaa ggtatgaagt tacctcagca attattttcc tttttgttcc
1681 ccccaacccc attaaaaaaa tttttttttg atttttgttt ttttgcagct tgctgatatt
1741 ttatataaaa aagaaaagca aagcaaaaga gaagctgata gtcttgaata ttttattttt
1801 ttaatgaaaa gaaaaaacaa gaaagttatg tttcataatt tcttacaaca tgagccagta
1861 accctttagg aactctctat ggagaacagg cctggtggga aaggctttgg gggctgcccc
1921 cttaggagga ggctagtgct aagagggaag gcccaggttt gagagagccc agaggggcag
1981 agcccagagc cttgtttggc cctgatctct gacttctaga gccccagctg ctggcggctg
2041 ctggaatatc ctacctgata ggattaaaag gcctagtgga gctgggggct ctcagtggtt
2101 aaacaatgcc caacaaccaa ccagctggcc cttggtctcc tctctttcct cctttggtta
2161 aagagcatct cagccagctt ttcccaccag tggtgctgtt gagatatttt aaaatattgc
2221 ctccgtttta tcgaggagag aaataataac taaaaaatat accctttaaa aaaacctata
2281 tttctctgtc taaaaatatg ggagctgaga ttccgttcgt ggaaaaaaga caaggccacc
2341 ctctcgccct cagagaggtc cacctggttt gtcattgcaa tgcttttcat tttttttttt
2401 tgttattgtt tcatttcagt tccgtcttgc tattcttcct aatctatatc catagatcta
2461 aggggcaaac agatactagt taactgcccc cacctctgtc tccctgtctt ctttagatcg
2521 gtctgattga ttttaaaagt ggacccaaac ttagggaatt cttgatttag ggtggctggt
2581 ggcaaggagg ggcaggggat atggggacgt gactgggaca ggttcctgcc ttatcatttt
2641 ctccctagga cattcccttg tagcccccag aattgtctgg cccaaattga atagaagcag
2701 aaaaacattt agggataaca tcaggccagt agaattaagc ctctccacct gtcccaacca
2761 taaaaagggt ctcccagctt tccatctctg gctctatatg ctttatccca aaacaaagca
2821 gataacgttc agacgtcggc catttagtaa tttaaagcga atttccagca gcaagcatgc
2881 tttgatatct ggttcagact atcatcagga agaaaaaaaa atcccacagt acctgaaatg
2941 tgattgttgc agtgttcagt ttccttgggg gcctgctccc ttcacacctt gagcccaagt
3001 ccttttccgt tggctgattc agctcccaga agagacgagg aagtgtgtgg caagggactg
3061 gaaaacttca cttgcttgga ttaggcaagg ctccactcat tgttgatatt tgcccagcag
3121 gaaaatcatg taagttatac caccagaaag caaaaggagc atggtttggt ggttaaggtt
3181 tagtgggatg aaggacctgt cttggtgggc cgggccctct tgtgccccgt aggctaggtc
3241 ttagggcaac tccttgccct cctgctcagc acctccattt ccccatcctt ggtgagataa
3301 caagctatcg cgaaaagcac ttgggagatt tggatgattt gagaagagtg acttaaaaaa
3361 aatgcttctg tgctctaaga tatatatgtg tgtgtgtgtg ctacatatat atttttaaga
3421 aaggaccatc tctttaggat atatttttaa attctttgaa acacataacc aaaatggttt
3481 gattcactga ctgactttga agctgcatct gccagttaca ccccaaatgg ctttaatccc
3541 ctctcgggtc tggttgcctt ttgcagtttg ggttgtggac tcagctcctg tgaggggtct
3601 ggttaggaga gagccatttt taaggacagg gagttttata gcccttttct actttcctcc
3661 cctcctccca gtccttatca atcttttttc ctttttcctg accccctcct tctggaggca
3721 gttgggagct atccttgttt atgcctcact attggcagaa aagaccccat ttaaaaccca
3781 gagaacactg gagggggatg ctctagttgg ttctgtgtcc attttcctct gtgccaaaga
3841 cagacagaca gaggctgaga gaggctgttc ctgaatcaaa gcaatagcca gctttcgaca
3901 catacctggc tgtctgagga ggaaggcctc ctggaaactg ggagctaagg gcgaggccct
3961 tcccttcaga ggctcctggg ggattagggt gtggtgtttg ccaagccaag gggtagggag
4021 ccgagaaatt ggtctgtcgg ctcctggttg cactttgggg aaggagagga agtttggggc
4081 tccaggtagc tccctgttgt gggactgctc tgtcccctgc ccctactgca gagatagcac
4141 tgccgagttc ccttcaggcc tggcagacgg gcagtgagga ggggcctcag ttagctctca
4201 agggtgcctt cccctcctcc caacccagac ataccctctg ccaaactggg aaccagcagt
4261 gctagtaact acctcacaga gccccagagg gcctgcttga gccttcttgc tccacaggag
4321 aagctggtgc ctctaggcaa ccccttcctc ccacctctca tcaggggtgg gggttctcct
4381 ttctttcccc tgaagtgttt atggggagat cctagtggct ttgccattca aaccactcga
4441 ctgtttgcct gtttcttgaa aaccagtaga agggaaacag cacagcctgt cacagtaatt
4501 gcaggaagat tgaagaaaaa tcctcatcaa tgccagggga cataaaagcc atttcccttc
4561 caaatactcg acaatttaga tgcagaacat ttctctgtat tcagacttag agtaacacca
4621 gctgaaaact gcagtttctt tcctttggat acataaggct tctctatcgg ggtacgggac
4681 agggaggagg cctcatgtct gaagggggat ttaggggcga gagccccagc cctgaccctc
4741 ggtcctgtgc accgctttgg ggcacagtct gatggcgcct ttgctggcgc cttagtatgg
4801 ttgactccgg atggacaaaa gaaaaaaaat tttttttctt gaatgaaata gcaggaagct
4861 cctcgggagc atgtgttttg attaaccgca ggtgatggat gctacgagta taaatggatt
4921 aactacctca atccttacag taagattgga actaagggca gggactcatg cataagggta
4981 tgaatcccag ccaggacaag tgagttgagg cttgtgccac aaaaggtttg tccttgggga
5041 acaggcaggc ctgccaggat cccccccata tcgattgggc tgggagggct ggccatgagg
5101 tccccacttt ctgctttcct tgcccatgtg tcaccccttt ggcctccagc ttgtccctct
5161 ctcactttct atagctttgt tggaccagat ggtgaggaaa ggaatggcct cttcccttct
5221 agagggggct ggctggagtg agacctgggg cttggcctgg aacccaccac acagccccaa
5281 agtcaggaag cctggggaaa ccagagctga gacctcttca acagggtttc tttgagatcc
5341 tacacctcca ttgggccctt tttcagtctt caatgggggc ccagttggct ctagaaggag
5401 aagaggtgaa gcaggatcct ttgccctggg ggagtctgag ggcgcggtcc ttggactcat
5461 tcaggccgtc tttgtagttg ggggagttcc actgggcgat cccagcccct ccccacccac
5521 cctctaatgg acctcctcat agaagcccca tttcactttt gttttatcta cctcttagca
5581 aaacaataga taaattaggt agtggcagct ccacttgctt aggttagggg gggaaaaaga
5641 tttctttttc caaaggaaaa aaatattacc ttgagaatac tttccaaaaa ataaaattaa
5701 aaaaaaaaaa accaaaaaaa aaaatttttt tttaaaaggg agacattttc cagtgaccac
5761 tggattgttt taatttccca agcttttttt tcccccataa ataagtttca ctctttggcg
5821 attttcttca cttgtttaag ataacgtgct agctattcca acaggtaaca gctttcacag
5881 tctgcccctg gcctgtctca ccccatcccc caccctattc ctgccagtga gtccttcctg
5941 tgcttctctc ccttctcccc tcccagccag ctgacttcag tcacccctgt cccccctccc
6001 ctgccaataa gctcccccag gaataaaggc tttgttttgg ggatgcttaa atcttgactg
6061 gcacttcccg gctgtggggg ctggggagcc acttgtaaca tttctgtgca gattttatgt
6121 tagccactgc tatgtaaaag cacgttcaaa atgaatttca gcagattatg tgttaccata
6181 atgaataaac gtcctctatc accatttgga gtctcccttt tctccaggat cttgatcctg
6241 gtccccaaaa ccagagtgaa tcaaaagagc ttcctcccct gaggcaaagt ggatttgtaa
6301 gcagttctga aacatcactt actcagaaga gggaacgatg tattttgatg agtgcaaatt
6361 gggaagagct ggaggcctac tgcttgggac agtttttttt tttttttttt ttttaaatat
6421 gagtgctagc ttattctgta attgcggcaa ctttgaaaat tgtattttac tggaaatctg
6481 ccagccatca ccacccgatt ttgattgtat ccttcctccc atcctttaat ctgttcattg
6541 ctttggggga ggtggggcag ctggctcaca cgttggagtt tgttctttga tggatgaacg
6601 aacactccag ttttctttcc cgtgaaggtt gtttcagcca caaaccactt cattttgctg
6661 tttcaatttc aaaataaaag gaaacttata ttgaaagaca a
```

TABLE V
The sequence of the 3′ UTR of IGF2BP2 showing the target seed sequences
to be deleted or modified in order to prevent binding with the LET-7
family of miRNAs as described herein. Target seed sequence positions
are listed as well as positions within in the entire
sequence of the 3′ UTR where they are shown in bold italics.

| miRNA Name | hsa-let-7b-5p | | |
|---|---|---|---|
| Previous Name | hsa-let-7b | | |
| Seed Location | 1304-1311, | | |
| | 1320-1326, | | |
| | 2323-2329 | | |
| NCBI Gene ID | 10644 | GenBank Accession | NM_001291875 |
| Gene Symbol | IGF2BP2 | 3′ UTR Length | 2415 |

| | |
|---|---|
| Gene Description | insulin like growth factor 2 mRNA binding protein 2 |

```
3′ UTR Sequence
      1 ggctcccaca ggcaccagca aaacaacgga tgaatgtagc ccttccaaca cctgacagaa
    61 tgagaccaaa cgcagccagc cagatcggga gcaaaccaaa gaccatctga ggaatgagaa
  121 gtctgcggag gcggccaggg actctgccga ggccctgaga accccagggg ccgaggaggg
  181 gcggggaagg tcagccaggt ttgccagaac caccgagccc cgcctcccgc cccccagggc
  241 ttctgcaggc ttcagccatc cacttcacca tccactcgga tctctcctga actcccacga
  301 cgctatccct tttagttgaa ctaacatagg tgaacgtgtt caaagccaag caaaatgcac
  361 accctttttc tgtggcaaat cgtctctgta catgtgtgta catattagaa agggaagatg
  421 ttaagatatg tggcctgtgg gttacacagg gtgcctgcag cggtaatata ttttagaaat
  481 aatatatcaa ataactcaac taactccaat ttttaatcaa ttattaattt ttttttcttt
  541 ttaaagagaa agcaggcttt tctagacttt aaagaataaa gtctttggga ggtctcacgg
  601 tgtagagagg agctttgagg ccacccgcac aaaattcacc cagagggaaa tctcgtcgga
  661 aggacactca cggcagttct ggatcacctg tgtatgtcaa cagaagggat accgtctcct
  721 tgaagaggaa actctgtcac tcctcatgcc tgtctagctc atacacccat ttctctttgc
  781 ttcacaggtt ttaaactggt tttttgcata ctgctatata attctctgtc tctctctgtt
  841 tatctctccc ctccctcccc tccccttctt ctccatctcc attcttttga atttcctcat
  901 ccctccatct caatcccgta tctacgcacc cccccccccc caggcaaagc agtgctctga
  961 gtatcacatc acacaaaagg aacaaaagcg aaacacacaa accagcctca acttacactt
1021 ggttactcaa aagaacaaga gtcaatggta cttgtcctag cgttttggaa gaggaaaaca
1081 ggaacccacc aaaccaacca atcaaccaaa caaagaaaaa attccacaat gaaagaatgt
1141 attttgtctt tttgcatttt ggtgtataag ccatcaatat tcagcaaaat gattcctttc
1201 tttaaaaaaa aaaaatgtgg aggaaagtag aaatttacca aggttgttgg cccagggcgt
1261 taaattcaca gattttttta acgagaaaaa cacacagaag aagctacctc aggtgttttt
1321 acctcagcac cttgctcttg tgtttccctt agagattttg taaagctgat agttggagca
1381 tttttttatt tttttaataa aaatgagttg gaaaaaaaat aagatatcaa ctgccagcct
1441 ggagaaggtg acagtccaag tgtgcaacag ctgttctgaa ttgtcttccg ctagccaaga
1501 acctatatgg ccttcttttg gacaaacctt gaaaatgttt atttaaaaaa aaaaaagatg
1561 acaaagaaaa acagagagag agaatattgg agatgtcctg aattttaata gggtacgcgc
1621 cattagggct ttttgcgcta aaggatgaac atgtactggt ttatgtggac aagccattat
1681 accaccagac tgcaatgcca gtttcctcta ctgcaaacag tgttctgtga caaaaaaaaa
1741 aaaaaaaaaa agaaaaaaaa agaaaaaaca gaaatatatc cagctaacaa gatcctggtg
1801 tcttggtgtt ttattttact aggcattgcc tcatcccagg tgtcagctga gtcctagctc
1861 tgagggacct gggcagacag gcggtaggtg tgacctggat ccacacagct actctgagaa
1921 agctggtttt gcccaggaaa gtctttggga gaagctaaac tagatcggtg gtcaaagtgt
1981 agtccccaga ccagcagcat cagctatcat ctgggaactt gttagaaatg tagattcttg
2041 gggtccacct tgaatcagaa atcctggggg tgaggcccag caaccttttc agcaaggcct
2101 ctgggtgatt gctgtgcgca ctgaagtttc cgaacctcgg agctagaggg atggatcgac
2161 tgcatgtcaa gctagagaag accagtctat ccttaaggaa ctgcaagcct ggccaggcaa
2221 agctgctttt taagtggagg aaagggggag gataatatgc ccggcagttc cgggaagagg
2281 cacttcacgc ctccacggga agctcactag taagtgcagg catacctcag gaacgtcgca
2341 ggttcagttc cagaccacca cagtaaagcg aactttgcaa taaaatgagt catacacgtt
2401 aaaaaaaaaa aaaaa
```

TABLE VI
The sequence of the 3′ UTR of IGF2BP3 showing the target seed sequences
to be deleted or modified in order to prevent binding with the
LET-7 family of miRNAs as described herein. Target seed sequence
positions are listed as well as positions within in the entire
sequence of the 3′ UTR where they are shown in bold italics.

| miRNA Name | hsa-let-7a-5p | | |
|---|---|---|---|
| Previous Name | hsa-let-7a | | |
| Seed Location | 560-567 | | |
| NCBI Gene ID | 10643 | GenBank Accession | NM_006547 |
| Gene Symbol | IGF2BP3 | 3′ UTR Length | 2162 |

| | |
|---|---|
| Gene Description | insulin like growth factor 2 mRNA binding protein 3 |

```
3′ UTR Sequence
      1 aggctcagga aacagcccac cacagaggca gatgccaaac caaagacaga ttgcttaacc
    61 aacagatggg cgctgacccc ctatccagaa tcacatgcac aagtttttac ctagccagtt
  121 gtttctgagg accaggcaac ttttgaactc ctgtctctgt gagaatgtat actttatgct
  181 ctctgaaatg tatgacaccc agctttaaaa caaacaaaca aacaaacaaa aaaagggtgg
  241 gggagggagg gaaagagaag agctctgcac ttccctttgt tgtagtctca cagtataaca
  301 gatattctaa ttcttcttaa tattccccca taatgccaga aattggctta atgatgcttt
  361 cactaaattc atcaaataga ttgctcctaa atccaattgt taaaattgga tcagaataat
  421 tatcacagga acttaaatgt taagccatta gcatagaaaa actgttctca gttttatttt
  481 tacctaacac taacatgagt aacctaaggg aagtgctgaa tggtgttggc aggggtatta
  541 aacgtgcatt tttactcaac tacctcaggt attcagtaat acaatgaaaa gcaaaattgt
  601 tccttttttt tgaaaatttt atatacttta taatgataga agtccaaccg ttttttaaaa
  661 aataaattta aaatttaaca gcaatcagct aacaggcaaa ttaagatttt tacttctggc
  721 tggtgacagt aaagctggaa aattaatttc agggtttttt gaggcttttg acacagttat
  781 tagttaaatc aaatgttcaa aaatacggag cagtgcctag tatctggaga gcagcactac
  841 catttattct ttcatttata gttgggaaag tttttgacgg tactaacaaa gtggtcgcag
  901 gagattttgg aacggctggt ttaaatggct tcaggagact tcagtttttt gtttagctac
  961 atgattgaat gcataataaa tgctttgtgc ttctgactat caatacctaa agaaagtgca
1021 tcagtgaaga gatgcaagac tttcaactga ctggcaaaaa gcaagcttta gcttgtctta
1081 taggatgctt agtttgccac tacacttcag accaatggga cagtcataga tggtgtgaca
1141 gtgtttaaac gcaacaaaag gctacatttc catggggcca gcactgtcat gagcctcact
1201 aagctatttt gaagattttt aagcactgat aaattaaaaa aaaaaaatta gactccacct
1261 taagtagtaa agtataacag gatttctgta tactgtgcaa tcagttcttt gaaaaaaaag
1321 tcaaaagata gagaatacaa gaaaagtttt tgggatataa tttgaatgac tgtgaaaaca
1381 tatgaccttt gataacgaac tcatttgctc actccttgac agcaaagccc agtacgtaca
1441 attgtgttgg gtgtgggtgg tctccaaggc cacgctgctc tctgaattga ttttttgagt
1501 tttgtttgta agatgatcac agtcatgtta cactgatcta aaggacatat atataaccct
1561 ttaaaaaaaa aatcactgcc tcattcttat ttcaagatga atttctatac agactagatg
1621 tttttctgaa gatcaattag acattttgaa aatgatttaa agtgttttcc ttaatgttct
1681 ctgaaaacaa gtttcttttg tagttttaac caaaaaagtg ccctttttgt cactggattc
1741 tcctagcatt catgattttt ttttcataca atgaattaaa attgctaaaa tcatggactg
1801 gctttctggt tggatttcag gtaagatgtg tttaaggcca gagcttttct cagtatttga
1861 tttttttccc caatatttga ttttttaaaa atatacacat aggtgctgca tttatatctg
1921 ctggtttaaa ttctgtcata tttcacttct agccttttag tatggcaaat catattttac
1981 ttttacttaa gcatttgtaa tttggagtat ctggtactag ctaagaaata attctataat
2041 tgagttttgt actcaccata tatggatcat tcctcatgta taatgtgccc caaatgcagc
2101 ttcattttcc agataccttg acgcagaata aattttttca tcatttaggt gcaaaaaaaa
2161 aa
```

TABLE VII
The sequence of the 3′ UTR of HMGA2 showing the target seed sequences
to be deleted or modified in order to prevent binding with the
LET-7 family of miRNAs as described herein. Target seed sequence
positions are listed as well as positions within in the entire
sequence of the 3′ UTR where they are shown in bold italics.

| miRNA Name | hsa-let-7a-5p | | |
|---|---|---|---|
| Previous Name | hsa-let-7a | | |
| Seed Location | 21-28, | Seed Location | 21-28, |
| | 1107-1113, | | 1107-1113, |
| | 1256-1262, | | 1256-1262, |
| | 1619-1625, | | 1619-1625, |
| | 1668-1674, | | 1668-1674, |
| | 2521-2527, | | 2521-2527, |
| | 2541-2547 | | 2541-2547 |
| NCBI Gene ID | 8091 | GenBank Accession | NM_003483 |
| Gene Symbol | HMGA2 | 3′ UTR Length | 3009 |

| | |
|---|---|
| Gene Description | high mobility group AT-hook 2 |

```
      1 ggggcgccaa cgttcgattt ctacctcagc agcagttgga tcttttgaag ggagaagaca
    61 ctgcagtgac cacttattct gtattgccat ggtctttcca ctttcatctg gggtggggtg
  121 gggtggggtg ggggaggggg gggtggggtg gggagaaatc acataacctt aaaaaggact
  181 atattaatca ccttctttgt aatcccttca cagtcccagg tttagtgaaa aactgctgta
  241 aacacagggg acacagctta acaatgcaac ttttaattac tgttttcttt tttcttaacc
  301 tactaatagt ttgttgatct gataagcaag agtgggcggg tgagaaaaac cgaattgggt
  361 ttagtcaatc actgcactgc atgcaaacaa gaaacgtgtc acacttgtga cgtcgggcat
  421 tcatatagga agaacgcggt gtgtaacact gtgtacacct caaataccac cccaacccac
  481 tccctgtagt gaatcctctg tttagaacac caaagataag gactagatac tactttctct
  541 ttttcgtata atcttgtaga cacttacttg atgattttta actttttatt tctaaatgag
  601 acgaaatgct gatgtatcct ttcattcagc taacaaacta gaaaaggtta tgttcatttt
  661 tcaaaaaggg aagtaagcaa acaaatattg ccaactcttc tatttatgga tatcacacat
  721 atcagcagga gtaataaatt tactcacagc acttgttttc aggacaacac ttcattttca
  781 ggaaatctac ttcctacaga gccaaaatgc catttagcaa taaataacac ttgtcagcct
  841 cagagcattt aaggaaacta gacaagtaaa attatcctct ttgtaattta atgaaaaggt
  901 acaacagaat aatgcatgat gaactcacct aattatgagg tgggaggagc gaaatctaaa
  961 tttcttttgc tatagttata catcaattta aaaagcaaaa aaaaaaaagg ggggggcaat
1021 ctctctctgt gtctttctct ctctctcttc ctctccctct ctcttttcat tgtgtatcag
1081 tttccatgaa agacctgaat accacttacc tcdaattaag catatgtgtt acttcaagta
1141 atacgttttg acataagatg gttgaccaag gtgcttttct tcggcttgag ttcaccatct
1201 cttcattcaa actgcacttt tagccagaga tgcaatatat ccccactact caatactacc
1261 tctgaatgtt acaacgaatt tacagtctag tacttattac atgctgctat acacaagcaa
1321 tgcaagaaaa aaacttactg ggtaggtgat tctaatcatc tgcagttctt tttgtacact
1381 taattacagt taaagaagca atctccttac tgtgtttcag catgactatg tatttttcta
1441 tgttttttta attaaaaatt tttaaaatac ttgtttcagc ttctctgcta gatttctaca
1501 ttaacttgaa aattttttaa ccaagtcgct cctaggttct taaggataat tttcctcaat
1561 cacactacac atcacacaag atttgactgt aatatttaaa tattaccctc caagtctgta
1621 cctcdaatga attctttaag gagatggact aattgacttg caaagaccta cctccagact
1681 tcaaaaggaa tgaacttgtt acttgcagca ttcatttgtt ttttcaatgt ttgaaatagt
1741 tcaaactgca gctaacccta gtcaaaacta tttttgtaaa agacatttga tagaaaggaa
1801 cacgttttta catacttttg caaaataagt aaataataaa taaaataaaa gccaaccttc
1861 aaagaaactt gaagctttgt aggtgagatg caacaagccc tgcttttgca taatgcaatc
1921 aaaaatatgt gtttttaaga ttagttgaat ataagaaaat gcttgacaaa tattttcatg
1981 tattttacac aaatgtgatt tttgtaatat gtctcaacca gatttatttt aaacgcttct
2041 tatgtagagt ttttatgcct ttctctccta gtgagtgtgc tgacttttta acatggtatt
2101 atcaactggg ccaggaggta gtttctcatg acggcttttg tcagtatggc ttttagtact
2161 gaagccaaat gaaactcaaa accatctctc ttccagctgc ttcagggagg tagtttcaaa
2221 ggccacatac ctctctgaga ctggcagatc gctcactgtt gtgaatcacc aaaggagcta
2281 tggagagaat taaaactcaa cattactgtt aactgtgcgt taaataagca aataaacagt
2341 ggctcataaa aataaaagtc gcattccata tctttggatg ggccttttag aaacctcatt
2401 ggccagctca taaaatggaa gcaattgctc atgttggcca aacatggtgc accgagtgat
2461 ttccatctct ggtaaagtta cacttttatt tcctgtatgt tgtacaatca aaacacacta
2521 ctacctctta agtcccagta tacctcattt ttcatactga aaaaaaaagc ttgtggccaa
2581 tggaacagta agaacatcat aaaattttta tatatatagt ttatttttgt gggagataaa
2641 ttttatagga ctgttctttg ctgttgttgg tcgcagctac ataagactgg acatttaact
2701 tttctaccat ttctgcaagt taggtatgtt tgcaggagaa aagtatcaag acgtttaact
2761 gcagttgact ttctccctgt tcctttgagt gtcttctaac tttattcttt gttctttatg
2821 tagaattgct gtctatgatt gtactttgaa tcgcttgctt gttgaaaata tttctctagt
2881 gtattatcac tgtctgttct gcacaataaa cataacagcc tctgtgatcc ccatgtgttt
2941 tgattcctgc tctttgttac agttccatta aatgagtaat aaagtttggt caaaacagaa
3001 aaaaaaaaa
```

### DETAILED DESCRIPTION

### Abbreviations

3′-UTR—The 3′ untranslated region of an mRNA transcript
5′-UTR—The 5′ untranslated region of an mRNA transcript
AC—Adult-derived cells
AMH—Anti-Mullerian Hormone
ASC—Adult stem cells
CGMP—Current Good Manufacturing Processes
CM—Cancer Maturation
CNS—Central Nervous System
DMEM—Dulbecco's modified Eagle's medium
DMSO—Dimethyl sulphoxide
DNAm—Changes in the methylation of DNA that provide a marker or “clock” of the age of cells and tissue.
DPBS—Dulbecco's Phosphate Buffered Saline
DR-iTR—Developmentally-Regulated induced Tissue Regeneration
DR-O—Developmentally-Regulated Oncolysis
ED Cells—Embryo-derived cells; hED cells are human ED cells
EDTA—Ethylenediamine tetraacetic acid
EFT—Embryonic-Fetal Transition and refers to the transition occurring at approximately eight weeks of human gestational development where organogenesis is essentially complete and many cells and tissues such as skin lose the capacity for scarless epimorphic regeneration.
EG Cells—Embryonic germ cells; hEG cells are human EG cells
EP—Embryonic progenitor cells (cells from embryonic anlagen such as PSC-derived cells that while potentially differentiated or potentially multipotent, nevertheless have not matured to a non-regenerative state.
ES Cells—Embryonic stem cells; hES cells are human ES cells
ESC—Embryonic Stem Cells
EVs—Extracellular vesicles including exosomes and other vesicles released by cells.
FACS—Fluorescence activated cell sorting
FBS—Fetal bovine serum
FPKM—Fragments Per Kilobase of transcript per Million mapped reads from RNA sequencing.
GFER—Growth Factor, Augmenter of Liver Regeneration (ALR)
GFP—Green fluorescent protein
GMP—Good Manufacturing Practices
HAEC—Human Aortic Endothelial Cell
hED Cells—Human embryo-derived cells
hEG Cells—Human embryonic germ cells are stem cells derived from the primordial germ cells of fetal tissue.
hEP—Human embryonic progenitor cells
hESC—Human Embryonic Stem Cells
HGPS—Hutchinson-Gilford Progeria Syndrom (progeria)
hiPS—Cells Human induced pluripotent stem cells are cells with properties similar to hES cells obtained from somatic cells after exposure to hES-specific transcription factors such as SOX2, KLF4, OCT4, MYC, or NANOG, LIN28, OCT4, and SOX2.
HSE—Human skin equivalents are mixtures of cells and biological or synthetic matrices manufactured for testing purposes or for therapeutic application in promoting wound repair.
iCM—Induced Cancer Maturation.
iPS Cells—Induced pluripotent stem cells are cells with properties similar to hES cells obtained from somatic cells after exposure to ES-specific transcription factors such as SOX2, KLF4, OCT4, MYC, or NANOG, LIN28, OCT4, and SOX2, SOX2, KLF4, OCT4, MYC, and (LIN28A or LIN28B), or other combinations of OCT4, SOX2, KLF4, NANOG, ESRRB, NR5A2, CEBPA, MYC, LIN28A and LIN28B.
IRES—Internal Ribosome Entry Site
iS-CSC—induced Senolysis of Cancer Stem Cells refers to the treatment of cells in malignant tumors that are refractory to ablation by chemotherapeutic agents or radiation therapy wherein said iS-CSC treatment causes said refractory cells to revert to a pre-fetal pattern of gene expression and become sensitive to chemotherapeutic agents or radiation therapy.
iTM—Induced Tissue Maturation
iTR—Induced Tissue Regeneration
LET-7—LET-7 is an inclusive term for all members of the LET-7 family of miRNAs defined herein as canonical genes and RNA transcripts in varied stages of processing to mature miRNA as well as MIR-98, MIR-4458, and MIR-4500 that share the same seed sequence as the canonical LET-7 genes. Therefore, the “LET-7 Family” includes the genes and processed transcripts listed in Table I.
LNP—Lipid nanoparticle
MEM—Minimal essential medium
MSC—Mesenchymal stem cell
NT—Neonatal Transition which is characterized by alterations in gene expression occurring at or around the time of normal birth. Examples of genes that function as markers of NT are imprinted genes such as IGF2 and MEST, both of which are markedly decreased in expression beginning at approximately one week post-natal.
PBS—Phosphate buffered saline
PPT—Prenatal-Postnatal Transition refers to the molecular alterations that occur in cells of placental mammals at or within a week of birth.
PS fibroblasts—Pre-scarring fibroblasts are fibroblasts derived from the skin of early gestational skin or derived from ED cells that display a prenatal pattern of gene expression in that they promote the rapid healing of dermal wounds without scar formation.
PT—Pluripotency Transition which coincides in vivo with the exit from the germ-line and entry into the three germ layers or extraembryonic cell lineages but not germ line cells such as primordial germ cells or embryonic germ cells. In vitro, the PT would represent the cells derived from hPSCs that have entered the process of differentiation as evidenced by the repression of pluripotency markers such as expression from the genes OCT4, SOX2, and TERT.
RFU—Relative Fluorescence Units
RNA-seq—RNA sequencing
SFM—Serum-Free Medium
ST—Senescence Transition refers to the transition of a cell capable of proliferating given normal growth stimulation signals to a cell that has undergone a checkpoint arrest characterized by elevated expression of cell cycle inhibitory genes such as CDKN2A, and often a result of telomere shortening.
St. Dev.—Standard Deviation
TR—Tissue Regeneration
Vg—Viral genomes

### Definitions

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. application Nos. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application no. 60/8188 13, filed Jul. 5, 2006, PCT/US06/30632, filed Aug. 3, 2006, the disclosure of each of which is incorporated by reference herein).

The term “blastomere/morula cells” refers to blastomere or morula cells in a mammalian embryo or blastomere or morula cells cultured in vitro with or without additional cells including differentiated derivatives of those cells.

The term “cell line” refers to a mortal or immortal population of cells that is capable of propagation and expansion in vitro.

The term “cells, tissues, and organs” refers to corresponding discrete components of an animal's body containing at minimum one, preferably a million, and most preferably a billion viable cells despite residing within a postmortem body and despite having been subjected to disease, trauma, surgery, ischemia, bums, or other causes of cell damage or death. Said cells, tissues, and organs include all somatic cells, tissues, and organs of that animal including but not limited to: limb, digit, cartilage, heart, blood vessel, bone, esophagus, stomach, liver, gallbladder, pancreas, intestines, rectum, anus, endocrine gland (e.g., thyroid, parathyroid, adrenal, endocrine portion of pancreas), skin, hair follicle, thymus, spleen, skeletal muscle, focal damaged cardiac muscle, smooth muscle, brain, spinal cord, peripheral nerve, ovary, fallopian tube, uterus, vagina, mammary gland, testes, vas deferens, seminal vesicle, prostate, penis, pharynx, larynx, vocal cords, trachea, bronchi, lungs, kidney, ureter, bladder, urethra, eye (including components of the eye such as the retina or cornea), and the ear (including components of the middle and inner ear such as the organ of Corti).

The term “cytoplasmic bleb” refers to the cytoplasm of a cell bound by an intact or permeabilized but otherwise intact plasma membrane, but lacking a nucleus. The term “differentiated cells” when used in reference to cells made by methods of this disclosure from pluripotent stem cells refer to cells having reduced potential to differentiate when compared to the parent pluripotent stem cells. The differentiated cells of this disclosure comprise cells that could differentiate further (i.e., they may not be terminally differentiated). The term “embryonic” or “embryonic stages of development” refers to prenatal stages of development of cells, tissues or animals, specifically, the embryonic phases of development of cells compared to fetal and adult cells. In the case of the human species, the transition from embryonic to fetal development occurs at about 8 weeks of prenatal development, in mouse it occurs on or about 16 days, and in the rat species, at approximately 17.5 days post coitum. The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species).

The term “decellularization” as used in the present invention refers to the removal of cells from a tissue in vivo while leaving the surrounding ECM of that tissue essentially intact. Said removal of cells may be achieved by any means including by means of nonlimiting example, the lysis of said cells in a tissue by means of a detergent.

The term “differentiation” as used in the present invention refers to change of phenotype of cells from a state of pluripotency or multipotency to a state of reduced or absent multipotency wherein the cells take on individual characteristics of and reach their fetal or adult form and function. Cells may differentiate in vivo without maturing as in the case of animals capable of epimorphic regeneration. In the case of cells in vitro, pluripotent stem cells may differentiate in recognizable cell types such as vascular endothelial cells or cardiomyocytes while not maturing as evidenced by their continuing to express RNA markers of immature cells such as isoforms of the alpha and beta clustered protocadherin locus such as PCDHB2 and lack expression of fetal and adult markers such as COX7A1 and PCDHGA12.

The term “extracellular matrix” as utilized herein, refers to the assembly of proteins, proteoglycans, loosely associated signaling molecules, and other molecules in which cells of a given tissue normally reside.

The term “global activator of TR” or “global activator of iTR” refers to agents including combinations of the expressed genes OCT4, SOX2, KLF4, NANOG, ESRRB, NR5A2, CEBPA, MYC, LIN28A, TERT, and LIN28B, or their corresponding mRNAs and proteins, and including genetically-modified versions of aforementioned genes including but not limited to modified SOX2 as described (Highly cooperative chimeric super-SOX induces naive pluripotency across species, Maccarthy, Caitlin M. et al. Cell Stem Cell, Volume 31, Issue 1, 127-147.e9); capable of modulating in cells, tissues, or organs of a metazoan animal such as a mammal or a human a multiplicity of iTR marker genes including, but not limited to, agents capable of downregulating COX7A1 expression while simultaneously up-regulating expression of PCDHB2, or downregulating expression of NAALADL1 while simultaneously up-regulating expression of AMH in cells derived from fetal or adult sources and are capable of inducing a pattern of gene expression leading to increased scarless tissue regeneration in response to tissue damage or degenerative disease but without reprogramming cells in said cells, tissues, or organs of a metazoan animal such as a mammal or a human to pluripotency. The term “human embryonic stem cells” (hES cells) refers to human ES cells. The term “human induced pluripotent stem cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC; OCT4 or SOX2, OCT4, NANOG, and LIN28; or various combinations of OCT4, SOX2, KLF4, NANOG, ESRRB, NR5A2, CEBPA, MYC, LIN28A and LIN28B, their corresponding mRNAs or proteins, and including genetically-modified versions of aforementioned genes including but not limited to modified SOX2 as described (Highly cooperative chimeric super-SOX induces naive pluripotency across species, Maccarthy, Caitlin M. et al. Cell Stem Cell, Volume 31, Issue 1, 127-147.e9); or other methods that induce somatic cells to attain a pluripotent stem cell state with properties similar to hES cells. However, the reprogramming of somatic cells by somatic cell nuclear transfer (SCNT) are typically referred to as NT-ES cells as opposed to iPS cells.

The term “induced pre-regenerative tissue” refers to cells, tissues, or organs of a metazoan that have been exposed to iTR factors such that the cells including those residing in tissues or organs express a pattern of gene expression normally characteristic of a prenatal state, more preferably of a pre-EFT state, but where said cells, tissues, and organs have not yet completed the course of regeneration to restore the anatomical or physiological state characteristic of a normal tissue or organ for that organism at its point in its development.

The term “induced tissue regeneration” or “iTR” refers to the use of the methods of the present disclosure to alter the molecular composition of fetal or adult mammalian cells such that said cells have reversed developmental aging and are capable or regenerating functional tissue following damage to that tissue wherein said regeneration would not be the normal outcome of said fetal or adult cells if untreated by iTR in animals of that species or when said normal untreated cells are introduced into decellularized tissue in a postmortem animal as described herein.

The term “isolated” refers to a substance that is (i) separated from at least some other substances with which it is normally found in nature, usually by a process involving the hand of man, (ii) artificially produced (e.g., chemically synthesized), and/or (iii) present in an artificial environment or context (i.e., an environment or context in which it is not normally found in nature).

The term “iTR activators” or “TR activators” refers to genes, RNAs, or proteins whose expression in embryonic phases of development facilitate TR. iTR activators as described herein include the subsets of Global iTR activators and Segmental iTR activators as described herein as Description of iTR Factors.

The term “iTR factors” refers to molecules that alter the levels of TR activators and TR inhibitors in a manner leading to TR in a tissue not naturally capable of TR

The term “iTR genes” refers to the subset of iTR activators that are genes that when altered in expression or introduced into tissues as RNA can cause induced tissue regeneration in tissues not normally capable of such regeneration. Nonlimiting examples of iTR factors include gene therapy vectors expressing transiently or constitutively the global iTR activator and segmental activator genes listed in herein under the heading of Description of iTR Factors or extracellular vesicles designed to deliver mRNA or DNA encoding the sequences also detailed herein under the heading of Description of iTR Factors to a postmortem animal.

The term “life support technologies” refers to artificial methods of maintaining viability of the majority of cells on the body of a metazoan animal including but not limited to hypothermia, mechanical ventilation, extracorporeal membrane oxygenation (ECMO), dialysis, cardiac assist devices, or the transient use of artificial organs wherein said viability would otherwise not occur.

The term “maturation” refers to the change in phenotype of somatic cells such that said cells no longer are capable of scarless (epimorphic) regeneration. Cells may differentiate in vivo without maturation as in the case of animals capable of epimorphic regeneration. In the case of cells in vitro, pluripotent stem cells may differentiate in recognizable cell types such as vascular endothelial cells or cardiomyocytes while not maturing as evidenced by their continuing to express RNA markers of immature pre-EFT cells such as isoforms of the alpha and beta clustered protocadherin locus such as PCDHB2 and lack expression of fetal and adult markers such as COX7A1 and PCDHGA12.

The term “metazoan” refers to any species in the Kingdom of Animalia (also known as Metazoa) that comprises all animals having a body composed of multiple differentiated cells that include at minimum a digestive cavity lined with specialized differentiated cells.

The term “nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments, a nucleic acid comprises standard nucleosides (abbreviated A, G, C, T, U). In other embodiments, a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos. In some embodiments, a nucleic acid comprises nucleosides that are linked by phosphodiester bonds, as in DNA and RNA In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, or antisense-based molecules for research or therapeutic purposes are contemplated for use in various embodiments of the instant disclosure. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., in vivo, relative to RNA or DNA of the same length and strandedness. In some embodiments, a modification decreases immunogenicity of a nucleic acid relative to RNA or DNA of the same length and strandedness. In some embodiments, between 5% and 95% of the nucleosides in one or both strands of a nucleic acid are modified. Modifications may be located uniformly or nonuniformly, and the location of the modifications (e.g., near the middle, near or at the ends, alternating, etc.) can be selected to enhance desired propert(ies). A nucleic acid may comprise a detectable label, e.g., a fluorescent dye, radioactive atom, etc. “Oligonucleotide” refers to a relatively short nucleic acid, e.g., typically between about 4 and about 60 nucleotides long. Where reference is made herein to a polynucleotide, it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity such as a carbohydrate group, a phosphate group, a fatty acid group, a linker for conjugation, functionalization, etc.

A polypeptide that has a nonpolypeptide moiety covalently or noncovalently associated therewith is still considered a “polypeptide”. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide.

A polypeptide sequence presented herein is presented in an N-terminal to C-terminal direction unless otherwise indicated. A polypeptide may be cyclic or contain a cyclic portion. Where a naturally occurring polypeptide is discussed herein, it will be understood that the disclosure encompasses embodiments that relate to any isoform thereof (e.g., different proteins arising from the same gene as a result of alternative splicing or editing of mRNA or as a result of different alleles of a gene, e.g., alleles differing by one or more single nucleotide polymorphisms (typically such alleles will be at least 95%, 96%, 97%, 98%, 99%, or more identical to a reference or consensus sequence). A polypeptide may comprise a sequence that targets it for secretion or to a particular intracellular compartment (e.g., the nucleus) and/or a sequence targets the polypeptide for post-translational modification or degradation. Certain polypeptides may be synthesized as a precursor that undergoes post-translational cleavage or other processing to become a mature polypeptide. In some instances, such cleavage may only occur upon particular activating events. Where relevant, the disclosure provides embodiments relating to precursor polypeptides and embodiments relating to mature versions of a polypeptide lifespan.

The term “postmortem” refers to state of a metazoan animal including human that has died as determined by the inability to be resuscitated by natural means. In the case of mammals, the term can, for instance, be applied to an animal that has ceased cardiac activity or respiration and will not reinitiate said cardiac or respiratory activity without artificial life support. Alternatively, in the case of humans, the term may, for example, refer to an individual with no discernable brain wave activity detectable by an electroencephalogram on two 24 hour consecutive measurements. Notwithstanding the above, said postmortem state includes metazoans including humans that have cells, tissues, and organs in the body that may be viable in the sense that they are useful in transplantation therapy or may be in a state of deterioration such that they are marginally or not useful in transplantation therapy without the induction of tissue regeneration as taught herein.

The term “prenatal” refers to a stage of embryonic development of a placental mammal prior to which an animal is not capable of viability apart from the uterus.

The term “primordial stem cells” refers collectively to pluripotent stem cells capable of differentiating into cells of all three primary germ layers: endoderm, mesoderm, and ectoderm, as well as neural crest. Therefore, examples of primordial stem cells would include but not be limited by human or non-human mammalian ES cells or cell lines, blastomere/morula cells and their derived ED cells, iPS, and EG cells.

The term “purified” refers to agents or entities (e.g., compounds) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure.

In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

The term “regenerative recellularization” as described in the instant application refers to the introduction of living cells into a decellularized tissue wherein said cells are preferably in a quantity and phenotype capable of repopulating said decellularized tissue and most preferably are cells that have been treated so as to reprogram the cells to a pattern of gene expression such that the cells have increased potential for tissue regeneration, said reprogramming most preferably is by means of iTR as described herein.

The term “RNA interference” (RNAi) is used herein consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In certain embodiments a first strand of the dsRNA is at least 80%, 85%, 90%, 95%, or 100% complementary to a target mRNA and the other strand of the dsRNA is at least 80%, 85%, 90%, 95%, or 100% complementary to the first strand. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. siRNAs are typically double-stranded oligonucleotides having 16-30, e.g., 16, 17, 18, 19, 20, 21, 22, 23, 24, 25, 26, 27, 28, 29, or 30 nucleotides (nt) in each strand, wherein the double-stranded oligonucleotide comprises a double-stranded portion between 15 and 29 nucleotides long and either or both of the strands may comprise a 3′ overhang between, e.g., 1-5 nucleotides long, or either or both ends can be blunt. In some embodiments, an siRNA comprises strands between 19 and 25 nt, e.g., between 2 1 and 23 nucleotides long, wherein one or both strands comprises a 3′ overhang of 1-2 nucleotides. One strand of the double-stranded portion of the siRNA (termed the “guide strand” or “antisense strand”) is substantially complementary (e.g., at least 80% or more, e.g., 85%, 90%, 95%, or 100%) complementary to (e.g., having 3, 2, 1, or 0 mismatched nucleotide(s)) a target region in the mRNA, and the other double-stranded portion is substantially complementary to the first double-stranded portion. In many embodiments, the guide strand is 100% complementary to a target region in an mRNA and the other passenger strand is 100% complementary to the first double-stranded portion (it is understood that, in various embodiments, the 3′ overhang portion of the guide strand, if present, may or may not be complementary to the mRNA when the guide strand is hybridized to the mRNA). In some embodiments, a shRNA molecule is a nucleic acid molecule comprising a stem-loop, wherein the double-stranded stem is 16-30 nucleotides long and the loop is about 1-10 nucleotides long. siRNA can comprise a wide variety of modified nucleosides, nucleoside analogs and can comprise chemically or biologically modified bases, modified backbones, etc. Without limitation, any modification recognized in the art as being useful for RNAi can be used. Some modifications result in increased stability, cell uptake, potency, etc. Some modifications result in decreased immunogenicity or clearance. In certain embodiments the siRNA comprises a duplex about 19-23 (e.g., 19, 20, 21, 22, or 23) nucleotides in length and, optionally, one or two 3′ overhangs of 1-5 nucleotides in length, which may be composed of deoxyribonucleotides. shRNA comprise a single nucleic acid strand that contains two complementary portions separated by a predominantly non-self complementary region. The complementary portions hybridize to form a duplex structure and the non-self complementary region forms a loop connecting the 3′ end of one strand of the duplex and the 5′ end of the other strand. shRNAs undergo intracellular processing to generate siRNAs. Typically, the loop is between 1 and 8, e.g., 2-6 nucleotides long. MicroRNAs (miRNAs) are small, naturally occurring, non-coding, single-stranded RNAs of about 21-25 nucleotides (in mammalian systems) that inhibit gene expression in a sequence-specific manner. They are generated intracellularly from precursors (pre-miRNA) having a characteristic secondary structure comprised of a short hairpin (about 70 nucleotides in length) containing a duplex that often includes one or more regions of imperfect complementarity which is in turn generated from a larger precursor (pri-miRNA). Naturally occurring miRNAs are typically only partially complementary to their target mRNA and often act via translational repression. RNAi agents modelled on endogenous miRNA or miRNA precursors are of use in certain embodiments of the disclosure. For example, an siRNA can be designed so that one strand hybridizes to a target mRNA with one or more mismatches or bulges mimicking the duplex formed by a miRNA and its target mRNA.

Such siRNA may be referred to as miRNA mimics or miRNA-like molecules. miRNA mimics may be encoded by precursor nucleic acids whose structure mimics that of naturally occurring miRNA precursors. In certain embodiments an RNAi agent is a vector (e.g., a plasmid or virus) that comprises a template for transcription of an siRNA (e.g., as two separate strands that can hybridize to each other), shRNA, or microRNA precursor.

Typically the template encoding the siRNA, shRNA, or miRNA precursor is operably linked to expression control sequences (e.g., a promoter), as known in the art. Such vectors can be used to introduce the template into vertebrate cells, e.g., mammalian cells, and result in transient or stable expression of the siRNA, shRNA, or miRNA precursor. Precursors (shRNA or miRNA precursors) are processed intracellularly to generate siRNA or miRNA.

In general, small RNAi agents such as siRNA can be chemically synthesized or can be transcribed in vitro or in vivo from a DNA template either as two separate strands that then hybridize, or as an shRNA which is then processed to generate an siRNA. Often RNAi agents, especially those comprising modifications, are chemically synthesized. Chemical synthesis methods for oligonucleotides are well known in the art.

The term “segmental reprogramming factor” refers to molecules that are capable of inducing a regenerative state by not capable of reverting cells and/or tissue to a state of pluripotency. Said segmental reprogramming factors include: IGF2BP1, IGF2BP2, IGF2BP3, LIN28A, HMGA2, H19, LIN28B, TERT, WTAP, METTL3, METTL14, and the metabolite L-2-HG, RNAi directed to the eraser FTO, and/or molecules inactivating IGFBP6 such as monoclonal antibody-based competitive inhibition, or a combination of the aforementioned factors.

The term “small molecule” as used herein, is an organic molecule that is less than about 2 kilodaltons (KDa) in mass. In some embodiments, the small molecule is less than about 1.5 kDa, or less than about 1.0 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

The term “small molecule iTR activators” are small molecules applied singly or in combination with other small molecule iTR activators or in combination with other iTR activators or inhibitors of iTR inhibitors as described herein. In some embodiments, a small molecule is non-polymeric. In some embodiments, a small molecule is not an amino acid. In some embodiments, a small molecule is not a nucleotide. In some embodiments, a small molecule is not a saccharide.

The term “stabilization of the physiology of a metazoan or mammal” refers to the the use of life support technologies to maintain the viability of cells in a corresponding metazoan or mammal including a human wherein the physiological state of said metazoan or mammal would otherwise lead to the death of said cells in the postmortem state.

The term “subject” can be any metazoan animal. Often a subject is a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound described in the disclosure). The term “tissue damage” is used herein to refer to any type of damage or injury to cells, tissues, organs, or other body structures. The term encompasses, in various embodiments, degeneration due to disease, damage due to physical trauma or surgery, damage caused by exposure to deleterious substance, and other disruptions in the structure and/or functionality of cells, tissues, organs, or other body structures.

The term “tissue regeneration” or “TR” refers to at least partial regeneration, replacement, restoration, or regrowth of a tissue, organ, or other body structure, or portion thereof, following loss, damage, or degeneration, where said tissue regeneration but for the methods described in the present disclosure would not take place or would take place with an unacceptable level of fibrosis or scarring. Examples of tissue regeneration include the regrowth of severed digits, limbs, or skin the regrowth of normal functional cartilage, bone, muscle, tendons, and ligaments, the scarless regrowth of bone, cartilage, skin, or muscle that has been lost due to injury or disease, with an increase in size and cell number of an injured or diseased organ such that the tissue or organ approximates the normal size of the tissue or organ or its size prior to injury or disease. In addition, any normally non-regenerative somatic cell type can be targeted by the methods of the present invention, including tissues derived the principal embryonic germ layers endoderm, mesoderm, and ectoderm as described herein. Depending on the tissue type, tissue regeneration can occur via a variety of different mechanisms such as, for example, the rearrangement of pre-existing cells and/or tissue (e.g., through cell migration), the division of adult somatic stem cells or other progenitor cells and differentiation of at least some of their descendants, and/or the dedifferentiation, transdifferentiation, and/or proliferation of cells.

The term “TR activator” or “tissue regeneration activator” refers in iTR or induced tissue regeneration respectively.

The term “treat”, “treating”, “therapy”, “therapeutic” and similar terms in regard to a subject refer to providing medical and/or surgical management of the subject. Treatment can include, but is not limited to, administering a compound or composition (e.g., a pharmaceutical composition) to a subject. Treatment of a subject according to the instant disclosure is typically undertaken in an effort to promote regeneration, e.g., in a subject who has suffered tissue damage or is expected to suffer tissue damage (e.g., a subject who will undergo surgery). The effect of treatment can generally include increased regeneration, reduced scarring, and/or improved structural or functional outcome following tissue damage (as compared with the outcome in the absence of treatment), and/or can include reversal or reduction in severity or progression of a degenerative disease.

The term “variant” as applied to a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments a variant comprises at least one functional or structural domain, e.g., a domain identified as such in the Conserved Domain Database (CDD) of the National Center for Biotechnology Information (www.ncbi.nih.gov), e.g., an NCBI-curated domain. In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments, amino acid “substitutions” in a variant are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, praline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some embodiments, a substitution or deletion does not alter or delete an amino acid important for activity. Insertions or deletions may range in size from about 1 to 20 amino acids, e.g., I to IO amino acids. In some instances larger domains may be removed without substantially affecting function. In certain embodiments of the disclosure the sequence of a variant can be obtained by making no more than a total of 5, 10, 15, or 20 amino acid additions, deletions, or substitutions to the sequence of a naturally occurring enzyme. In some embodiments, no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

In some embodiments, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, 6×His, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4): 353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a polypeptide has a tag located at the N- or C-terminus, e.g., as an N- or C-terminal fusion. The polypeptide could comprise multiple tags. In some embodiments, a 6×His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some embodiments, a “self-cleaving” tag is used. See, e.g., PCT/US05/05763.

Sequences encoding a tag can be located 5′ or 3′ with respect to a polynucleotide encoding the polypeptide (or both). In some embodiments a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

In certain embodiments of the disclosure a fragment or variant, optionally excluding a heterologous portion, if present, possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A and Blundell, T L, J. Mol. Biol, 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue): W522-5, Jul. 1, 2004). Where embodiments of the disclosure relate to variants of a polypeptide, it will be understood that polynucleotides encoding the variant are provided.

The term “vector” is used herein to refer to a nucleic acid or a virus or portion thereof (e.g. a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA Useful nucleic acid vectors include, for example, DNA or RNA plasmids, cosmids, and naturally occurring or modified viral genomes or portions thereof or nucleic acids (DNA or RNA) that can be packaged into viral) capsids. The nucleic acids transported by said vector may encode one or more genes including concatenated genes. Plasmid vectors typically include an origin of replication and one or more selectable markers. Plasmids may include part or all of a viral genome (e.g., a viral promoter, enhancer, processing or packaging signals, etc.).

Viruses or portions thereof that can be used to introduce nucleic acid molecules into cells are referred to as viral vectors. Useful viral vectors include adenoviruses, adeno-associated viruses such as AAV2 and AAV9 or other serotypes of AAV and genetically-modified members of the AAV family of viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylations signals and/or a 3′untranslated regions. Vectors often include one or more appropriately positioned sites for restriction enzymes, to facilitate introduction into the vector of the nucleic acid to be expressed.

An expression vector comprises sufficient cis-acting elements for expression; other elements required or helpful for expression can be supplied by the host cell or in vitro expression system. Various techniques may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). Markers can be used for the identification and/or selection of cells that have taken up the vector and, typically, express the nucleic acid. Cells can be cultured in appropriate media to select such cells and, optionally, establish a stable cell line.

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

### In Vivo Decellularization and Subsequent Regenerative Recellularization

Numerous methods have been described for the decellularization of tissues ex vivo or in vivo (Mendibil U., et al, Tissue-Specific Decellularization Methods: Rationale and Strategies to Achieve Regenerative Compounds, Int. J Mal. Sci. 2020, 21, 5447; doi: 10.3390/ijms21155447 and Crapo, P. M., et al, An overview of tissue and whole organ decellularization processes. Biomaterials 2011, 32, 3233-3243) each incorporated by reference. These include the use of hypotonic solutions, mild surfactants such as, but not limited to Triton X-100, Triton X-200, deoxycholic acid, N-lauroylsarcosinate, SDS, CHAPS, DOC, SDC, and CHAPSO, that result in the lysis of cells in tissues of the body in situ while leaving the ECM of said tissue and the architecture of the tissue's ECM essentially intact. The aforementioned decellularization solutions may be applied to the tissue of interest by topical application to the tissue of interest, or more preferably, when the tissue is vascularized, by perfusing the tissue or organ by means of a pump through a vein or artery vascularizing that tissue or organ. Typically, the tissue or organ is first perfused with an anticoagulant such as heparin (2.0 U/g of tissue weight). The decellularization solution is then applied in the same manner as the anticoagulant, followed by rinse solution to remove cellular debris while leaving the ECM essentially intact. The rinse solutions include but are not limited to isotonic crystalloid solutions such as PBS, 0.9% sodium chloride solution, lactate Ringer's solution, and glucose solution or colloidal isotonic solutions incorporating starch of serum albumin. The removal of cellular debris can be facilitated by agitation or sonication applied to the tissue in vivo. The magnitude and frequency of the sonication may vary depending upon the anatomy and nature of the target tissue but by way of nonlimiting example, may approximate that of commonly used ultrasonic cleaners.

The methods of applying surfactants and the removal of cellular debris resulting from the lysis of cells in said tissue in vivo may vary from tissue to tissue as would be recognized by practitioners of the art. For example, a small nonvascularized tissue such as a tendon or ligament would be decellularized by applying a surfactant and rinse to the surface of said tissue while limiting the application to surrounding tissues wherein regeneration is not desired. In the case of cartilage, exposure to the tissue to chondroitinase ABC is useful in solubilizing glycoaminoglycans and thereby facilitating the removal of chondrocytes as described (Bautista, C. A., et al, Effects of Chondroitinase ABC-Mediated Proteoglycan Digestion on Decellularization and Recellularization of Articular Cartilage. PLoS ONE 2016, 11, e0158976) incorporated by reference. In addition surfactants such as Triton X-100 and SDS are useful in decellularization as described (Lee, D. J. et al, Decellularized bone matrix grafts for calvaria regeneration. J Tissue Eng. 2016, 7, 2041731416680306 and Elder, B. D. et al, Developing an articular cartilage decellularization process toward facet joint cartilage replacement. Neurosurgery 2010, 66, 722-727) each incorporated by reference. In the case of neural tissues such as the retina, wherein the tissue possesses a aqueous depot and is relatively isolated from other tissues of the body, said surfactant and subsequent rinse may be applied directly into the vitreous humor. In the case of the heart, useful detergents include SDS, sodium deoxycholate, PEG, or Triton X-100 for individual components as well as the entire heart. They are often used in combination (Seitzhaparova B, et al, Rabbit Heart Bioartificial Tissue: Perfusion Decellularization and Characterization. Biomed Phys Eng Express.

2024 Dec. 3. doi: 10.1088/2057-1976/ad99de. Epub ahead of print. PMID: 39626314) incorporated by reference. For example, for whole porcine heart decellularization 3% SDS may be utilized, optimally when the heart is perfused at a pressure between 90 and 120 mmHg. In the case of dermis, the tissue may be decellularized by perfusing with hypotonic buffer followed be detergents such as Triton X-100, DOC, N-lauroylsarcosinate, or SDS. Other protocols also include perfusion with proteases such as trypsin and/or dispase and EDTA (Chen, R N., et al, Process development of an acellular dermal matrix (ADM) for biomedical applications. Biomaterials 2004, 25, 2679-2686 and Farrokhi, A, et al, Evaluation of Detergent-Free and Detergent-Based Methods for Decellularization of Murine Skin. Tissue Eng. Part A 2018, 24, 955-967) incorporated by reference. In the case of vascularized tissues, said surfactant and rinse may be perfused via an artery or vein and drained from the corresponding vein or artery. In the case of thin tissues, CHAPS is preferred as a detergent given its relatively low permeability into tissue. In the case of neural tissue, Triton X-200 is preferred with perfusion with osmotic cell burst, or detergents such as SDS or Triton-I 00 without osmotic shock (Terenghi, G. Peripheral nerve injury and regeneration. Histol. Histopathol. 1995, 10, 709-718) incorporated by reference. Respiratory tissue such as lung or trachea may be decellularized by perfusion with SDS and Triton X-100 as previously described in mouse and rat cadaveric lung tissue (O'Neill, J. D., et al, Decellularization of human and porcine lung tissues for pulmonary tissue engineering. Ann. Thorac. Surg. 2013, 96, 1046-1056 and Gilpin, S. E., et al, Perfusion decellularization of human and porcine lungs: Bringing the matrix to clinical scale. J Hear. Lung Transplant. 2014, 33, 298-308) incorporated by reference. In the case of vascular tissue such as the saphenous vein, or major arteries such as the aorta, cerebral or pulmonary arteries, or the pulmonary vein as well as other vascular tissue, following the removal of blood cells by rinsing in PBS, detergents such as detergents such as SDS, EDTA, SDC, CHAPS, Triton X-100, or DOC have been reported to successfully decellularize the vessel when used individually or in combination (Cheng, J., et al, Combination of freeze-thaw with detergents: A promising approach to the decellularization of porcine carotid arteries. Biomed Mater. Eng. 2019, 30, 191-205) incorporated by reference. In the case of the gastrointestinal tract which includes derivatives of endodermal cells such as the esophagus, stomach, pancreas, liver, and intestines, perfusion perfusion with SDC, the use of hypotonic solutions have been reported (Maghsoudlou, P., et al, A decellularization methodology for the production of a natural acellular intestinal matrix. J Vis. Exp. 2013 and Kajbafzadeh, A. M., et al, Decellularized human fetal intestine as a bioscaffold for regeneration of the rabbit bladder submucosa. J Pediatr. Surg. 2018, 53, 1781-1788) each incorporated by reference. In the case of cornea tissue, many existing decellularization methods lead to an undesirable loss of transparency. Therefore, cornea is unique in it's need for a unique protocol. Methods described and readily available in the literature include the use of SDS in combination with the protease benzonase (Alio del Barrio, J. L., et al, Acellular human corneal matrix sheets seeded with human adipose-derived mesenchymal stem cells integrate functionally in an experimental animal model. Exp. Eye Res. 2015, 132, 91-100) or with the surfactantN-lauroyl glutamate combined with supernuclease (Liu, J., et al, Application of benzonase in preparation of decellularized lamellar porcinecorneal stroma for lamellar keratoplasty. J Biomed Mater. Res. Part A 2019, 107, 2547-2555) each of which are incorporated by reference. In general, tissues and whole organs may also be decellularized by the application of surfactants alone as described above or in combination with proteases such as trypsin, trypsin-EDTA, or pepsin, however these proteases have the potential to damage the ECM and therefore duration of exposure must be carefully controlled.

Said decellularized tissues have the advantage of providing a three-dimensional ECM that provides a niche for surrounding cells remaining in the tissue to migrate and thereby repopulate, preferably when said neighboring cells have been treated with iTR factors thereby reprogramming them into migratory and regenerative cells.

### TR Modulation Applied to Postmortem Animals

The present disclosure provides novel methods of applying iTR to a postmortem animal, more specifically, to cells, tissues, and organs of animals, more preferably mammalian animals, and most preferably postmortem humans.

In some aspects, the invention provides novel methods of enhancing regeneration comprising administering iTR factors to an entire metazoan organism such as a human. The applicants teach that primitive animals that display the potential for profound TR such as the regeneration of amputated limbs in axolotls, the regeneration of skin in the MRL or the African Spiny Mouse (Acomys), or the regeneration of whole body segments in planaria, do so by simply recapitulating normal embryonic development of the respective tissues.

Furthermore, the applicants teach that mortality is commonly the result of impaired organ function that can, through the use of the present invention, be repaired even after death by inducing the regenerative pathways expressed in the embryonic phases of development wherein the postmortem animal is maintained on life support or cryopreserved and resuscitated. Alternatively, the postmortem animal is not resuscitated but rather the cells, tissues, or organs of said postmortem animal are used in research or for therapeutic transplantation. The applicants further teach that the restoration of certain of these embryo-specific patterns of gene expression altered in the EFT in normally TR-resistant animals can induce competency for regeneration in any tissue, including responsiveness to organizing center factors, leading to complex tissue regeneration and a concommitant reduction in scar formation in organs intended to be utilized in transplantation. Lastly, the applicants teach novel agents and associated methods of inducing TR in postmortem animals. Said methods facilitate TR in mammalian species, particularly in the species Homo sapiens.

### Description of iTR Factors

Genes, RNAs, or proteins whose expression in embryonic phases of development facilitate scarless and epimorphic TR are herein designated “TR activators.” Molecules that alter the levels of TR activators in a manner leading to iTR are herein designated “iTR factors.” iTR genes and, the protein products of iTR genes, are often relatively conserved in animals ranging from sea anemones to mammals. The gene-encoded protein sequences, and sequences of nucleic acids (e.g., mRNA) encoding genes referred to herein, including those from a number of different non-human animal species are known in the art and can be found, e.g., in publicly available databases such as those available at the National Center for Biotechnology Information (NCBI) (www.ncbi.nih.gov).

The disclosure provides a number of different methods of modulating iTR genes in cells, tissues, and organs of postmortem animals. In general, an iTR factor can be, e.g., a small molecule, nucleic acid, oligonucleotide, polypeptide, peptide, lipid, carbohydrate, etc. The iTR factors referred to in the present invention include the global iTR activator genes: KLF4, SOX2, MYC; OCT4 or SOX2, OCT4, NANOG, and LIN28; or various combinations of: OCT4, SOX2, KLF4, NANOG, ESRRB, NR5A2, CEBPA, MYC, LIN28A and LIN28B, and diverse combinations of the genes such OCT4, SOX2, KLF4, and MYC; OCT4, KLF4, and LIN28A; OCT4, KLF4, LIN28A and TERT; OCT4, SOX2, KLF4, NANOG, MYC, and LIN28A and LIN28B and LIN28B LIN28A and LIN28B as their corresponding mRNAs or proteins, and including genetically-modified versions of aforementioned genes including but not limited to the substitution of SOX2 with genetically-modified SOX2 as described (Highly cooperative chimeric super-SOX induces naive pluripotency across species, Maccarthy, Caitlin M. et al. Cell Stem Cell, Volume 31, Issue 1, 127-147.e9).

Additional iTR factors include small molecule iTR factors such as the application to postmortem cells, tissues, and organs of small molecules to an affected tissue for 17 days being factors or combinations of the following factors: valproic acid at a concentration of 0.05-5.0 mM, preferably 0.5 mM; the GSK-3 inhibitor 6-[[2-[[4-(2,4-Dichlorophenyl)-5-(5-methyl-1H-imidazol-2-yl)-2-pyrimidinyl]amino]ethyl]amino]-3-pyridinecarbonitrile also known as CHIR99021 at a concentration of 7.0 nM-10 uM, preferably 10 uM; the inhibitor of the TGF [symbol] R-1/ALK5 2-(3-(6-Methylpyridine-2-yl)-1H-pyrazol-4-yl)-1,5-naphthyridine also known as RepSox at a concentration of 4.0 nM-10 uM, preferably 10 uM; the inhibitor of lysine-specific demethylase 1 Parnate (also named tranylcypromine) at a concentration of 2.0-10 uM, preferably 10 uM; the activator of adenylyl cyclase Forskolin at a concentration of 0.5-50 uM, preferably 50 uM; the retinoid receptor agonist arotinoid acid, otherwise known as 4-[(E)-2-(5,5,8,8-tetramethyl-6,7-dihydronaphthalen-2-yl) prop-1-enyl]benzoic acid, or TTNPB at a concentration of 1.0 nM-5 uM, preferably 5.0 uM;—then for 14 more days added to the cocktail are: the EZH2 inhibitor (1S,2R,5R)-5-(4-aminoimidazo[4,5-c]pyridin-1-yl)-3-(hydroxymethyl)cyclopent-3-ene-1,2-diol also known as 3-Deazaneplanocin A (DZNep) at a concentration of 0.05-0.24 uM, preferably 0.1 uM; then for the last seven days the aforementioned factors are discontinued and the inhibitor of the MEK/ERK pathway N-[(2R)-2,3-dihydroxypropoxy]-3,4-difluoro-2-(2-fluoro-4-iodoanilino)benzamide also known as PD0325901 is applied at a concentration of 0.1 nM-1.0 uM, preferably 1.0 uM and the GSK-3 inhibitor 6-[[2-[[4-(2,4-Dichlorophenyl)-5-(5-methyl-1H-imidazol-2-yl)-2-pyrimidinyl]amino]ethyl]amino]-3-pyridinecarbonitrile also known as CHIR99021 at a concentration of 7.0 nM-10 uM, preferably 10 uM, all the aforementioned factors being formulated in a physiologically-compatible vehicle. Additional iTR factors include the expression of segmental iTR activators delivered to cells, tissues, or organs of postmortem animals via gene therapy, delivery of mRNA including chemically-modified mRNA to enhance stability, or delivery of protein from iTR activator genes including: WTAP, METTL3, METTL14, TERT and the metabolite L-2-HG, RNAi directed to the eraser FTO, and/or molecules inactivating IGFBP6 such as monoclonal antibody-based competitive inhibition using an adeno-associated virus gene therapy vectors to induce a regenerative phenotype in vivo in somatic cells or the corresponding tissues, IGF2BP1, IGF2BP2, IGF2BP3, LIN28A, LIN28B, H19, FIRRE, AC108142.1, ADGRVJ, AFF3, AGA, ALDH5A1, ALXJ, AMH, AQP7P1, AQP7P3, B4GALNT4, BAHDJ, BBOXJ, Cllor/35, Cl4orf39, CASC9, CBCAQH03 5, CBX2, CCDCl44NL, CHKB-CPTJB, CHRM3, CPAMD8, CPTJB, DLXI, DOC2GP, DPY19L2, DRDJIP, DSG2, F2RL2, FAM157A, FAM157B, FAR2P1, FAR2P3, FIRRE, FOXDJ, FOXD4L4, FSIP2, GDFJ, GRIN3B, H2BFXP, IGF2BPJ, L3MBTLJ, LIN28B, LINC00648, LINC00649, LINC01021, LINC02315, LMNBJ, LOC644919, LOC728755, LOC791120, MED15P9, MNJ, MNJ, NMLAD2, Nblal0527, OXTR, PAQR6, PCAT7, PCDHAJ0, PCDHAJJ, PCDHA2, PCDHA3, PCDHA4, PCDHA5, PCDHACJ, PCDHBJ0, PCDHB14, PCDHB16, PCDHB17, PCDHB2, PCDHB2, PCDHB5, PCDHB9, PCDHGB4, PCDHGB6, PKP3, PLPPR3, POTEE, POTEF, POU2FI, PRR5L, RAB3IP, RGPDJ, RGPD2, SIXJ, SLCOJA2, TCF3, TSPANJJ, TUBB2B, WDR72, WRN, WSBJ, ZCCHC18, ZNF497, and ZNF853, or the inhibition of iTR inhibitors which are therefore considered herein as iTR activators include inhibitors of the iTR delivered to cells, tissues, or organs of postmortem animals via antisense gene therapy or delivery of RNAi including chemically-modified RNAi to enhance stability, where the iTR inhibitory genes include single genes or combinations of genes including: ACAT2, ADIRF, ALS2CR11, ANKRD65, ANKRD7, BACE2, BHMT2, CJ0orfll, Cl8orf56, C22orf26, CADPS2, CALHM2, CAT, CCDCl25, CCDCl44B, CCDCl44B, CCDC36, CCDC89, CLDNJJ, COMT, COX7A1, CTSF, DDX43, DNAJC15, DYNLT3, EGFLAM, ELOVL6, ESPNL, FAM24B, FDPS, FGF7, FKBP9L, FLG-ASJ, FRGJB, GPAT2, GYPE, HENMTJ, HIST2H2BA, IAHJ, INSJGJ, IRAK4, KRBOXJ, LINC00654, LINC00839, LINC00865, LINC01116, LMNA, LOCJ00233156, LOC205251, LOC283788, LRRK2, MADA, MAP JO, MEG3, MEG8, MEG9, MIR4458HG, MIRLET7HG, NMLADLJ, NKAPL, PAX8-ASJ, PCDHGA12, PCDHGA2, PCDHGA6, PCDHGA7, PCDHGA9, PCDHGB3, PCDHGB5, PLPP7 (PAPPDC3), POMC, PRPH2, PRR34, PRR34-ASJ, PRSS3, PSMD5, PTCHD3, PTCHD3P1, RPJJ-134021.1, RP5-1043L13.1, RPS7, SHMTJ, SPESPJ, SVIL-ASJ, TEKT4P2, TRIM4, TSPYL5, USP32P1, ZNF280D, ZNF300P1, ZNF572, ZNF578, ZNF585B, ZNF736, and ZNF790-ASJ. By way of nonlimiting example, said inhibition may be accomplished via the administration of RNAi as described herein. Said iTR factors may be used in various combinations including cocktails of small molecule activators and global or segmental activators as described herein.

In some embodiments of the invention, iTR factors inhibit by decreasing the amount of TR inhibitor RNA produced by cells and/or by decreasing the level of activity of TR inhibitor genes.
In the case of targeting TR inhibitors, factors are identified and used in research and therapy that reduce the levels of the product of the TR inhibitor gene. Said TR inhibitor gene can be any one or combination of TR inhibitor genes such as COX7A1 or NAALADL1 (see PCT application no. PCT/US2017/036452, filed Jun. 7, 2017 and titled “Improved Methods for Detecting and Modulating the Embryonic Fetal Transition in Mammalian Species,” U.S. Pat. No. 10,961,531 patented on Mar. 30, 2021 and titled “Compositions and Methods for Induced Tissue Regeneration In Mammalian Species;” U.S. publication no. U.S. 2021/0180013A1 filed on Jun. 17, 2021 and titled “Improved Methods for Inducing Tissue Regeneration and Senolysis in Mammalian Cells; U.S. publication no. U.S. 2020/0306296Al1 filed on Mar. 27, 2020 and titled “Induced Tissue Regeneration Using Extracellular Vesicles”; PCT application no. PCT/US2022/018563 filed on Mar. 2, 2022 and titled “Methods and Compositions Used to Modify Chromatin Architecture to Regulate Phenotype in Aging and Cancer”; PCT application no. PCT/US2022/046737 filed on Oct. 14, 2022 and titled “Methods for the Temporal Regulation of Reprogramming Factors in Mammalian Cells”; and U.S. provisional application No. 63/256,286 filed on Oct. 15, 2021 and titled “Methods for Modulating the Regenerative Phenotype in Mammalian Cells” the contents of which is incorporated herein by reference) and Description of iTR Factors herein. The amount of TR inhibitor gene RNA can be decreased by inhibiting synthesis of TR inhibitor RNA synthesis by cells (also referred to as “inhibiting TR inhibitor gene expression”), e.g., by reducing the amount of mRNA encoding TR inhibitor genes or by reducing translation of mRNA encoding TR inhibitor genes. Said factor can be by way of nonlimiting example, RNAi targeting a sequence within the TR inhibitor genes such as COX7A1 or NAALADLJ (see PCT application no. PCT/US2017/036452, filed Jun. 7, 2017 and titled “Improved Methods for Detecting and Modulating the Embryonic Fetal Transition in Mammalian Species, and U.S. patent no. U.S. Pat. No. 10,961,531 patented on Mar. 30, 2021 “Compositions and Methods for Induced Tissue Regeneration in Mammalian Species;” the contents of which is incorporated herein by reference).

In some embodiments, TR inhibitor gene expression is inhibited by RNA interference (RNAi). As known in the art, RNAi is a process in which the presence in a cell of double stranded RNA that has sequence correspondence to a gene leads to sequence-specific inhibition of the expression of the gene, typically as a result of cleavage or translational repression of the mRNA transcribed from the gene. Compounds useful for causing inhibition of expression by RNAi (“RNAi agents”) include short interfering RNAs (siRNAs), short hairpin RNAs (shRNAs), microRNAs (miRNAs), and miRNA-like molecules.

One of skill in the art can readily design sequences for RNAi agents, e.g., siRNAs, useful for inhibiting expression of mammalian TR inhibitor genes, e.g., human TR inhibitor genes once one has identified said TR inhibitor genes. In some embodiments, such sequences are selected to minimize “off-target” effects. For example, a sequence that is complementary to a sequence present in TR inhibitor gene mRNA and not present in other mRNAs expressed in a species of interest (or not present in the genome of the species of interest) may be used. Position-specific chemical modifications may be used to reduce potential off-target effects. In some embodiments, at least two different RNAi agents, e.g., siRNAs, targeted to TR inhibitor gene mRNA are used in combination. In some embodiments, a microRNA (which may be an artificially designed microRNA) is used to inhibit TR inhibitor gene expression.

In some embodiments of the invention, TR inhibitor gene expression is inhibited using an antisense molecule comprising a single-stranded oligonucleotide that is perfectly or substantially complementary to mRNA encoding TR inhibitor genes. The oligonucleotide hybridizes to TR inhibitor gene mRNA leading, e.g., to degradation of the mRNA by RNase Hor blocking of translation by steric hindrance. In other embodiments of the invention, TR inhibitor gene expression is inhibited using a ribozyme or triplex nucleic acid.

In some embodiments, of the invention, a TR inhibitor inhibits at least one activity of an TR inhibitor protein. TR inhibitor activity can be decreased by contacting the TR inhibitor protein with a compound that physically interacts with the TR inhibitor protein. Such a compound may, for example, alter the structure of the TR inhibitor protein (e.g., by covalently modifying it) and/or block the interaction of the TR inhibitor protein with one or more other molecule(s) such as cofactors or substrates. In some embodiments, inhibition or reduction may be a decrease of at least about 5%, 10%, 15%, 20%, 25%, 30%, 35%, 40%, 45%, 50%, 55%, 60%, 65%, 70%, 75%, 80%, 85%, 90%, 95%, or 99% of a reference level (e.g., a control level). A control level may be the level of the TR inhibitor that occurs in the absence of the factor. For example, a TR factor may reduce the level of the TR inhibitor protein to no more than 95%, 90%, 85%, 80%, 75%, 70%, 65%, 60%, 55%, 50%, 40%, 30%, 25%, 20%, 10%, or 5% of the level that occurs in the absence of the factor under the conditions tested. In some embodiments, levels of the TR inhibitor are reduced to 75% or less of the level that occurs in the absence of the factor, under the conditions tested. In some embodiments, levels of the TR inhibitor are reduced to 50% or less of the level that occurs in the absence of the TR factor, under the conditions tested. In some embodiments, levels of the TR inhibitor are reduced to 25% or less of the level that occurs in the absence of the iTR factor, under the conditions tested. In some embodiments, levels of the TR inhibitor are reduced to 10% or less of the level that occurs in the absence of the iTR factor, under the conditions tested. In some cases the level of modulation (e.g., inhibition or reduction) as compared with a control level is statistically significant. As used herein, “statistically significant” refers to a p-value of less than 0.05, e.g., a p-value of less than 0.025 or a p-value of less than 0.01, using an appropriate statistical test (e.g, ANOVA, t-test, etc.).

In some embodiments of the invention, a compound directly inhibits TR inhibitor proteins, i.e., the compound inhibits TR inhibitor proteins by a mechanism that involves a physical interaction (binding) between the TR inhibitor and the iTR factor. For example, binding of a TR inhibitor to an iTR factor can interfere with the TR inhibitor's ability to catalyze a reaction and/or can occlude the TR inhibitors active site. A variety of compounds can be used to directly inhibit TR inhibitors. Exemplary compounds that directly inhibit TR inhibitors can be, e.g., small molecules, antibodies, or aptamers.

In some embodiments of the invention, an iTR factor binds covalently to the TR inhibitor.

For example, the compound may modify amino acid residue(s) that are needed for enzymatic activity. In some embodiments, an iTR factor comprises one or more reactive functional groups such as an aldehyde, haloalkane, alkene, fluorophosphonate (e.g., alkyl fluorophosphonate), Michael acceptor, phenyl sulfonate, methylketone, e.g., a halogenated methylketone or diazomethylketone, fluorophosphonate, vinyl ester, vinyl sulfone, or vinyl sulfonamide, that reacts with an amino acid side chain of TR inhibitors. In some embodiments, an iTR factor inhibitor comprises a compound that physically interacts with a TR inhibitor, wherein the compound comprises a reactive functional group. In some embodiments, the structure of a compound that physically interacts with the TR inhibitor is modified to incorporate a reactive functional group. In some embodiments, the compound comprises a TR inhibitor substrate analog or transition state analog. In some embodiments, the compound interacts with the TR inhibitor in or near the TR inhibitor active site.

In other embodiments, an iTR factor binds non-covalently to a TR inhibitor and/or to a complex containing the TR inhibitor and a TR inhibitor substrate. In some embodiments, an iTR factor binds non-covalently to the active site of a TR inhibitor and/or competes with substrate(s) for access to the TR inhibitor active site. In some embodiments, an iTR factor binds to the TR inhibitor with a effective dose of approximately 10⁻³ Mor less, e.g., 10⁻⁴M or less, e.g., 10⁻⁵ Mor less, e.g., 10⁻⁶ Mor less, 10⁻⁷ Mor less, 10⁻⁸ Mor less, or 10⁻⁹ M or less under the conditions tested, e.g., in a physiologically acceptable solution such as phosphate buffered saline. Binding affinity can be measured, e.g., using surface plasmon resonance (e.g., with a Biacore system), isothermal titration calorimetry, or a competitive binding assay, as known in the art. In some embodiments, the inhibitor comprises a TR inhibitor substrate analog or transition state analog. In the case of increasing the activity of TR activators, any combination of the genes OCT4, SOX2, KLF4, NANOG, ESRRB, NR5A2, CEBPA, MYC, LIN28A, TERT, and LIN28 or their respective RNAs or proteins and including genetically-modified versions of aforementioned genes including but not limited to modified SOX2 as described (Highly cooperative chimeric super-SOX induces naive pluripotency across species, Maccarthy, Caitlin M. et al. Cell Stem Cell, Volume 31, Issue 1, 127-147.e9) may be used. The levels of the products of these genes may be introduced using the vectors described herein.

In other embodiments, the iTR factors are constructs that introduce RNA into cells, tissues, and organs of postmortem animals. either directly or through gene expression constructs that are capable of inducing pluripotency if allowed to react with cells for a sufficient period of time, but for lesser times can cause iTR. Preferably, the RNAs do not include all the RNAs needed for reprogramming to pluripotency and instead include only LIN28A or LIN28B optionally together with an agent to increase telomere length such as RNA for the catalytic component of telomerase (TERI). Most preferably, the agents to induce iTR are genes/factors induced by LIN28A or—encoded proteins such as GFER, optionally in combination with an agent that increases telomere length such as the RNA or gene encoding TERT, and/or in combination with the factors disclosed herein important for iTR such as 0.05-SmM valproic acid, preferably 0.5 mM valproic acid, 1-100 ng/ml AMH, preferably 10 ng/mL AMH, and 2-200 ng/mL GFER, preferably 20 ng/mL. When administered in vivo, such factors are preferably administered in a slow-release hydrogel matrix such as one comprised of chemically modified and crosslinked hyaluronic acid and collagen such as HyStem matrices.

### TR Activator Polypeptides and Nucleic Acids

TR activators include combinations of agents under the headings “Embryonic Markers” and “Fetal/Adult Markers”, respectively. TR activator and TR inhibitor polypeptides useful in the inventive methods may be obtained by a variety of methods. In some embodiments, the polypeptides are produced using recombinant DNA techniques. Standard methods for recombinant protein expression can be used. A nucleic acid encoding a TR activator or TR inhibitor gene can readily be obtained, e.g., from cells that express the genes (e.g., by PCR or other amplification methods or by cloning) or by chemical synthesis or in vitro transcription based on the cDNA sequence polypeptide sequence. One of ordinary skill in the art would know that due to the degeneracy of the genetic code, the genes can be encoded by many different nucleic acid sequences. Optionally, a sequence is codon-optimized for expression in a host cell of choice. The genes could be expressed in bacterial, fungal, animal, or plant cells or organisms. The genes could be isolated from cells that naturally express it or from cells into which a nucleic acid encoding the protein has been transiently or stably introduced, e.g., cells that contain an expression vector encoding the genes. In some embodiments, the gene is secreted by cells in culture and isolated from the culture medium.

In some embodiments of the invention, the sequence of a TR activator or TR inhibitor polypeptide is used in the inventive screening methods. A naturally occurring TR activator or TR inhibitor polypeptide can be from any species whose genome encodes a TR activator or TR inhibitor polypeptide, e.g., human, non-human primate, rodent, etc. A polypeptide whose sequence is identical to naturally occurring TR activator or TR inhibitor is sometimes referred to herein as “native TR activator/inhibitor”. A TR activator or TR inhibitor polypeptide of use in the invention may or may not comprise a secretion signal sequence or a portion thereof. For example, mature TR activator or TR inhibitor comprising or consisting of amino acids 20-496 of human TR activator or TR inhibitor (or corresponding amino acids of TR activator or TR inhibitor of a different species) may be used.

In some embodiments, a polypeptide comprising or consisting of a variant or fragment of TR activator or TR inhibitor is used. TR activator or TR inhibitor variants include polypeptides that differ by one or more amino acid substitutions, additions, or deletions, relative to TR activator or TR inhibitor. In some embodiments, a TR activator or TR inhibitor variant comprises a polypeptide at least 70%, 75%, 80%, 85%, 90%, 95%, 96%, 97%, 98%, 99%, or more identical to at least amino acids 20-496 of TR activator or TR inhibitor (e.g., from human or mouse) over at least 50%, 60%, 70%, 80%, 85%, 90%, 95%, 96%, 97%, 98%, 99%, or 100% of at least amino acids 20-496 of human TR activator or TR inhibitor or amino acids 20-503 of mouse TR activator or TR inhibitor. In some embodiments, a TR activator or TR inhibitor variant comprises a polypeptide at least 80%, 85%, 90%, 95%, 96%, 97%, 98%, 99%, or more identical to at least amino acids 20-496 of human TR activator or TR inhibitor or amino acids 20-503 of mouse TR activator or TR inhibitor. In some embodiments, a TR activator or TR inhibitor polypeptide comprises a polypeptide at least 80%, 85%, 90%, 95%, 96%, 97%, 98%, 99%, or more identical to at least amino acids 20-496 of human TR activator or TR inhibitor or amino acids 20-503 of mouse TR activator or TR inhibitor. A nucleic acid that encodes a TR activator or TR inhibitor variant or fragment can readily be generated, e.g., by modifying the DNA that encodes native TR activator or TR inhibitor using, e.g., site-directed mutagenesis, or by other standard methods, and used to produce the TR activator or TR inhibitor variant or fragment. For example, a fusion protein can be produced by cloning sequences that encode TR activator or TR inhibitor into a vector that provides the sequence encoding the heterologous portion. In some embodiments a tagged TR activator or TR inhibitor is used. For example, in some embodiments a TR activator or TR inhibitor polypeptide comprising a 6×His tag, e.g., at its C terminus, is used.

### Test Compounds

A wide variety of test compounds can be used in the inventive methods for identifying iTR factors and global activators of iTR. For example, a test compound can be a small molecule, polypeptide, peptide, nucleic acid, oligonucleotide, lipid, carbohydrate, antibody, or hybrid molecule including but not limited to those described herein, including mRNA for the genes OCT4, SOX2, KLF4, NANOG, ESRRB, NR5A2, CEBPA, MYC, LIN28A and LIN28B and including genetically-modified versions of aforementioned RNA including but not limited to modified SOX2 as described (Highly cooperative chimeric super-SOX induces naive pluripotency across species, Maccarthy, Caitlin M. et al. Cell Stem Cell, Volume 31, Issue 1, 127-147.e9) alone and in diverse combinations, and in diverse combinations with small molecule compounds such as combinations of the following compounds: inhibitors of glycogen synthase 3 (GSK3) including but not limited to CHIR99021; inhibitors of TGF-beta signaling including but not limited to SB431542, A-83-01, and E616452; HDAC inhibitors including but not limited to aliphatic acid compounds including but not limited to: valproic acid, phenylbutyrate, and nbutyrate; cyclic tetrapeptides including trapoxin B and the depsipeptides; hydroxamic acids such as trichostatin A, vorinostat (SAHA), belinostat (PXD101), LAQ824, panobinostat (LBH589), and the benzamides entinostat (MS-275), CI994, mocetinostat (MGCD0103); those specifically targeting Class I (HDAC1, HDAC2, HDAC3, and HDAC8), IIA (HDAC4, HDAC5, HDAC7, and HDAC9), IIB (HDAC6 and HDACIO), PI (SIRT1, SIRT2, SIRT3, SIRT4, SIRT5, SIRT6, or SIRT7) including the sirtuin inhibitors nicotinomide, diverse derivatives ofNAD, dihydrocoumarin, naphthopyranone, and 2-hydroxynaphthaldehydes, or IV (HDAC11) deacetylases; inhibitors of H3K4/9 histone demethylase LSD1 including but not limited to pamate; inhibitors of Dot1L including but not limited to EPZ004777; inhibitors of G9a including but not limited to Bix01294; inhibitors of EZH2 including but not limited to DZNep, inhibitors of DNA methyltransferase including but not limited to RGI08; 5-aza-2′deoxycytidine (trade name Vidaza and Azadine); vitamin C which can inhibit DNA methylation, increase Tetl which increases 5hmC which is a first step of demethylation; activators of 3′ phosphoinositide-dependent kinase I including but not limited to PS48; promoters of glycolysis including but not limited to Quercetin and fructose 2,6-bisphosphate (an activator of phosphofructokinase I); agents that promote the activity of the HIFI transcription complex including but not limited to Quercetin; RAR agonists including but not limited to AM580, CD437, and TTNPB; agents that mimic hypoxia including but not limited to Resveratrol; agents that increase telomerase activity including but not limited to the exogenous expression of the catalytic component of telomerase (TERI), agents that promote epigenetic modifications via downregulation of LSD I, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or NAALADL1, or other inhibitors of iTR as described herein, and/or assaying for increased expression of PCDHB2 or AMH or other activators or iTR as described herein, or in injured or diseased tissues in vivo, or in modulating the lifespan of animals in vivo.

Monitoring the state of regeneration in cells, tissues, and organs reprogrammed in postmortem animals as described in the present invention. Useful assays include monitoring the level of gene expression in cells wherein said gene expression distinguishes the embryonic state of development for that cell from the adult state of development for that cell. These iTR markers include COX7A1, PLPP7, and NAALADL1 as well as gene expression or protein markers of pluripotency including DNMT3B, and HELLS or Tra-1-60, Tra-1-81, and SSEA4 respectively are performed to optimize global patterns of iTR gene expression without reverting the target cells to pluripotency. Examples of individual agents and combinations of agents screened are the genes: OCT4, SOX2, KLF4, MYC and LIN28A; OCT4; KLF4; OCT4, KLF4; OCT4, KLF4, LIN28A; OCT4, KLF4, LIN28B; SOX2; MYC; NANOG; ESRRB; NT5A2; OCT4, SOX2, KLF4, and LIN28A; OCT4, SOX2, KLF4, and LIN28B; OCT4, KLF4, MYC and LIN28A; the mRNAs or proteins corresponding to aforementioned genes, and including genetically-modified versions of aforementioned genes including but not limited to modified SOX2 as described (Highly cooperative chimeric super-SOX induces naive pluripotency across species, Maccarthy, Caitlin M. et al. Cell Stem Cell, Volume 31, Issue 1, 127-147.e9), and each of the preceding combinations of agents together with 0.25 mM NaB, 5 mM PS48 and 0.5 mM A-83-01 during the first four weeks, followed by treatment with 0.25 mM sodium butyrate, 5 mM PS48, 0.5 mM A-83-01 and 0.5 mM PD0325901 each of which is assayed at 0, 1, 2, 4, 7, 10, and 14 days for markers of global modulation of iTR gene expression.

Compounds can be obtained from natural sources or produced synthetically. Compounds can be at least partially pure or may be present in extracts or other types of mixtures whose components are at least in part unknown or uncharacterized. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multiwell plates (e.g., 384 well plates, 1596 well plates, etc.). They can be dissolved in a solvent (e.g., DMSO) or provided in dry form, e.g., as a powder or solid. Collections of synthetic, semi-synthetic, and/or naturally occurring compounds can be tested. Compound libraries can comprise structurally related, structurally diverse, or structurally unrelated compounds. Compounds may be artificial (having a structure invented by man and not found in nature) or naturally occurring. In some embodiments, a library comprises at least some compounds that have been identified as “hits” or “leads” in other drug discovery programs and/or derivatives thereof. A compound library can comprise natural products and/or compounds generated using non-directed or directed synthetic organic chemistry. Often a compound library is a small molecule library. Other libraries of interest include peptide or peptoid libraries, cDNA libraries, antibody libraries, and oligonucleotide libraries. A library can be focused (e.g., composed primarily of compounds having the same core structure, derived from the same precursor, or having at least one biochemical activity in common).

Compound libraries are available from a number of commercial vendors such as Tocris Bioscience, Nanosyn, BioFocus, and from government entities. For example, the Molecular Libraries Small Molecule Repository (MLSMR), a component of the U.S. National Institutes of Health (NIH) Molecular Libraries Program is designed to identify, acquire, maintain, and distribute a collection of >300,000 chemically diverse compounds with known and unknown biological activities for use, e.g., in high-throughput screening (HTS) assays (see https://mli.nih.gov/mli/). The NIH Clinical Collection (NCC) is a plated array of approximately 450 small molecules that have a history of use in human clinical trials. These compounds are highly drug-like with known safety profiles. In some embodiments, a collection of compounds comprising “approved human drugs” is tested. An “approved human drug” is a compound that has been approved for use in treating humans by a government regulatory agency such as the US Food and Drug Administration, European Medicines Evaluation Agency, or a similar agency responsible for evaluating at least the safety of therapeutic agents prior to allowing them to be marketed. The test compound may be, e.g., an antineoplastic, antibacterial, antiviral, antifungal, antiprotozoal, antiparasitic, antidepressant, antipsychotic, anesthetic, antianginal, antihypertensive, antiarrhythmic, anti-inflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments, a compound is one that has undergone at least some preclinical or clinical development or has been determined or predicted to have “drug-like” properties. For example, the test compound may have completed a Phase I trial or at least a preclinical study in non-human animals and shown evidence of safety and tolerability.

In some embodiments, a test compound is substantially non-toxic to cells of an organism to which the compound may be administered and/or to cells with which the compound may be tested, at the concentration to be used or, in some embodiments, at concentrations up to IO-fold, 100-fold, or 1,000-fold higher than the concentration to be used. For example, there may be no statistically significant effect on cell viability and/or proliferation, or the reduction in viability or proliferation can be no more than 1%, 5%, or 10% in various embodiments. Cytotoxicity and/or effect on cell proliferation can be assessed using any of a variety of assays. For example, a cellular metabolism assay such as AlamarBlue, MTT, MTS, XTT, and CellTitre Glo assays, a cell membrane integrity assay, a cellular ATP-based viability assay, a mitochondrial reductase activity assay, a BrdU, EdU, or H3-Thymidine incorporation assay could be used. In some embodiments, a test compound is not a compound that is found in a cell culture medium known or used in the art, e.g., culture medium suitable for culturing vertebrate, e.g., mammalian cells or, if the test compound is a compound that is found in a cell culture medium known or used in the art, the test compound is used at a different, e.g., higher, concentration when used in a method of the present invention.

### Assays for Global Activators of iTR in Cells, Tissues, and Organs of Postmortem Animals: Aspects of Assay Implementation and Controls

Various inventive screening assays described above involve determining whether a test iTR factor or combination of factors generate iTR-modified cells, tissues, and organs of postmortem animals. Suitable cells for expression of a reporter molecule are described above. In performing an inventive assay, assay components (e.g., cells, TR activator or TR inhibitor polypeptide, and test compounds) are typically dispensed into multiple vessels or other containers. In the case of postmortem animals, iTR factors may be injected into multiple sites of the animal and excised at various time points to assay for alteration of iTR-related gene expression such as the expression of iTR activator genes or reduction of the expression of iTR inhibitory genes where said genes were not the genes injected into the tissue. For high throughput assays, any type of vessel or article capable of containing cells can be used. In many embodiments of the invention, the vessels are wells of a multi-well plate (also called a “microwell plate”, “microtiter plate”, etc. For purposes of description, the term “well” will be used to refer to any type of vessel or article that can be used to perform an inventive screen, e.g., any vessel or article that can contain the assay components. It should be understood that the invention is not limited to use of wells or to use of multi-well plates. In some embodiments, any article of manufacture in which multiple physically separated cavities (or other confining features) are present in or on a substrate can be used. For example, assay components can be confined in fluid droplets, which may optionally be arrayed on a surface and, optionally, separated by a water resistant substance that confines the droplets to discrete locations, in channels of a microfluidic device, etc.

In general, assay components can be added to wells in any order. For example, cells, tissues, and organs of postmortem animals can be added first and maintained in culture for a selected time period (e.g., between 6 and 48 hours) prior to addition of a test compound and target TR activator. In some embodiments, compounds are added to wells prior to addition of polypeptides of cells. In some embodiments, expression of a reporter polypeptide is induced after plating the cells, optionally after addition of a test compound to a well. In some embodiments, expression of the reporter molecule is achieved by transfecting the cells with an expression vector that encodes the reporter polypeptide. In some embodiments, the cells have previously been genetically engineered to express the reporter polypeptide. In some embodiments, expression of the reporter molecule is under control of regulatable expression control elements, and induction of expression of the reporter molecule is achieved by contacting the cells with an agent that induces (or derepresses) expression. The assay composition comprising cells, test compound, or polypeptide is maintained for a suitable time period during which test compound may (in the absence of a test compound that inhibits its activity) cause an increase or decrease of the level or activity of the target TR activator or TR inhibitor. The number of cells, amount of TR activator or TR inhibitor polypeptide, and amount of test compound to be added will depend, e.g., on factors such as the size of the vessel, cell type, and can be determined by one of ordinary skill in the art. In some embodiments, the ratio of the molar concentration of TR activator or TR inhibitor polypeptide to test compound is between 1:10 and 10:1. In some embodiments, the number of cells, amount of test compound, and length of time for which the composition is maintained can be selected so that a readily detectable level signal after a selected time period in the absence of a test compound. In some embodiments, cells are at a confluence of about 25%-75%, e.g., about 50%, at the time of addition of compounds. In some embodiments, between 1,000 and 10,000 cells/well (e.g., about 5,000 cells/well) are plated in about 100 mi medium per well in 96-well plates. In other exemplary embodiments, cells are seeded in about 301-50 mi of medium at between 500 and 2,000 (e.g., about 1000) cells per well into 384-well plates. In some embodiments, compounds are tested at multiple concentrations (e.g., 2-10 different concentrations) and/or in multiple replicates (e.g., 2-10 replicates). Multiple replicates of some or all different concentrations can be performed. In some embodiments, candidate TR factors are used at a concentration between 0.1 mg/ml and 100 mg/ml, e.g., 1 mg/ml and 10 mg/ml. In some embodiments, candidate TR factors are used at multiple concentrations. In some embodiments, compounds are added to cells between 6 hours and one day (24 hr) after seeding.

In some aspects of any of the inventive compound screening and/or characterization methods, a test compound is added to an assay composition in an amount sufficient to achieve a predetermined concentration. In some embodiments the concentration is up to about 1 nM. In some embodiments the concentration is between about 1 nM and about 100 nM. In some embodiments the concentration is between about 100 nM and about 10 mM. In some embodiments the concentration is at least 10 mM, e.g., between 10 mM and 100 mM. The assay composition can be maintained for various periods of time following addition of the last component thereof. In certain embodiments the assay composition is maintained for between about 10 minutes and about 4 days, e.g., between 1 hour and 3 days, e.g., between 2 hours and 2 days, or any intervening range or particular value, e.g., about 4-8 hours, after addition of all components. Multiple different time points can be tested.

Additional aliquots of test compound can be added to the assay composition within such time period. In some embodiments, cells are maintained in cell culture medium appropriate for culturing cells of that type. In some embodiments, a serum-free medium is used. In some embodiments, the assay composition comprises a physiologically acceptable liquid that is compatible with maintaining integrity of the cell membrane and, optionally, cell viability, instead of cell culture medium. Any suitable liquid could be used provided it has the proper osmolarity and is otherwise compatible with maintaining reasonable integrity of the cell membrane and, optionally, cell viability, for at least a sufficient period of time to perform an assay. One or more measurements indicative of an increase in the level of active TR activator or decrease in TR inhibitor can be made during or following the incubation period.

In some embodiments, the compounds screened for potential to be global activators of iTR are chosen from agents capable in other conditions of inducing pluripotency in somatic cell types. Such agents include the following compounds individually or in combination: the genes OCT4, SOX2, KLF4, NANOG, ESRRB, NR5A2, CEBPA, TERT, MYC, LIN28A and LIN28B and including genetically-modified versions of aforementioned genes including but not limited to modified SOX2 as described (Highly cooperative chimeric super-SOX induces naive pluripotency across species, Maccarthy, Caitlin M. et al. Cell Stem Cell, Volume 31, Issue 1, 127-147.e9) alone and in combination with small molecule compounds such as combinations with small molecular activators of iTR such as the following compounds: inhibitors of glycogen synthase 3 (GSK3) including but not limited to CHIR99021; inhibitors of TGF-beta signaling including but not limited to SB43 1542, A-83-01, and E616452; HDAC inhibitors including but not limited to aliphatic acid compounds including but not limited to: valproic acid, phenylbutyrate, and n-butyrate; cyclic tetrapeptides including trapoxin Band the depsipeptides; hydroxamic acids such as trichostatin A, vorinostat (SAHA), belinostat (PXDIOI), LAQ824, panobinostat (LBH589), and the benzamides entinostat (MS-275), CI994, mocetinostat (MGCD0103); those specifically targeting Class I (HDAC1, HDAC2, HDAC3, and HDAC8), PA (HDAC4, HDAC5, HDAC7, and HDAC9), IIB (HDAC6 and HDACIO), III (SIRT1, SIRT2, SIRT3, SIRT4, SIRT5, SIRT6, or SIRT7) including the sirtuin inhibitors nicotinomide, diverse derivatives of NAD, dihydrocoumarin, naphthopyranone, and 2-hydroxynaphthaldehydes, or IV (HDACl 1) deacetylases; inhibitors ofH3K4/9 histone demethylase LSD I including but not limited to parnate; inhibitors of DotlL including but not limited to EPZ004777; inhibitors of G9a including but not limited to Bix01294; inhibitors ofEZH2 including but not limited to DZNep, inhibitors of DNA methyltransferase including but not limited to RGI 08; 5-aza-2′deoxycytidine (trade name Vidaza and Azadine); vitamin C which can inhibit DNA methylation, increase Tetl which increases 5hmC which is a first step of demethylation; activators of 3′ phosphoinositide-dependent kinase I including but not limited to PS48; promoters of glycolysis including but not limited to Quercetin and fructose 2,6-bisphosphate (an activator of phosphofructokinase I); agents that promote the activity of the HIFI transcription complex including but not limited to Quercetin; RAR agonists including but not limited to AM580, CD437, and TTNPB; agents that mimic hypoxia including but not limited to Resveratrol; agents that increase telomerase activity including but not limited to the exogenous expression of the catalytic component of telomerase (TERT), agents that promote epigenetic modifications via downregulation of LSD I, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or NMLADLJ, or other inhibitors of iTR as described herein, and/or assaying for increased expression of PCDHB2 or AMH or other activators or iTR as described herein, or in injured or diseased tissues if postmortem animals, for use in transplantation or in modulating the lifespan of animals in which said cells, tissues, or organs are transplanted.

In some embodiments, individual compounds, each typically of known identity (e.g., structure and/or sequence), are added to each of a multiplicity of wells. In some embodiments, two or more compounds may be added to one or more wells. In some embodiments, one or more compounds of unknown identity may be tested. The identity may be determined subsequently using methods known in the art.

In various embodiments, foregoing assay methods of the invention are amenable to high throughput screening (HTS) implementations. In some embodiments, the screening assays of the invention are high throughput or ultra high throughput (see, e.g., Fernandes, P. B., Curr Opin Chem. Biol. 1998, 2:597; Sundberg, S A, Curr Opin Biotechnol. 2000, 11:47). High throughput screens (HTS) often involve testing large numbers of compounds with high efficiency, e.g., in parallel. For example, tens or hundreds of thousands of compounds can be routinely screened in short periods of time, e.g, hours to days. In some embodiments, HTS refers to testing of between 1,000 and 100,000 compounds per day. In some embodiments, ultra high throughput refers to screening in excess of 100,000 compounds per day, e.g., up to 1 million or more compounds per day. The screening assays of the invention may be carried out in a multi-well format, for example, a 96-well, 384-well format, 1,536-well format, or 3,456-well format and are suitable for automation. In some embodiments, each well of a microwell plate can be used to run a separate assay against a different test compound or, if concentration or incubation time effects are to be observed, a plurality of wells can contain test samples of a single compound, with at least some wells optionally being left empty or used as controls or replicates. Typically, HTS implementations of the assays disclosed herein involve the use of automation. In some embodiments, an integrated robot system including one or more robots transports assay microwell plates between multiple assay stations for compound, cell and/or reagent addition, mixing, incubation, and readout or detection. In some aspects, an HTS system of the invention may prepare, incubate, and analyze many plates simultaneously. Suitable data processing and control software may be employed. High throughput screening implementations are well known in the art. Without limiting the invention in any way, certain general principles and techniques that may be applied in embodiments of a HTS of the present invention are described in Macarron R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006).

An additional compound may, for example, have one or more improved pharmacokinetic and/or pharmacodynamic properties as compared with an initial hit or may simply have a different structure. An “improved property” may, for example, render a compound more effective or more suitable for one or more purposes described herein. In some embodiments, for example, a compound may have higher affinity for the molecular target of interest (e.g., TR activator or TR inhibitor gene products), lower affinity for a non-target molecule, greater solubility (e.g., increased aqueous solubility), increased stability (e.g., in blood, plasma, and/or in the gastrointestinal tract), increased half-life in the body, increased bioavailability, and/or reduced side effect(s), etc. Optimization can be accomplished through empirical modification of the hit structure (e.g., synthesizing compounds with related structures and testing them in cell-free or cell-based assays or in non-human animals) and/or using computational approaches. Such modification can in some embodiments make use of established principles of medicinal chemistry to predictably alter one or more properties. In some embodiments, one or more compounds that are “hit” are identified and subjected to systematic structural alteration to create a second library of compounds (e.g., refined lead compounds) structurally related to the hit. The second library can then be screened using any of the methods described herein. In some embodiments, an iTR factor is modified or incorporates a moiety that enhances stability (e.g., in serum), increases half-life, reduces toxicity or immunogenicity, or otherwise confers a desirable property on the compound.

### RNAi

By way of nonlimiting example, dsRNA is prepared from in vitro transcription reactions (Promega) using PCR-generated templates with flanking T7 promoters, purified by phenol extraction and ethanol precipitation, and annealed after resuspension in water. Intact experimental animals are injected with 4×30 nL dsRNA on three consecutive days following induced tissue injury beginning with the first injection two hours after surgery.
iTR markers genes are those genes that normally activate or inhibit the regenerative state and are useful for measuring or assaying for the extent of induction of the regenerative state in the practice of the present invention. iTR marker genes are genes commonly up- or down-regulated in cells in the embryonic/regenerative state compared to the corresponding genes of cells in the adult/nonregenerative state. Said iTR genes can be any one or combination of said genes (see PCT application no. PCT/US2017/036452, filed Jun. 7, 2017 and titled “Improved Methods for Detecting and Modulating the Embryonic Fetal Transition in Mammalian Species,” U.S. patent no. U.S. Pat. No. 10,961,531, patented on Mar. 30, 2021 “Differentially-methylated regions of the genome useful as markers of embryo-adult transitions,”;” U.S. published application no. 2022/0316013A1, filed on Aug. 25, 2020 and entitled “Differentially-Methylated Regions of the Genome Useful as Markers of Embryo-Adult Transitions”; U.S. provisional patent application No. 63/155,631, filed on Mar. 2, 2021 and titled “Use of Protocadherins in Methods of Diagnosing and Treating Cancer”; PCT application Ser. No. PCT/US2022/018563, filed on Mar. 2, 2022 and entitled “Methods and Compositions Used to Modify Chromatin Architecture to Regulate Phenotype in Aging and Cancer;” U.S. publication no. U.S. 2021/0180013 A1, filed on Apr. 23, 2019 and titled “Improved Methods for Inducing Tissue Regeneration and Senolysis in Mammalian Cells; U.S. published application no. 2020/0306296A1, filed on Mar. 27, 2020 and titled “Induced Tissue Regeneration Using Extracellular Vesicles”; and U.S. provisional application No. 63/155,628, filed Mar. 2, 2021 and titled “Methods and Compositions Used to Modify Chromatin Architecture to Regulate Phenotype in Aging and Cancer”; PCT application Ser. No. PCT/US2022/046737, filed on Oct. 14, 2022 and titled “Methods for the Temporal Regulation of Reprogramming Factors in Mammalian Cells”; U.S. provisional patent application No. 63/256,286, filed on Oct. 15, 2021 and titled “Methods for Modulating the Regenerative Phenotype in Mammalian Cells,” the contents of which is incorporated herein by reference). Genes commonly up-regulated in the embryonic/regenerative state include: AC108142.1, ADGRV1, AFF3, AGA, ALDHSA1, ALX1, AMH, AQP7P1, AQP7P3, 84GALNT4, BAHD1, BBOX1, C11orf35, C14orf39, CASC9, CBCAQH03 5, CBX2, CCDCl44NL, CHKB-CPT18, CHRM3, CPAMDB, CPT18, DLX1, DOC2GP, DPY19L2, DRD1/P, DSG2, F2RL2, FAM157A, FAM1578, FAR2P1, FAR2P3, FIRRE, FOXD1, FOXD4L4, FSIP2, GDF1, GRIN38, H2BFXP, /GF2BP1, L3MBTL1, LIN288, LINC00648, LINC00649, LINC01021, LINC02315, LMNB1, LOC644919, LOC728755, LOC791120, MED15P9, MN1, MN1, NAALAD2, Nb1a10527, OXTR, PAQR6, PCAT7, PCDHA10, PCDHA11, PCDHA2, PCDHA3, PCDHA4, PCDHAS, PCDHAC1, PCDHB10, PCDHB14, PCDHB16, PCDHB17, PCDHB2, PCDHB2, PCDHBS, PCDHB9, PCDHGB4, PCDHGB6, PKP3, PLPPR3, POTEE, POTEF, POU2F1, PRRSL, RAB3/P, RGPD1, RGPD2, 5/X1, SLCO1A2, TCF3, TSPAN11, TUBB28, WDR72, WRN, WSB1, ZCCHC18, ZNF497, and ZNF853.

Genes commonly up-regulated in the adult/nonregenerative state include: ACAT2, ADIRF, ALS2CR11, ANKRD65, ANKRD7, BACE2, BHMT2, C10orf1 1, C18orf56, C22orf26, CADPS2, CALHM2, CAT, CCDCl25, CCDCl44B, CCDCl44B, CCDC36, CCDC89, CLDNJJ, COMT, COX7A1, CTSF, DDX43, DNAJC15, DYNLT3, EGFLAM, ELOVL6, ESPNL, FAM24B, FDPS, FGF7, FKBP9L, FLG-ASI, FRG1B, GPAT2, GYPE, HENMT1, HIST2H2BA, IAH1, INSIG1, IRAK4, KRBOX1, LINC00654, LINC00839, LINC00865, LINC01116, IMNA, LOC100233156, LOC205251, LOC283788, LRRK2, AMOA, MAP10, MEG3, MEG8, MEG9, MIR4458HG, MIRLET7HG, NAALADL1, NKAPL, PAX8-AS1, PCDHGA12, PCDHGA2, PCDHGA6, PCDHGA7, PCDHGA9, PCDHGB3, PCDHGB5, PLPP7 (PAPPDC3), POMC, PRPH2, PRR34, PRR34-AS1, PRSS3, PSMD5, PTCHD3, PTCHD3PI, RPJJ-134021.1, RP5-1043Ll3.1, RPS7, SHMTJ, SPESP1, SVIL-ASI, TEKT4P2, TRJM4, TSPYL5, USP32P1, ZNF280D, ZNF300P 1, ZNF572, ZNF578, ZNF585B, ZNF736, and ZNF790-AS1.

Uses of iTR-Modified Cells, Tissues, and Organs of Postmortem Animals

### Pharmaceutical Compositions and Methods

Compositions described herein include populations of cells, tissues, and organs of postmortem animals modified by iTR and compositions for the application of iTR to the body. Said populations of cells, tissues, and organs modified by iTR have a variety of different uses. Non-limiting examples of such uses are discussed herein. In some embodiments, iTR is used to enhance regeneration of an organ or tissue thereby allowing the animal to be resuscitated. In some embodiments, iTR is applied in postmortem animals to populations of cells, tissues, and organs within a postmortem metazoan animal including humans to enhance regeneration of a limb, digit, cartilage, heart, blood vessel, bone, esophagus, stomach, liver, gallbladder, pancreas, intestines, rectum, anus, endocrine gland (e.g., thyroid, parathyroid, adrenal, endocrine portion of pancreas), skin, hair follicle, thymus, spleen, skeletal muscle, focal damaged cardiac muscle, smooth muscle, brain, spinal cord, peripheral nerve, ovary, fallopian tube, uterus, vagina, mammary gland, testes, vas deferens, seminal vesicle, prostate, penis, pharynx, larynx, vocal cords, trachea, bronchi, lungs, kidney, ureter, bladder, urethra, eye (e.g., retina, cornea), or ear (e.g., organ of Corti).

In some embodiments, said iTR-modified cells, tissues, and organs are used in transplant therapy to improve function compared to normal popuilations of cells, tissues, and organs. In some embodiments, iTR is applied to cells, tissues, and organs of postmortem animals to enhance regeneration of a stromal layer, e.g., a connective tissue supporting the parenchyma of a tissue. In some embodiments, iTR cells, tissues, and organs of postmortem animals is used to enhance regeneration following surgery, e.g., surgery that entails removal of at least a portion of a diseased or damaged tissue, organ, or other structure following resection or decellularization of a tumor. For example, such surgery might remove at least a portion of a liver, lung, kidney, stomach, pancreas, intestine, mammary gland, ovary, testis, bone, limb, digit, muscle, skin, etc. In some embodiments, the surgery is to remove a tumor. In some embodiments, iTR-modified cells, tissues, and organs of postmortem animals are used to promote scarless regeneration of skin following trauma, surgery, disease, and burns in a postmortem body.

Enhancing regeneration can include any one or more of the following, in various embodiments: (a) increasing the rate ofregeneration; (b) increasing the extent ofregeneration; (c) promoting establishment of appropriate structure (e.g., shape, pattern, tissue architecture, tissue polarity) in a regenerating tissue or organ or other body structure; (d) promoting growth of new tissue in a manner that retains and/or restores function; e) expansion of the cells, tissues, and organs of postmortem animals:” to obtain more tissue for transplantation. While use of iTR-modified cells, tissues, and organs of postmortem animals to enhance regeneration is of particular interest, the invention encompasses use of iTR-modified cells, tissues, and organs of postmortem animals to enhance repair, closure of a wound, or wound healing in general, without necessarily producing a detectable enhancement of epimorphic regeneration as well as for reversing the aging of said cells, tissues, and organs. Thus, the invention provides methods of enhancing regeneration, age-reversal, repair or wound healing, wherein iTR is applied to cells, tissues, and organs of postmortem animals and is administered to a subject in need thereof according to any of the methods described herein.

In some embodiments, the invention provides a method of enhancing regeneration in postmortem cells, tissues, or organs, the method comprising administering an effective amount of an iTR factor to cells, tissues, and organs of postmortem animals. to the subject.

In some embodiments, an effective amount of a compound (e.g., an iTR formulation for the uses described herein for cells, tissues, and organs of postmortem animals) is an amount that results in an increased rate or extent of regeneration of damaged tissue as compared with a reference value (e.g., a suitable control value). In some embodiments, the reference value is the expected (e.g., average or typical) rate or extent ofregeneration in the absence of the application of iTR-inducing factors to cells, tissues, and organs of postmortem animals. (optionally with administration of a placebo and vehicle controls). In some embodiments, an effective amount of iTR-inducing factors applied to cells, tissues, and organs of postmortem animals wherein said cells, tissues, or organs are subsequently transplanted to another organism is an amount that results in an improved structural and/or functional outcome as compared with the expected (e.g., average or typical) structural or functional outcome in the absence of the compound. In some embodiments, an effective amount of cells, tissues, and organs of postmortem animals engrafted following reprogramming by iTR factors is that which restores normal physiological function in a week or multiple weeks following transplantation. The extent or rate of regeneration can be assessed based on dimension(s) or volume of regenerated tissue, for example. Structural and/or functional outcome can be assessed based on, e.g., visual examination (optionally including use of microscopy or imaging techniques such as X-rays, CT scans, MRI scans, PET scans) and/or by evaluating the ability of the tissue, organ, or other body part to perform one or more physiological processes or task(s) normally performed by such tissue, organ, or body part. Typically, an improved structural outcome is one that more closely resembles normal structure (e.g., structure that existed prior to tissue damage or structure as it exists in a normal, healthy individual) as compared with the structural outcome that would be expected (e.g., average or typical outcome) in the absence of treatment with an iTR-inducing factor.

One of ordinary skill in the art can select an appropriate assay or test for function. In some embodiments, an increase in the rate or extent of regeneration as compared with a control value is statistically significant (e.g., with a p value of <0.05, or with a p value of <0.01) and/or clinically significant. In some embodiments, an improvement in structural and/or functional outcome as compared with a control value is statistically significant and/or clinically significant. “Clinically significant improvement” refers to an improvement that, within the sound judgement of a medical or surgical practitioner, confers a meaningful benefit on a subject (e.g., a benefit sufficient to make the treatment worthwhile).

In some embodiments, the iTR-modified cells, tissues, and organs of postmortem animals are used to enhance skin regeneration in an allogeneic animal such as a human, e.g., after a burn (thermal or chemical), scrape injury, or other situations involving skin loss, e.g., infections such as necrotizing fasciitis or purpura fulminans. In some embodiments, a burn is a second or third degree burn. In some embodiments a region of skin loss has an area of at least IO cm². In one aspect, iTR-modified cells, tissues, and organs of postmortem animals are used to enhance regeneration of grafted skin in the recipient. In one aspect, iTR-modified cells, tissues, and organs of postmortem animals reduce excessive and/or pathological wound contraction or scarring following transplantation. In another aspect, said iTR-modified skin is from the scalp or other hair-bearing skin to restore youthful cosmetic appearance.

In some embodiments, iTR-modified cells, tissues, and organs of postmortem animals are used to enhance bone regeneration, e.g., in a situation such as non-union fracture, implant fixation, periodontal or alveolar ridge augmentation, craniofacial surgery, or other conditions in which generation of new bone is considered appropriate. In some embodiments, an iTR factor is applied to a site where bone regeneration is desired. In some embodiments, an iTR factor is incorporated into or used in combination with a bone graft material. Bone graft materials include a variety of ceramic and proteinaceous materials. Bone graft materials include autologous bone (e.g., bone harvested from the iliac crest, fibula, ribs, etc.), allogeneic bone from cadavers, and xenogeneic bone.

Synthetic bone graft materials include a variety of ceramics such as calcium phosphates (e.g. hydroxyapatite and tricalcium phosphate), bioglass, and calcium sulphate, and proteinaceous materials such as demineralized bone matrix (DBM). DBM can be prepared by grinding cortical bone tissues (generally to 100-500 mih sieved particle size), then treating the ground tissues with hydrochloric acid (generally 0.5 to I N). In some embodiments, an iTR factor is administered to a subject together with one or more bone graft materials. The iTR factor may be combined with the bone graft material (in a composition comprising an iTR factor and a bone graft material) or administered separately, e.g., after placement of the graft. In some embodiments, the invention provides a bone paste comprising an iTR factor. Bone pastes are products that have a suitable consistency and composition such that they can be introduced into bone defects, such as voids, gaps, cavities, cracks etc., and used to patch or fill such defects, or applied to existing bony structures. Bone pastes typically have sufficient malleability to permit them to be manipulated and molded by the user into various shapes. The desired outcome of such treatments is that bone formation will occur to replace the paste, e.g., retaining the shape in which the paste was applied. The bone paste provides a supporting structure for new bone formation and may contain substance(s) that promote bone formation. Bone pastes often contain one or more components that impart a paste or putty-like consistency to the material, e.g., hyaluronic acid, chitosan, starch components such as amylopectin, in addition to one or more of the ceramic or proteinaceous bone graft materials (e.g., DBM, hydroxyapatite) mentioned above.

In some embodiments, an iTR factor enhances the formation and/or recruitment of osteoprogenitor cells capable of morphogenesis of the normal architecture of joints, tendons, and ligaments from mesenchymal cells and/or enhances the differentiation of osteoprogenitor cells into cells that form new bone (osteoblasts) wherein said mesenchymal or osteoprogenitor cells reside and are reprogrammed by iTR factors in a postmortem animal such as a human. In some embodiments, an iTR factor is administered to a postmortem subject with osteopenia or osteoporosis, e.g., to enhance bone regeneration in the subject after resuscitation. In some embodiments an iTR factor is administered to a post-mortem patient with cancers of the bone (osteosarcoma).

In some embodiments, an iTR factor is used to enhance regeneration of a joint (e.g., a fibrous, cartilaginous, or synovial joint) in a postmortem animal or human. In some embodiments, the joint is an intervertebral disc. In some embodiments, a joint is a hip, knee, elbow, or shoulder joint. In some embodiments, iTR-modified cells, tissues, and organs of postmortem animals are used to enhance regeneration of dental and/or periodontal tissues or structures (e.g., pulp, periodontal ligament, teeth, periodontal bone). In some embodiments, iTR-modified cells, tissues, and organs of postmortem animals are used to reduce glial scarring in CNS and PNS injuries. In some embodiments, iTR-modified cells, tissues, and organs of postmortem animals are used to reduce adhesions and stricture formation in internal surgery associated with transplantation of said cells, tissues, and organs. In some embodiments, iTR-modified cells, tissues, and organs of postmortem animals are used to decrease scarring in transplanted tendon and ligament repair improving mobility. In some embodiments, iTR-modified cells and tissues from the eye are transplanted to reduce vision loss following eye injury or disease.

In some embodiments the postmortem animal including humans is in the postnatal stage of development. In some embodiments of the present invention, the postmortem animal including humans is in the prenatal stage of development (i.e. embryo or fetus).

The cells reprogrammed by iTR factors in postmortem animals including humans can comprise progenitor cells or stem cells, e.g., adult stem cells. As used herein, an fetal or adult stem cell is a cell that possesses at least the following properties: (i) self-renewal, i.e., the ability to go through numerous (although finite in the case of some animals such as humans) cycles of cell division while still maintaining an undifferentiated state; and (ii) multipotency or multidifferentiative potential, i.e., the ability to generate progeny of several distinct cell types (e.g., many, most, or all of the distinct cell types of a particular tissue or organ). An adult stem cell is a stem cell originating from non-embryonic tissues (e.g., fetal, post-natal, or adult tissues). As used herein, the term “progenitor cell” encompasses multipotent cells that are more differentiated than pluripotent stem cells but not fully differentiated. Such more differentiated cells (which may arise from embryonic progenitor cells) have reduced capacity for self-renewal as compared with embryonic progenitor cells.

In some embodiments, iTR-modified cells are mesenchymal progenitor cells, neural progenitor cells, endothelial progenitor cells, hair follicle progenitor cells, neural crest progenitor cells, mammary stem cells, lung progenitor cells (e.g., bronchioalveolar stem cells), muscle progenitor cells (e.g., satellite cells), adipose-derived progenitor cells, epithelial progenitor cells (e.g., keratinocyte stem cells), and/or hematopoietic progenitor cells (e.g., hematopoietic stem cells). In some embodiments, the progenitor cells comprise adult stem cells. In some embodiments, at least some of the cells are differentiated cells, e.g., chondrocytes, osteoblasts, keratinocytes, hepatocytes. In some embodiments, the cells comprise myoblasts. In some embodiments, the fetal or adult stem cells are reprogrammed by the iTR factors of the present invention into cells capable ofreverting to blastema cells that facilitate epimorphic regeneration in the tissue corresponding anatomically to the site from which they were derived in the postmortem animal.

In some embodiments the iTR-modified cells, tissues, and organs of postmortem animals are genetically modified to evade immune rejection and thereby facilitating their use as an allogeneic graft with minimized need for immunosuppresion. Said genetic modifications may include the modification or elimination of one or more HLA antigens or beta 2 microglobulin, the introduction of immune suppressive modulators such as PDI.PDLI, or the exogenous expression ofHLA-G.

In some embodiments, iTR-modified cells, tissues, and organs of postmortem animals are reprogrammed to a differentiated but immature (i.e. pre-EFT) phenotype capable of scarless regeneration. In some embodiments, iTR-modified cells, tissues, and organs of postmortem animals are reprogrammed to a pluripotent state capable of forming teratomas which are subsequently used as a source of cells, tissues, and organs for allogeneic transplantation. In some embodiments, iTR-modified cells, tissues, and organs of postmortem animals are administered in a composition (e.g., a solution) comprising one or more compounds that polymerizes or becomes cross-linked or undergoes a phase transition in situ following administration to a subject, typically forming a hydrogel. The composition may comprise monomers, polymers, initiating agents, cross-linking agents, etc. The composition may be applied (e.g., using a syringe) to an area where regeneration is needed, where it forms a gel in situ, from which an iTR factor is released over time. Gelation may be triggered, e.g., by contact with ions in body fluids or by change in temperature or pH, or by light, or by combining reactive precursors (e.g., using a multi-barreled syringe). (See, e.g., U.S. Pat. No. 6,129,761; Yu L, Ding J. Injectable hydrogels as unique biomedical materials. Chem Soc Rev. 37(8): 1473-81 (2008)). In some embodiments the hydrogel is a hyaluronic acid or hyaluronic acid and collagen I-containing hydrogel such as HyStem-C described herein. In some embodiments, the composition further comprises cells.

In some embodiments, iTR-modified cells, tissues, and organs of postmortem animals are modified to express transiently or constitutively the catalytic component of telomerase (TERT). Said modification may be achieved through the use of vectors as described herein introducing the TERT gene. The expression of TERT is especially useful during the extensive expansion of iTR-modified cells, tissues, and organs of postmortem animals, or when said cells, tissues, and organs of postmortem animals are obtained from an aged animal such as a human patient. The vector expressing TERT may be administered separately or at the same time the cells, tissues, and organs of postmortem animals are reprogrammed to a regenerative state.

Other inventive methods comprise the cryopreservation of iTR-modified cells, tissues, and organs of postmortem animals tissue for subsequent allo- or autologous transplantation. Said cryopreservation may include the use of vitrification. Other inventive methods comprise use of an iTR-modified cells, tissues, and organs of postmortem animals in the ex vivo production of living, functional tissues, organs, or cell-containing compositions to repair or replace a tissue or organ lost due to damage. For example, cells or tissues removed from an individual (either the future recipient, an individual of the same species, or an individual of a different species) may be cultured in vitro, optionally with an matrix, scaffold (e.g., a three dimensional scaffold) or mold (e.g., comprising a biocompatible, optionally biodegradable, material, e.g., a polymer such as HyStem-C), and their development into a regenerative and expandable tissue or organ can be promoted by contacting an iTR factor. The scaffold, matrix, or mold may be composed at least in part of naturally occurring proteins such as collagen, hyaluronic acid, or alginate (or chemically modified derivatives of any of these), or synthetic polymers or copolymers of lactic acid, caprolactone, glycolic acid, etc., or self-assembling peptides, or decellularized matrices derived from tissues such as heart valves, intestinal mucosa, blood vessels, and trachea. In some embodiments, the scaffold comprises a hydrogel. The scaffold may, in certain embodiments, be coated or impregnated with an iTR factor, which may diffuse out from the scaffold over time. After production ex vivo, the tissue or organ is grafted into or onto a subject. For example, the tissue or organ can be implanted or, in the case of certain tissues such as skin, placed on a body surface. The tissue or organ may continue to develop in vivo. In some embodiments, the tissue or organ to be produced at least in part ex vivo is a bladder, blood vessel, bone, fascia, liver, muscle, skin patch, etc. Suitable scaffolds may, for example, mimic the extracellular matrix (ECM).

Optionally, an iTR factor is administered to the subject prior to, during, and/or following grafting of the ex vivo generated iTR-modified cells, tissues, and organs of postmortem animals. In some aspects, a biocompatible material is a material that is substantially non-toxic to cells in vitro at the concentration used or, in the case of a material that is administered to a living subject, is substantially nontoxic to the subject's cells in the quantities and at the location used and does not elicit or cause a significant deleterious or untoward effect on the subject, e.g., an immunological or inflammatory reaction, unacceptable scar tissue formation, etc. It will be understood that certain biocompatible materials may elicit such adverse reactions in a small percentage of subjects, typically less than about 5%, 1%, 0.5%, or 0.1%.

In some embodiments, a matrix or scaffold coated or impregnated with an iTR factor or combinations of factors including those capable of causing a global pattern of iTR gene expression is implanted, optionally in combination with cells, into a subject in need of regeneration. The matrix or scaffold may be in the shape of a tissue or organ whose regeneration is desired. The cells may be stem cells of one or more type(s) that gives rise to such tissue or organ and/or of type(s) found in such tissue or organ.

In some embodiments, an iTR factor or combination of factors is administered directly to or near a site of tissue damage in a postmortem metazoan animal including a postmortem human. “Directly to a site of tissue damage” or synonymously “targeting” encompasses injecting a compound or composition into a site of tissue damage or spreading, pouring, or otherwise directly contacting the site of tissue damage with the compound or composition.

In some embodiments, administration is considered “near a site of tissue damage” if administration occurs within up to about IO cm away from a visible or otherwise evident edge of a site of tissue damage or to a blood vessel (e.g., an artery) that is located at least in part within the damaged tissue or organ. Administration “near a site of tissue damage” is sometimes administration within a damaged organ, but at a location where damage is not evident. In some embodiments, following damage or loss of a tissue, organ, or other structure, an iTR factor is applied to the remaining portion of the tissue, organ, or other structure. In some embodiments, an iTR factor is applied to the end of a severed digit or limb) that remains attached to the body, to enhance regeneration of the portion that has been lost. In some embodiments, the severed portion is reattached surgically, and an iTR factor is applied to either or both faces of the wound. In some embodiments, an iTR factor is administered to enhance engraftment or healing or regeneration of a transplanted organ or portion thereof. In some embodiments, an iTR factor is used to enhance nerve regeneration.

For example, an iTR factor may be infused into a severed nerve, e.g., near the proximal and/or distal stump. In some embodiments, an iTR factor is placed within an artificial nerve conduit, a tube composed of biological or synthetic materials within which the nerve ends and intervening gap are enclosed. The factor or factors may be formulated in a matrix to facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

In some embodiments the iTR factor is anti-Mullerian hormone (AMH) which may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or in vivo at a concentrations ranging from 0.05-SmM valproic acid, preferably 1-100 ng/ml, preferably 10 ng/mL.

In some embodiments the iTR factor is GFER (Augmenter of Liver Regeneration (ALR)) in either the shorter secreted form or the longer form that localizes to the mitochondrial intermembrane space which is expressed in relatively higher levels in embryonic tissue and may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or cells in tissues in vivo at a concentration ranging from 2-200 ng/ml, preferably 20 ng/mL.

In some embodiments the iTR factor is valproic acid and may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or cells in tissue in vivo at a concentration ranging from 0.05-5 mM, preferably 0.5 mM.

In some embodiments the iTR factor is any combination of valproic acid at a concentration of 0.05-5 mM, preferably 0.5 mM, GFER protein (either the long or short form) at a concentration of 2-200 ng/mL, preferably 20 ng/ml and AMH protein at a concentration of 1-100 ng/ml, preferably 10 ng/ml. Said combination of the factors valproic acid, GFER, and AMH and may or may not be formulated for localization and slow release in carboxymethyl hyaluronate crosslinked by PEGDA with carboxymethyl-modified gelatin (HyStem-C) to induce iTR.

In some embodiments, the gene LIN28B normally expressed primarily in the embryonic phases of development is exogenously expressed in a postmortem metazoan animal including a postmortem human targeting blood cell types including CD34+ hematopoietic cells to promote their proliferation and engraftment into bone marrow in vivo comparable to the proliferative and engraftment capacity of their fetal liver-derived counterparts.

In some embodiments, an iTR factor or combinations of factors are used in a postmortem metazoan animal including a postmortem human to promote production of hair follicles and or growth of hair.

In some embodiments, an iTR factor or combination of factors are administered to tissues in a postmortem metazoan animal including a postmortem human wherein said tissue is afflicted with age-related degenerative changes to regenerate youthful function. Said age-related degenerative changes includes by way of nonlimiting example, age-related macular degeneration, coronary disease, osteoporosis, osteonecrosis, heart failure, emphysema, peripheral artery disease, vocal cord atrophy, hearing loss, Alzheimer's disease, Parkinson's disease, skin ulcers, and other age-related degenerative diseases. In some embodiments, said iTR factors are co administered with a vector expressing the catalytic component of telomerase to extend cell lifespan.

The methods and compositions for delivery of the segmental reprogramming factors of the present invention also include the delivery of cDNAs encoding LIN28A, LIN28B, IGF2BP1, IGF2BP2, IGF2BP3, HMGA2, and TERT, individually or in defined combinations, using viral and non-viral vectors as defined herein. The present disclosure also relates to gene therapy compositions wherein the aforementioned genes have modifications in their 3′ UTRs that reduce their sensitivity to repression by members of the LET-7 family of miRNAs as defined herein. By way of non-limiting example, particular emphasis is placed on adeno-associated viral (AAV) vectors for in vivo and in vitro delivery to reverse developmental aging and induce transient and controlled segmental reprogramming of differentiated cells toward a regenerative state without inducing full pluripotency.

In certain embodiments, one or more cDNAs encoding: LIN28A, LIN28B, IGF2BP1, IGF2BP2, IGF2BP3, HMGA2, and/or TERT or LIN28A, LIN28B, IGF2BP1, IGF2BP2, IGF2BP3, HMGA2, and/or TERT modified in their 3′ UTRs to reduce their sensitivity to repression by members of the LET-7 family of miRNAs are delivered transiently or semi-transiently to mature somatic cells to induce a regenerative phenotype characterized by: increased proliferative capacity; reversal of markers of aging such as aging-associated DMRs; or senescent gene expression such as the markedly decreased expression of PCDHGB4 and PEAR1, or increased expression of the cell cycle inhibitors CDKN2A, CDKN2B, or SASP markers such as MMP3 and PLAU; decreased expression of fetal or adult-upregulated expression of COX7A1; and increased telomerase activity within controlled bounds in cells treated with TERT vectors.

Segmental reprogramming, as used herein, refers to partial remodeling of transcriptional and post-transcriptional regulatory networks sufficient to enhance regenerative capacity without induction of pluripotency markers such as expression of OCT4, SOX2, or NANOG.

Vectors used to deliver the segmental reprogramming factors of the present invention may include, without limitation all methods that allow the transport of a nucleic acid such as a cDNA encoding LIN28A, LIN28B, IGF2BP1, IGF2BP2, IGF2BP3, HMGA2, and/or TERT or LIN28A, LIN28B, IGF2BP1, IGF2BP2, IGF2BP3, HMGA2, and/or TERT modified in their 3′ UTRs to reduce their sensitivity to repression by members of the LET-7 family of miRNAs. Said vectors include, without limitation, viral vectors or a virus or portion thereof (e.g., a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral) capsids. Plasmid vectors typically include an origin of replication and one or more selectable markers. Plasmids may include part or all of a viral genome (e.g., a viral promoter, enhancer, processing or packaging signals, etc.).

Viruses or portions thereof that can be used to introduce nucleic acid molecules into cells are referred to as viral vectors. Useful viral vectors include adenoviruses, adeno-associated viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. Vectors often include one or more appropriately positioned sites for restriction enzymes, to facilitate introduction into the vector of the nucleic acid to be expressed. An expression vector comprises sufficient cis-acting elements for expression; other elements required or helpful for expression can be supplied by the host cell or in vitro expression system.

Various techniques may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”).

Markers can be used for the identification and/or selection of cells that have taken up the vector and, typically, express the nucleic acid. Cells can be cultured in appropriate media to select such cells and, optionally, establish a stable cell line.

In certain preferred embodiments, recombinant AAV (rAAV) vectors are used. An rAAV vector comprises: 5′ Inverted Terminal Repeat (ITR), promoter, a transgene cassette, a polyadenylation signal, and a 3′ ITR. Commonly the viral rep and cap genes are supplied in trans during packaging. Different serotypes may be selected depending on tissue targeting and the modification made to the vector to reduce immunogenicity. For example, AAV1 is reported to be effective in muscle, while AAV2 is reported to have broad tropism, AAV6 is reported to be effective in muscle and lung, AAV8 is reported to be effective in liver, AAV9 is reported to be effective in cardiac and CNS applications, while AAVrh10 is also reported to be effective in CNS applications. Capsid engineering is desirable to modify tropism and reduce neutralizing antibody susceptibility. In some embodiments, self-complementary AAV vectors are employed to improve onset of expression. Due to packaging constraints (~2.4 kb for scAAV), individual factors or truncated variants may be delivered separately. For larger genes such as TERT (~3.5 kb coding sequence), dual AAV systems may be used with overlapping sequence recombination, trans-splicing vectors, and hybrid ITR-mediated concatemerization.

Lentiviral vectors may be used for stable genomic integration in vitro. Third-generation, self-inactivating (SIN) lentiviral systems are preferred to reduce insertional mutagenesis risk.

Packaging components include: gag/pol, rev, and VSV-G envelope. Vectors can be produced by transient transfection of HEK293T cells and purified by ultracentrifugation or chromatography. Replication-deficient adenoviral vectors may be used for high-level transient expression without integration.

Other vector systems may be utilized as well including without limitation: herpes simplex virus vectors (CNS applications) and Sendai virus (non-integrating RNA virus).

Doggy bone DNA (often abbreviated dbDNA™) may also be used. Doggy bone DNA refers to a minimal, linear, covalently closed DNA vector produced enzymatically in vitro that contains only the expression cassette (promoter-transgene-polyA) flanked by closed hairpin ends. It is designed to eliminate bacterial backbone sequences present in conventional plasmids while maintaining stability and transgene expression capacity. A typical doggy bone vector comprises: Hairpin end-Promoter-Coding sequence-Polyadenylation signal-Hairpin end. It is linear double-stranded DNA with no bacterial origin of replication, no antibiotic resistance genes, no plasmid backbone, covalently closed ends (no free DNA ends), and minimal CpG content (if designed accordingly). Because there are no free 5′ or 3′ DNA ends, the construct is resistant to exonuclease degradation compared to conventional linear DNA fragments. Doggy bone DNA is typically produced enzymatically without bacterial fermentation. A plasmid is designed containing: a promoter (e.g., CMV, CAG, EF1α, tissue-specific promoter to deliver to a particular tissue or a fetal or adult onset promoter such as the promoter of COX7A1 to drive expression only in non-regenerative cells further reducing risk of overly-reprogramming cells); a transgene (e.g., LIN28A, IGF2BP1, TERT, etc. as described herein); a polyA signal, protelomerase sequences, and flanking inverted repeat sequences for hairpin formation. The plasmid template can be amplified using: Phi29 DNA polymerase and isothermal rolling circle amplification. This generates long concatemers of the expression cassette. A site-specific protelomerase enzyme (e.g., TelN) cleaves at defined recognition sequences and simultaneously forms covalently closed hairpin ends. This converts concatemeric DNA into: Linear, closed-ended “doggy bone” DNA molecules. Purification steps may include: exonuclease digestion to remove template plasmid, anion exchange chromatography, tangential flow filtration, and endotoxin removal. The final product is a highly pure, minimal DNA vector suitable for in vivo delivery. Expression is typically transient but longer-lasting than naked linear DNA due to structural stability. Doggy bone DNA can be delivered via: lipid nanoparticles (LNPs) similar to mRNA LNP systems, ionizable lipid encapsulation, microfluidic mixing, electroporation for in vitro applications, polymeric nanoparticles, and biodegradable polymers such as collagen/hyaluronic hydrogels. Delivery routes include: intramuscular injection, intradermal injection, intraperitoneal injection, injection directly into the target tissue or artery supplying said tissue, CNS fluid, intravenous administration, and hydrodynamic tail vein injection (for animal preclinical applications).

Although AAV and doggy bone systems are emphasized herein, non-viral systems may also be used such as plasmid DNA with electroporation for in vitro applications; lipid nanoparticle (LNP)-encapsulated DNA; Polymeric nanoparticles; Minicircle DNA; sleeping Beauty transposon systems; and CRISPR-based targeted integration systems.

The seed target sequences of said 3′ UTR may be modified as described herein so as to decrease inhibition by LET-7 family members.

Regulatory elements may include promoter sequences such as CMV (strong, ubiquitous); CAG (ubiquitous hybrid); EF1α; tissue-specific promoters such as by way of nonliting examples: cardiac troponin T promoter for targeting expression in heart muscle cells, serum albumin promoter for expression in hepatocytes, and GFAP promoter for expression in CNS astrocytes. Enhancers may also be included to increase expression. Polyadenylation signals may include: SV40 polyA, BGH polyA. Expression may be accomplish with polycistronic expression, and multiple factors may be delivered via: 2A peptide sequences (e.g., P2A, T2A); internal ribosome entry sites (IRES); dual promoters; separate vectors co-administered such as co-administered AAV vectors, configurations such as: Promoter-LIN28A-P2A-IGF2BP1-P2A-HMGA2-P2A-H19-polyA. Promoters may be chosen so as to facilitate expression in adult non-regenerative cells by the use of promoters for genes not expressed in the embryonic regenerative state but expressed in fetal and adult states such as is the case with the COX7A1 promoter.

Furthermore, expression may be regulated by inducible systems in order to control duration and magnitude of expression such as: Tetracycline-inducible (Tet-On/Tet-Off) systems; Rapamycin-inducible dimerization systems; destabilization domains (e.g., FKBP-based); or miRNA-responsive regulatory elements.

Each cDNA encoding LIN28A, LIN28B, IGF2BP1-3, HMGA2, or TERT may be: codon optimized for human expression; depleted of cryptic splice sites; optimized for GC content; and engineered to remove destabilizing motifs. Additionally, post-transcriptional stabilization of LIN28A, LIN28B, IGF2BP1-3, HMGA2, or TERT mRNA may be employed by reduction of the levels of miRNAs targeting LIN28A, LIN28B, IGF2BP1-3, HMGA2 transcripts (described below); removal or mutation of LIN28A, LIN28B, IGF2BP1-3, HMGA2 3′UTR repressive elements (described below); m6A modification patterns that enhance LIN28A, LIN28B, IGF2BP1-3, HMGA2 mRNA stability (described below); RNA-binding proteins that protect LIN28A, LIN28B, IGF2BP1-3, HMGA2, or TERT transcripts; or post-translational stabilization of LIN28A, LIN28B, IGF2BP1-3, HMGA2, or TERT protein by: inhibition of ubiquitin ligases targeting the aforementioned proteins; proteasome inhibition; prevention of post-translational adducts that destabilize the protein(s). In addition, the gene FIRRE encodes a non-coding RNA that can stabilize IGF2BPs. It is expressed in hPSCs and expressed at much lower levels at or around the time of the EFT (FIG. 13). Therefore, the DNA or RNA from the gene FIRRE may be co-administered to improve segmental reprogramming to induce tissue regeneration and reverse developmental aging. Said DNA or RNA may furthermore be delivered as a circular molecule (e.g. circRNA).

Manufacture of vectors such as AAV vectors can be manufactured by triple transfection of HEK293 cells with: ITR-flanked transgene plasmid; Rep/Cap plasmid; helper plasmid; followed by incubation for 48-72 hours; followed by cell harvest and lysis.

Purification may be accomplished via: Iodixanol gradient ultracentrifugation; affinity chromatography (e.g., AVB Sepharose); and buffer exchange into physiological buffer.

Determination of viral genome (vg) titer can be assayed by qPCR. Quality control steps commonly would include endotoxin and sterility testing.

Lentivirus Production can be accomplished by co-transfection of packaging plasmids and transfer plasmid into HEK293T cells. Supernatant can be harvested at 48 and 72 hours. Concentration can be accomplished by ultracentrifugation with titer determination via p24 ELISA or functional transduction assay.

Dosing is typically with a multiplicity of infection (MOI) of 10³-10⁶ vg/cell in the case of AAV and 1-20 MOI with lentivirus vector. Incubation when applied intro is typically 48-120 hours with expression confirmation by: RT-qPCR, Western blot, or immunofluorescence.

The disclosed gene therapy systems, particularly AAV-based vectors, enable one skilled in the art to construct, produce, purify, and administer viral vectors encoding regenerative factors to achieve controlled, segmental reprogramming of mature cells in a postmortem mammal such as a human.

RNA Delivery Methods for Segmental Reprogramming to Induce Tissue Regeneration (iTR)

The present disclosure also relates to compositions and methods for stabilizing, formulating, and delivering RNA molecules encoding reprogramming and regenerative factors including LIN28A, LIN28B, IGF2BP1, IGF2BP2, IGF2BP3, HMGA2, H19, FIRRE, and TERT. The invention encompasses chemically modified RNAs, self-amplifying RNAs, circular RNAs, and genetically engineered variants thereof, formulated in lipid-based or extracellular vesicle-based delivery systems for in vitro and in vivo segmental reprogramming of mature cells to a regenerative state without full pluripotent conversion. In certain embodiments, combinations of RNAs encoding one or more of: LIN28A, LIN28B, IGF2BP1, IGF2BP2, IGF2BP3, HMGA2, and TERT are delivered transiently to induce partial epigenetic and post-transcriptional remodeling sufficient to reverse developmental aging to a pre-EFT phenotype or a pre-NT phenotype and thereby enhance regenerative capacity while avoiding full dedifferentiation to pluripotency. The segmental reprogramming induced by the administration of RNA will result in: increased proliferative competence, induction of a scarless regenerative state, and increased telomerase activity without permanent immortalization if and when TERT RNA is included.

In some embodiments, RNAs are capped, polyadenylated mRNAs encoding wild-type or modified proteins. Structural components may include a 5′ Cap (Cap 0, Cap 1, or Cap 2); an optimized 5′ UTR for translation efficiency; inclusion of a Kozak consensus sequence; codon-optimization of open reading frame; stabilized 3′ UTR; and a poly(A) tail (100-150 nt). To enhance stability and reduce innate immune activation, RNAs may incorporate chemically modified mRNA such as the substitution of N1-methylpseudouridine, pseudouridine, 5-methylcytidine, 2-thiouridine, and/or 5-methoxyuridine to reduce TLR3, TLR7, TLR8, and RIG-I activation and increase translational efficiency. In addition, modifications may be utilized in the ribose moiety such as the use of 2′-O-methyl, 2′-fluoro, or 2′-O-methoxyethyl (MOE) to increase resistance to nucleases. Furthermore, the diphosphate backbone may be chemically modified by incorporating phosphorothioate linkages (terminal or distributed) and partial backbone stabilization to reduce exonuclease degradation. In addition, the RNA can be formulated as circular RNA (circRNA)

Circular RNA (circRNA)

In some embodiments, RNAs encoded by LIN28A, LIN28B, IGF2BP1, IGF2BP2, IGF2BP3, HMGA2, H19, FIRRE, or TERT are generated as circular RNAs. Such circRNAs of the segmental reprogramming RNAs of the present invention may be manufactured using in vitro transcription from DNA template containing flanking self-splicing introns or ribozymes. Circularization is achieved via: a permuted intron-exon (PIE) system, treatment with T4 RNA ligase, removal of linear RNA via RNase R digestion followed by purification by chromatography. The CircRNAs of the present invention will demonstrate: increased half-life, reduced innate immune detection, and prolonged protein expression. Internal ribosome entry sites (IRES) or m6A-dependent translation motifs may be included. In addition, Self-Amplifying RNA (saRNA) may be utilized. In some embodiments, a replicon-based RNA derived from alphavirus backbones encodes the segmental reprogramming factor(s), enabling cytoplasmic amplification. Structural features include: nonstructural replicase genes, a subgenomic promoter, segmental reprogramming gene(s) ORF, and potentially modified nucleotides may be incorporated to reduce immunogenicity or reduced binding by LET-7 family members.

### Genetically Modified Variants

Variants for DNA or RNA-based delivery of the segmental reprogramming factors of the present invention may include: mutants of LIN28A, LIN28B, IGF2BP1, IGF2BP1, IGF2BP1, or HMGA2 with decreased LET-7 family binding affinity. In addition, destabilization domains (e.g., FKBP-derived) may be fused to permit pharmacologic control of protein half-life.

### Construction of LET-7 Family Member Resistant Genes and RNA

The segmental reprogramming factors of the present invention useful in performing iTR in vivo or in vitro preferably are stabilized by partially or entirely preventing the binding of LET-7 family member miRNAs (defined in Table I). The elimination or reduction of the ability of LET-7 family members (such as by way of nonlimiting example, hsa-let-7a-5p or hsa-miR-4458-5p) to repress LIN28A, LIN28B, IGF2BP1, IGF2BP2, IGF2BP3, or HMGA2 is accomplished by deletion of the seed targets for LET-7 miRNA or mir4458, or replacement of said seed target with an alternative sequence incapable of binding or with reduced binding ability to LET-7 miRNA family members. The common seed sequence of the LET-7 family members is GAGGUAG and the miRNA response element, also known as the target seed sequence is commonly CACCAUC. The seed targets that can be modified to reduced LET-7 family member binding are identified in Tables II-VII. By way of non-limiting example, in the case of the gene HMGA2, The 3′-UTR of the gene has seven seed targets for the hsa-let-7a-5p miRNA. These are at bases 21-28 (ctacctca), 1107-1113 (tacctca), 1256-1262 (ctacctc), 1619-1625 (tacctca), 1668-1674 (ctacctc), 2521-2527 (ctacctc), and 2541-47 (tacctca) of the 3′-UTR as shown in Table VII. These seed targets may be deleted in the construction of the gene sequence which is delivered by gene therapy, RNA therapy, of other gene delivery technologies described herein.

In one embodiment, the target seed sequences in one or more of IGF2BP1, IGF2BP2, IGF2BP3, LIN28A, LIN28B, or HMGA2 are modified by the deletion of 1,2,3,4,5,6 or 7 nucleotides and subsequently the modified genes or RNAs derived from said genes are used in segmental reprogramming as described above where the modification in the seed targets enhance their activity by reducing inhibition by LET-7 family members.

In another embodiment, the target seed sequences in one or more of IGF2BP1, IGF2BP2, IGF2BP3, LIN28A, LIN28B, or HMGA2 are modified by the substitution of 1,2,3,4,5,6 or 7 nucleotides unrelated to the LET-7 family seed target sequence or other miRNA seed target sequences, and subsequently the modified genes or RNAs derived from said genes are used in segmental reprogramming to enhance their activity.

In another embodiment, the target seed sequences in one or more of IGF2BP1, IGF2BP2, IGF2BP3, LIN28A, LIN28B, or HMGA2 are modified by the deletion or substitution of greater than seven nucleotides and subsequently the modified genes or RNAs derived from said genes are used in segmental reprogramming to enhance their activity.

In another embodiment, the target seed sequences in one or more of IGF2BP1, IGF2BP2, IGF2BP3, LIN28A, LIN28B, or HMGA2 are modified by the substitution of nucleotides recognized to promote m6A modification such as the sequence DRACH (D=A,G, or U); (R=A or G); A is the methylated adenosine (m6A); C=cytosine; and (H=A,C, or U). Said m6A modification will stabilize as oppose to inhibit the segmental reprogramming factor(s) described herein.

Members of the LET-7 family can also be inactivated using other methods, thereby effectively increasing the activity of the segmental reprogramming TR activators IGF2BP1, IGF2BP2, IGF2BP3, LIN28A, LIN28B, and HMGA2. In one such embodiment, antisense oligonucleotides (ASOs) can be designed that for dsRNA with pri- or pre-miRNAs of the LET-7 family. These “anti-miRs” bind a LET-7 family member's miRNA and promote its incorporation into RISC (RNA-induced silencing complex), effectively “silencing” its activity. These can include chemically modified oligonucleotides complementary to a segment of the mature LET-7 family member sequence. Locked nucleic acid (LNA) anti-LET-7 family member methods can be utilized and have the advantage of being highly stable and high-affinity binding. In another embodiment, 2′-O-methyl or 2′-O-methoxyethyl modifications can be used to improve nuclease resistance.

miRNA sponges or decoys may also be used to lower the levels of LET-7 family membes, thereby increasing the activity of IGF2BP1, IGF2BP2, IGF2BP3, LIN28A, LIN28B, or HMGA2. These may be synthetic transcripts containing multiple target seed sequences. These may include RNAs with concatemers of the target seed sequence. Said concatemers may include chemically modified oligonucleotides complementary to a segment of the mature LET-7 family member sequence. Locked nucleic acid (LNA) anti-LET-7 family member methods can be utilized and have the advantage of being highly stable and high-affinity binding. In another embodiment, 2′-O-methyl or 2′-O-methoxyethyl modifications can be used to improve nuclease resistance. In addition, said concatemers may be formulated as circRNA to further stabilize it. In addition, the concatemeric RNA may be expressed from plasmids or viral vectors.

In another embodiment, Tough Decoy (TuD) RNAs complementary to the LET-7 family member(s) may be utilized. This method has the advantage of forming with highly structured loops that can bind LET-7 family members very tightly.

A challenge to such LET-7 family inactivators is selectivity and the risk of off-taget effects.

Therefore design would include a match unique nucleotide differences outside the seed region.

In another embodiment competitive endogenous RNAs (ceRNAs) are utilized. One such gene is the maternally imprinted noncoding gene H19. This RNA effectively sponges LET-7 family members. Since it is normally expressed in fetal development and repressed in the neonate and subsequent adult (FIG. 12), it has a high probability of having an acceptable safety profile. The H19 gene can be delivered in the same manner as described above for DNA or RNA-based delivery of IGF2BP1, IGF2BP2, IGF2BP3, LIN28A, LIN28B, HMGA2, and/or TERT and used in combination with one or more of the nucleic acids. In this combination, preferably the combination would be LIN28B, IGF2BP1, H19, and TERT.

### Delivery Vehicles

The use of DNA and RNA as a means of delivering the segmental reprogramming factors of the present invention can be improved by the incorporation of said RNAs, including modified RNAs in lipid nanoparticles such as ionizable cationic lipid, DSPC or equivalent phospholipid, cholesterol, or PEG-lipid. Such lipid vesicles can be prepared by dissolving the lipids in ethanol; dissolving the RNA in acetate buffer (pH 4.0); mixing via microfluidic device at defined flow ratio; Dialyzing to physiological pH; and sterilizing by filtration (0.22 μm). The target particle size is tipically: 50-100 nm.

### Lipid Vesicle-Mediated Delivery

Liposomes may also be utilized to deliver the DNA and RNA segmental reprogramming factors of the present invention. Briefly, the liposomes are prepared using the thin-film hydration method: lipids are dissolved in chloroform; the resulting solution is evaporated to form a film; the film is hydrated with RNA or DNA solution; and the resulting solution is extruded through defined pore membranes.

### Exosome-Mediated Delivery

Exosomes may be used to transport RNA or DNA into cells. Said exosomes may be sourced from cultured cells of diverse types including fibroblasts, MSCs, hPS-derived embryonic progenitors, hPSCs, teratocarcinoma cell lines, and other cell types. Preferably said cells are immortal such as cancer cell lines, normal somatic cells transformed into immortal lines through the exogenous expression of TERT, or teratocarcinoma lines so as to allow them to be extensively genetically modified and used indefinitely for exosome production. Said cell lines used in the manufacture of exosomes may be engineered to over-express desired RNAs or miRNAs thereby loading eEVs or exosomes endogenously. Conditioned media containing said exosomes is collected and EVs partially purified by ultracentrifugation or size exclusion chromatography. RNAs including miRNAs may be loaded into preparations of EVs or exosomes by means known in the art such as by electroporation, sonication, or saponin permeabilization. Exosomes can be characterized by nanoparticle tracking analysis, Western blot for CD63, CD81, or RT-qPCR or RNA-seq for RNA cargo.

Furthermore, RNAs may be conjugated to GalNAc (liver targeting), cell-penetrating peptides, antibody fragments, or aptamers. Typical dosage concentration range is 10-500 ng RNA per 10⁵ cells or 0.1-5 μg/mL LNP formulation with incubation for 24-96 hours for in vitro applications.

In the case of in vivo applications, the formulation can be delivers intravenously, intramuscularly, intrathecally, intraperitoneally, as well as by other means. Dosage range is 0.05-3 mg/kg RNA with dosing daily, weekly or biweekly.

Furthermore, said EVs or exosomes may be selected that carry LET-7 family members for use in iTM and iCM as described below.

In some embodiments, iTR-modified cells, tissues, and organs of postmortem animals are administered to enhance replacement of cells that have been lost or damaged due to insults such as chemotherapy, radiation, or toxins. In some embodiments such cells are stromal cells of solid organs and tissues. Inventive methods of treatment can include a step of identifying or providing a subject suffering from or at risk of a disease or condition in which in which enhancing regeneration would be of benefit to the subject. In some embodiments, the subject has experienced injury (e.g., physical trauma) or damage to a tissue or organ. In some embodiments the damage is to a limb or digit. In some embodiments, a subject suffers from a disease affecting the cardiovascular, digestive, endocrine, musculoskeletal, gastrointestinal, hepatic, integumentary, nervous, respiratory, or urinary system. In some embodiments, tissue damage is to a tissue, organ, or structure such as cartilage, bone, heart, blood vessel, esophagus, stomach, liver, gallbladder, pancreas, intestines, rectum, anus, endocrine gland, skin, hair follicle, tooth, gum, lip, nose, mouth, thymus, spleen, skeletal muscle, smooth muscle, joint, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti). In some embodiments, iTR-modified cells, tissues, and organs of postmortem animals are administered to a subject at least once within approximately 2, 4, 8, 12, 24, 48, 72, or 96 hours after a subject has suffered tissue damage (e.g., an injury or an acute disease-related event such as a myocardial infarction or stroke) and, optionally, at least once thereafter. In some embodiments iTR-modified cells, tissues, and organs of postmortem animals are administered to a subject at least once within approximately 1-2 weeks, 2-6 weeks, or 6-12 weeks, after a subject has suffered tissue damage and, optionally, at least once thereafter.

In some embodiments of the invention, it may useful to stimulate or facilitate regeneration or de novo development of a missing or hypoplastic tissue, organ, or structure by, for example, removing the skin, removing at least some tissue at a site where regeneration or de novo development is desired, abrading a joint or bone surface where regeneration or de novo development is desired, and/or inflicting another type of wound on a subject. In the case ofregeneration after tissue damage, it may be desirable to remove (e.g., by surgical excision or debridement) at least some of the damaged tissue. In some embodiments, an iTR factor is administered at or near the site of such removal or abrasion.

In some embodiments, iTR-modified cells, tissues, and organs of postmortem animals are used to enhance generation of a tissue or organ in a subject in whom such tissue or organ is at least partially absent as a result of a congenital disorder, e.g., a genetic disease. Many congenital malformations result in hypoplasia or absence of a variety of tissues, organs, or body structures such as limbs or digits. In other instances a developmental disorder resulting in hypoplasia of a tissue, organ, or other body structure becomes evident after birth. In some embodiments, iTR-modified cells, tissues, and organs of postmortem animals are administered to a subject suffering from hypoplasia or absence of a tissue, organ, or other body structure, in order to stimulate growth or development of such tissue, organ, or other body structure. In some aspects, the invention provides a method of enhancing generation of a tissue, organ, or other body structure in a subject suffering from hypoplasia or congenital absence of such tissue, organ, or other body structure, the method comprising administering an iTR-modified cells, tissues, and organs of postmortem animals to the subject. In some embodiments, iTR-modified cells, tissues, and organs of postmortem animals are administered to the subject prior to birth, i.e., in utero. The various aspects and embodiments of the invention described herein with respect to regeneration are applicable to such de novo generation of a tissue, organ, or other body structure and are encompassed within the invention.

In some aspects, iTR-modified cells, tissues, and organs of postmortem animals are used to enhance generation of tissue in any of a variety of situations in which new tissue growth is useful at locations where such tissue did not previously exist. For example, generating bone tissue between joints is frequently useful in the context of fusion of spinal or other joints. iTR-modified cells, tissues, and organs of postmortem animals may be tested in a variety of animal models of regeneration. In one aspect, a iTR-modified cells, tissues, and organs of postmortem animals are tested in murine species. For example, mice can be wounded (e.g., by incision, amputation, transection, or removal of a tissue fragment). !TR-modified cells, tissues, and organs of postmortem animals are applied to the site of the wound and/or to a removed tissue fragment and its effect on regeneration is assessed.

The effect of a modulator of vertebrate TR can be tested in a variety of vertebrate models for tissue or organ regeneration. For example, fin regeneration can be assessed in zebrafish, e.g., as described in (Mathew L K, Unraveling tissue regeneration pathways using chemical genetics. J Biol Chem. 282 (48): 35202-10 (2007)), and can serve as a model for limb regeneration. Rodent, canine, equine, caprine, fish, amphibian, and other animal models useful for testing the effects of treatment on regeneration of tissues and organs such as heart, lung, limbs, skeletal muscle, bone, etc., are widely available. For example, various animal models for musculoskeletal regeneration are discussed in Tissue Eng Part B Rev. 16(1) (2010). A commonly used animal model for the study of liver regeneration involves surgical removal of a larger portion of the rodent liver. Other models for liver regeneration include acute or chronic liver injury or liver failure caused by toxins such as carbon tetrachloride. In some embodiments, a model for hair regeneration or healing of skin wounds involves excising a patch of skin, e.g., from a mouse. Regeneration of hair follicles, hair growth, re-epithelialization, gland formation, etc., can be assessed.

The compounds and compositions disclosed herein and/or identified using a method and/or assay system described herein may be administered by any suitable means such as orally, intranasally, subcutaneous!y, intramuscular!y, intravenous!y, intra-arterially, parenterally, intraperitoneally, intrathecally, intratracheally, ocularly, sublingually, vaginally, rectally, dermally, or by inhalation, e.g., as an aerosol. The particular mode selected will depend, of course, upon the particular compound selected, the particular condition being treated and the dosage required for therapeutic efficacy. The methods of this invention, generally speaking, may be practiced using any mode of administration that is medically or veterinarily acceptable, meaning any mode that produces acceptable levels of efficacy without causing clinically unacceptable (e.g., medically or veterinarily unacceptable) adverse effects.

Suitable preparations, e.g., substantially pure preparations, of one or more compound(s) may be combined with one or more pharmaceutically acceptable carriers or excipients, etc., to produce an appropriate pharmaceutical composition suitable for administration to a subject.

Such pharmaceutically acceptable compositions are an aspect of the invention. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, “Remington's Pharmaceutical Sciences”, E.W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types). Furthermore, compounds and compositions of the invention may be used in combination with any compound or composition used in the art for treatment of a particular disease or condition of interest.

In some embodiments, LIN28B is exogenously expressed in blood cell types including CD34+ hematopoietic cells to promote their proliferation and engraftment potential of postmortem bone marrow cells comparable to the proliferative and engraftment capacity of their fetal liver-derived counterparts.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's.

Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

For oral administration, compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP).

For administration by inhalation, inventive compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The present invention also contemplates delivery of compositions using a nasal spray or other forms of nasal administration. For topical applications, pharmaceutical compositions may be formulated in a suitable ointment, lotion, gel, or cream containing the active components suspended or dissolved in one or more pharmaceutically acceptable carriers suitable for use in such composition. For local delivery to the eye, the pharmaceutically acceptable compositions may be formulated as solutions or micronized suspensions in isotonic, pH adjusted sterile saline, e.g., for use in eye drops, or in an ointment, or for intra-ocularly administration, e.g., by injection. Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

In some embodiments, a composition includes one or more agents intended to protect the active agent(s) against rapid elimination from the body, such as a controlled release formulation, implants, microencapsulated delivery system, etc. Compositions may incorporate agents to improve stability (e.g., in the gastrointestinal tract or bloodstream) and/or to enhance absorption. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle, lipid, and/or polymer-based delivery systems are known in the art for delivery of siRNA. The invention contemplates use of such compositions. Liposomes or other lipid-based particles can also be used as pharmaceutically acceptable carriers.

Pharmaceutical compositions and compounds for use in such compositions may be manufactured under conditions that meet standards, criteria, or guidelines prescribed by a regulatory agency. For example, such compositions and compounds may be manufactured according to Good Manufacturing Practices (GMP) and/or subjected to quality control procedures appropriate for pharmaceutical agents to be administered to humans and can be provided with a label approved by a government regulatory agency responsible for regulating pharmaceutical, surgical, or other therapeutically useful products.

Pharmaceutical compositions of the invention, when administered to a subject for treatment purposes, are preferably administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects.

Different doses for human administration can be further tested in clinical trials in humans as known in the art. The dose used may be the maximum tolerated dose or a lower dose. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the particular disease or condition and its severity, the age, body weight, general health of the subject, etc.

It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. It will be understood that a therapeutic regimen may include administration of multiple doses, e.g., unit dosage forms, over a period of time, which can extend over days, weeks, months, or years. A subject may receive one or more doses a day, or may receive doses every other day or less frequently, within a treatment period. For example, administration may be biweekly, weekly, etc.

Administration may continue, for example, until appropriate structure and/or function of a tissue or organ has been at least partially restored and/or until continued administration of the compound does not appear to promote further regeneration or improvement. In some embodiments, a subject administers one or more doses of a composition of the invention to him or herself.

In some embodiments, two or more iTR-modified cells, tissues, and organs of postmortem animals or compositions are administered in combination, e.g., for purposes of enhancing regeneration.

Compounds or compositions administered in combination may be administered together in the same composition, or separately. In some embodiments, administration “in combination” means, with respect to administration of first and second compounds or compositions, administration performed such that (i) a dose of the second compound is administered before more than 90% of the most recently administered dose of the first agent has been metabolized to an inactive form or excreted from the body; or (ii) doses of the first and second compound are administered within 48, 72, 96, 120, or 168 hours of each other, or (iii) the agents are administered during overlapping time periods (e.g., by continuous or intermittent infusion); or (iv) any combination of the foregoing. In some embodiments, two or more iTR factors, or vectors expressing the catalytic component of telomerase and an iTR factor, are administered. In some embodiments iTR-modified cells, tissues, and organs of postmortem animals are administered in combination with a combination with one or more growth factors, growth factor receptor ligands (e.g., agonists), hormones (e.g., steroid or peptide hormones), or signaling molecules, useful to promote regeneration and polarity. Of particular utility are organizing center molecules useful in organizing regeneration competent cells such as those produced using the methods of the present invention. In some embodiments, a growth factor is an epidermal growth factor family member (e.g., EGF, a neuregulin), a fibroblast growth factor (e.g., any ofFGF1-FGF23), a hepatocyte growth factor (HGF), a nerve growth factor, a bone morphogenetic protein (e.g., any of BMP1-BMP7), a vascular endothelial growth factor (VEGF), a wnt ligand, a wnt antagonist, retinoic acid, NOTUM, follistatin, sonic hedgehog, or other organizing center factors. Those skilled in the art will recognize, or be able to ascertain using no more than routine experimentation, many equivalents to the specific embodiments of the invention described herein.

The scope of the present invention is not intended to be limited to the Description or the details set forth therein. Articles such as “a”, “an” and “the” may mean one or more than one unless indicated to the contrary or otherwise evident from the context. Certain of the inventive methods are often practiced using populations of cells, e.g., in vitro or in vivo.

Thus references to “a cell” should be understood as including embodiments in which the cell is a member of a population of cells, e.g., a population comprising or consisting of cells that are substantially genetically identical. However, the invention encompasses embodiments in which inventive methods is/are applied to an individual cell. Thus, references to “cells” should be understood as including embodiments applicable to individual cells within a population of cells and embodiments applicable to individual isolated cells.

Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. It is contemplated that all embodiments described herein are applicable to all different aspects of the invention. It is also contemplated that any of the embodiments can be freely combined with one or more other such embodiments whenever appropriate.

Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more elements or limitations found in any other claim that is dependent on the same base claim, and any claim that refers to an element present in a different claim can be modified to include one or more elements or limitations found in any other claim that is dependent on the same base claim as such claim. Furthermore, where the claims recite a composition, the invention provides methods of making the composition, e.g., according to methods disclosed herein, and methods of using the composition, e.g., for purposes disclosed herein. Where the claims recite a method, the invention provides compositions suitable for performing the method, and methods of making the composition. Also, where the claims recite a method of making a composition, the invention provides compositions made according to the inventive methods and methods of using the composition, unless otherwise indicated or unless one of ordinary skill in the art would recognize that a contradiction or inconsistency would arise. Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value). A “composition” as used herein, can include one or more than one component unless otherwise indicated. For example, a “composition comprising an activator or a TR activator” can consist or consist essentially of an activator of a TR activator or can contain one or more additional components. It should be understood that, unless otherwise indicated, an inhibitor or a TR inhibitor (or other compound referred to herein) in any embodiment of the invention may be used or administered in a composition that comprises one or more additional components including the presence of an activator of a TR activator.

### Example 1: Induced Tissue Regeneration Utilizing Klf4, Oct4, Sox2, Myc, and Tert in Postmortem Mammalian Cornea Followed by Transplantation

Mature Golden Syrian hamsters (Mesocricetus auratus) are utilized as an example of the reversal of developmental age and resulting induction of regeneration in a postmortem mammal maintained on life support by hypothermia and assisted respiration as described (Smith Audrey U. 1956. Studies on golden hamsters during cooling to and rewarming from body temperatures below 0° C.—I. Observations during chilling, freezing and supercooling Proc. R. Soc. Land B 145391-407 and Smith, Audrey U. Parkes, A. S. 1956. Studies on golden hamsters during cooling to and rewarming from body temperatures below 0° C.—II Observations during and after resuscitation Proc. R. Soc. Land B 145407-426) each incorporated by reference. Six hamsters (three female and three male) are utilized and the left eye serves as the treated group and the right eye serves as the control. Animals are anesthetized with a mixture of ketamine, xylazine and acepromazine and following surgery with analgesic as described (https://research.uoregon.edu/manage/integrity-compliance/animal-research/anesthesia-laboratory-animals) followed by decerebration as a model of brain death as described (Meehan, C., Mayr, K., Manuel, M. et al. Decerebrate mouse model for studies of the spinal cord circuits. Nat Protoc 12, 732-747 (2017). https://doi.org/10.1038/nprot.2017.001) incorporated by reference. The treated cohort is then treated simultaneously with two AAV9 constructs injected into the anterior chamber of the eye. The first vector KOSM is manufactured by inserting mouse Klf4, Oct4, Sox2 and Myc cDNA into an AAV9 expression plasmid. The second vector contains Tert cDNA inserted into an AAV9 expression plasmid. The third control vector is identical but without inserted cDNAs. All pAAVs, are packaged into serotype 2/2 AAV of titres: 5×10¹² genomes/ml. The expression vectors containing cDNAs and the control vectors are then injected into the anterior chamber of the eyes 5×10¹¹ gene copies (gc). The animals are then maintained at 0-2 deg C. for six days with assisted respiration, and corneas are surgically removed. Three of the recovered treated and control corneas are transplanted into recipient hamsters and removed in six months for comparative histology. RNA is extracted from the remaining three of the recovered treated and control corneas for mRNA sequencing to determine the expression of iTR gene markers as described herein.

### Example 2: Induced Tissue Regeneration Lin28a, Oct4, Nanog, Sox2, and Tert in Postmortem Mammalian Cornea Followed by Transplantation

Mature Golden Syrian hamsters (Mesocricetus auratus) are utilized as an example of the reversal of developmental age and resulting induction of regeneration in a postmortem mammal maintained on life support by hypothermia and assisted respiration as described (Smith Audrey U. 1956. Studies on golden hamsters during cooling to and rewarming from body temperatures below 0° C.—I. Observations during chilling, freezing and supercooling Proc. R. Soc. Land B 145391-407 and Smith, Audrey U. Parkes, A. S. 1956. Studies on golden hamsters during cooling to and rewarming from body temperatures below 0° C.—II Observations during and after resuscitation Proc. R. Soc. Land B 145407-426) each incorporated by reference. Six hamsters (three female and three male) are utilized and the left eye serves as the treated group and the right eye serves as the control.

Animals are anesthetized with a mixture of ketamine, xylazine and acepromazine and following surgery with analgesic as described (https://research.uoregon.edu/manage/integrity-compliance/animal-research/anesthesia-laboratory-animals) followed by decerebration as a model of brain death as described (Meehan, C., Mayr, K., Manuel, M. et al. Decerebrate mouse model for studies of the spinal cord circuits. Nat Protoc 12, 732-747 (2017). https://doi.org/10.1038/nprot.2017.001) incorporated by reference. The treated cohort is then treated simultaneously with two AAV9 constructs injected into the anterior chamber of the eye. The first vector KOSM is manufactured by inserting mouse Lin28a, Oct4, Nanog and Sox2 cDNA into an AAV9 expression plasmid. The second vector contains Tert cDNA inserted into an AAV9 expression plasmid. The third control vector is identical but without inserted cDNAs. All pAAVs, are packaged into serotype 2/2 AAV of titres: 5×10¹² genomes/ml. The expression vectors containing cDNAs and the control vectors are then injected into the anterior chamber of the eyes 5×10¹¹ gene copies (gc). The animals are then maintained at 0-2 deg C. for six days with assisted respiration, and corneas are surgically removed. Three of the recovered treated and control corneas are transplanted into recipient hamsters and removed in six months for comparative histology. RNA is extracted from the remaining three of the recovered treated and control corneas for mRNA sequencing to determine the expression of iTR gene markers as described herein.

What is claimed is:

1. A method of reprogramming one or more cell, tissue, or organ of a postmortem metazoan animal having cellular necrosis of the one or more cell, tissue or organ, to a regenerative state without inducing pluripotency of the one or more cell, tissue or organ, comprising the steps of:
a.) stabilizing the physiology of the metazoan animal using one or more life support technology;
b.) exposing the one or more cell, tissue or organ to one or more iTR factor gene, encoded iTR RNA and/or expressed iTR protein;
wherein the regenerative state is demonstrated by the ability of the one or more cell, tissue or organ to exhibit scarless regeneration;
wherein the iTR gene, iTR RNA and/or iTR protein is capable under other conditions of inducing pluripotency in the one or more cell, tissue or organ;
wherein the one or more iTR factor gene, encoded iTR RNA and/or expressed iTR protein is selected from the group of iTR genes consisting of OCT4, SOX2, KLF4, NANOG, ESRRB, NR5A2, CEBPA, MYC, LIN28A, TERT, and LIN28B, and their encoded iTR RNAs and iTR protein.

2. The method of claim 1, wherein the postmortem metazoan animal is human.

3. The method of claim 1, wherein the one or more iTR factor genes are delivered by viral gene therapy.

4. The method of claim 3, wherein the viral gene therapy vector is an adeno-associated virus.

5. The method of claim 1, wherein the one or more iTR factors are RNAs delivered by extracellular vesicles.

6. The method of claim 1, wherein the tissue of the postmortem animal 1s a cornea.

7. A method of expanding mammalian ex vivo cultured cells, tissues, and organs of postmortem animals. comprised of the steps of: (a) dissecting cells, tissues, and organs of postmortem animals previously reprogrammed for 6 days or more in conditions that promote tissue regeneration laterally from the epidermal surface to the base of the dermis, (b) culturing the tissue in vitro, (c) exposing the tissue to factors capable of reprogramming the cells to a state of induced tissue regeneration, (d) transplanting the cells, tissues, and organs of postmortem animals to a mammal to regenerate tissue.

8. The method of claim 7, wherein the mammal is human.

9. The method of claim 7, wherein the factors are delivered to the tissue by viral gene therapy.

10. The method of claim 9, wherein the viral gene therapy vector is an adeno-associated virus.

11. The method of claim 1, wherein the factors are RNAs delivered by extracellular vesicles.
